(19) United States
(12) Patent Application Publication
(10) Pub. No.: US 2026/0226023 A1
(43) Pub. Date: Aug. 6, 2026

(54) METHODS OF TREATING CARDIOMETABOLIC DISEASES

(71) Applicant: KayoThera Inc., Seattle, WA (US)

(72) Inventors: Mark ESPOSITO, Cazenovia, NY (US); John PIWINSKI, Lebanon, NJ (US)

(21) Appl. No.: 19/529,564

(22) Filed: Feb. 4, 2026

Related U.S. Application Data
(60) Provisional application No. 63/753,846, filed on Feb. 4, 2025.
(60) Provisional application No. 63/754,462, filed on Feb. 5, 2025.
(60) Provisional application No. 63/754,459, filed on Feb. 5, 2025.

(51) Int. Cl. C07D 401/12; A61K 31/4704; A61K 31/4709; A61P 3/10; A61P 9/04; C07D 215/38; C07D 401/14

(52) U.S. Cl. CPC C07D 401/12; A61K 31/4704; A61K 31/4709; A61P 3/10; A61P 9/04; C07D 215/38; C07D 401/14

(57) ABSTRACT

Provided herein are compounds of Formula (A), or a pharmaceutically acceptable salt, stereoisomer, tautomer, or deuterated form thereof; wherein: ring A, R^{A}, I¹, I², I³, Q, and Z are defined herein. Also provided herein are pharmaceutical compositions comprising a compound of Formula (A) or a pharmaceutically acceptable salt, stereoisomer, tautomer, or deuterated form thereof, and methods of using a compound of Formula (A) or a pharmaceutically acceptable salt, stereoisomer, tautomer, or deuterated form thereof e.g., in the treatment of cardiometabolic and cardiovascular diseases.

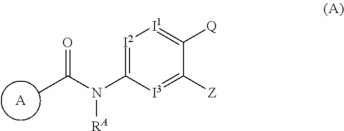

### CROSS-REFERENCES TO RELATED APPLICATIONS

The present application claims priority to U.S. Provisional Patent Application No. 63/753,846, filed Feb. 4, 2025, U.S. Provisional Patent Application No. 63/754,459, filed Feb. 5, 2025, and U.S. Provisional Patent Application No. 63/754,462, filed Feb. 5, 2025, both of which are hereby incorporated by reference.

### BACKGROUND

Aldehyde dehydrogenases (Aldhs, ALDHs) belong to a superfamily of NAD(P+)-dependent enzymes that play a role in the metabolism of aldehydes by irreversibly catalyzing the oxidation of both endogenously and exogenously produced aldehydes to their respective carboxylic acids. ALDHs have a broad spectrum of biological activities, including biosynthesis of retinoic acid (RA), oxidation of lipid peroxides, and alcohol metabolism, among others. ALDH, such as ALDH isoform 1a3 (ALDH1a3) is implicated in various diseases or disorders such as proliferative diseases or disorders, metabolic diseases or disorders, endothelial cell or smooth muscle cell diseases or disorders etc.

There is a need for compounds that selectively inhibit ALDH enzymes such ALDH1a3, as well as compositions and methods for treating a disease or disorder that is treatable by administration of an inhibit ALDH inhibitor such as an ALDH1a3 inhibitor, such as cardiometabolic and cardiovascular diseases.

### SUMMARY

The present disclosure is directed to, in part, compounds that inhibit one or more ALDH enzymes such as ALDH1a3, as well as pharmaceutical compositions thereof and uses thereof in treating various diseases or disorders. In particular embodiments, the compounds of the present disclosure are selective inhibitors of ALDH1a3. In one aspect, the use relates to methods of treating cardiometabolic syndrome, a diabetic kidney disease, a lipid disorder, muscle inflammation, sarcopenia or bone density loss, non-alcoholic fatty liver disease (NAFLD), non-alcoholic steatohepatitis (NASH), metabolic dysfunction-associated steatohepatitis (MASH), hypercholesterolemia, hypertriglyceridemia, familial cholesterolemia, familial triglyceridemia, or a cardiovascular disorder.

In embodiments, provided herein is a method of treating or preventing a disease or disorder in a subject in need thereof, the method comprising administering to the subject an effective amount of:
(i) a compound of Formula (A) or a pharmaceutically acceptable salt, stereoisomer, tautomer, or deuterated form thereof; or
(ii) a pharmaceutical composition comprising a compound of Formula (A) or a pharmaceutically acceptable salt, stereoisomer, tautomer, or deuterated form thereof, and a pharmaceutically acceptable carrier:

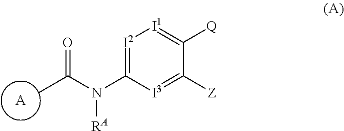

wherein:
ring A is an optionally substituted heteroaryl, an optionally substituted heterocyclyl or an optionally substituted aryl;
R^{A} is —H or —C₁₋₆alkyl;
I¹ is CR⁴ or N;
I² is CR³ or N;
I³ is CR⁷ or N;
R³ is —H, halo, —C₁₋₆ alkyl, or —C₁₋₆ haloalkyl;
R⁴ is —H, halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
Q and Z are joined to form a heterocyclyl and substituted with oxo; and wherein the heterocycle may be further optionally substituted;
R⁷ is —H, —C₁₋₆alkyl or halo;
R⁸ and R⁹ are each independently —H, —C₁₋₆ alkyl, aryl or heteroaryl; and
wherein the disease or disorder is cardiometabolic syndrome, a diabetic kidney disease, a lipid disorder, muscle inflammation, sarcopenia or bone density loss, non-alcoholic fatty liver disease (NAFLD), non-alcoholic steatohepatitis (NASH), metabolic dysfunction-associated steatohepatitis (MASH), hypercholesterolemia, hypertriglyceridemia, familial cholesterolemia, familial triglyceridemia, or a cardiovascular disorder.

In embodiments of the methods disclosed herein the compound of Formula (A) is a compound is of Formula (A-1):

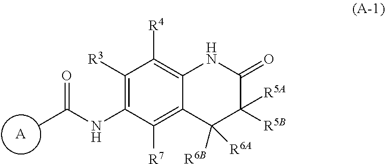

or a pharmaceutically acceptable salt, stereoisomer, tautomer, or deuterated form thereof,
wherein
ring A is heteroaryl or aryl, wherein the heteroaryl or aryl is optionally substituted with one or more substituents independently selected from the group consisting of halo, —NR⁸R⁹, —CN, —C₁₋₆ alkyl, —C₁₋₆ haloalkyl, —C₁₋₆ alkylene-carbocyclyl, or —C₁₋₆ alkylene-heterocyclyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R⁷ is —H, —C₁₋₆alkyl or halo;
R⁸ and R⁹ are each independently —H, —C₁₋₆ alkyl, aryl or heteroaryl; and
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring.

In embodiments, the compound of Formula (A-1) is a compound of Formula (III):

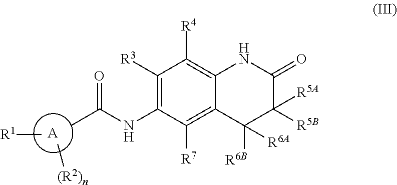

or a pharmaceutically acceptable salt, stereoisomer, tautomer, or deuterated form thereof
wherein: ring A, R¹, R², n, R³, R⁴, R^{5A}, R^{5B}, R^{6A}, R^{6B}, and R⁷ are defined herein.

In embodiments, the compound of Formula (I) is a compound of Formula (III-A):

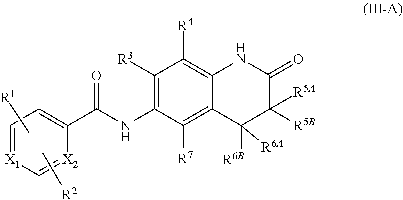

or a pharmaceutically acceptable salt, stereoisomer, tautomer, or deuterated form thereof;
wherein: R¹, R², R³, R⁴, R^{5A}, R^{5B}, R^{6A}, R^{6B}, R⁷, X¹ and X² are defined herein.

In embodiments, the compound of Formula (III) is a compound of Formula (III-B):

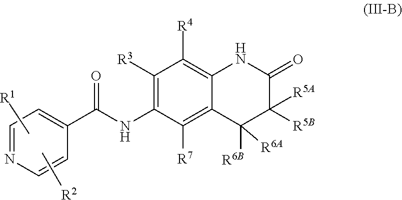

or a pharmaceutically acceptable salt, stereoisomer, tautomer, or deuterated form thereof;
wherein: R¹, R², R³, R⁴, R^{5A}, R^{5B}, R^{6A}, R^{6B}, and R⁷ are defined herein.

In embodiments, the compound of Formula (III) is a compound of Formula (III-C):

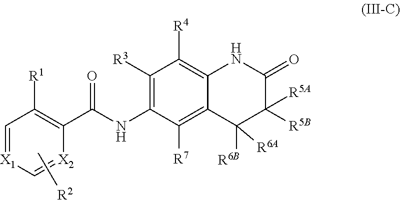

or a pharmaceutically acceptable salt, stereoisomer, tautomer, or deuterated form thereof;
wherein: R¹, R², R³, R⁴, R^{5A}, R^{5B}, R^{6A}, R^{6B}, R⁷, X¹ and X² are defined herein.

In embodiments, the compound of Formula (III) is a compound of Formula (III-D):

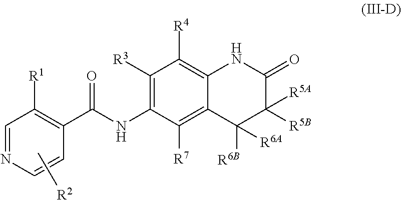

or a pharmaceutically acceptable salt, stereoisomer, tautomer, or deuterated form thereof;
wherein: R¹, R², R³, R⁴, R^{5A}, R^{5B}, R^{6A}, R^{6B}, and R⁷ are defined herein.

In embodiments, the compound of Formula (A-1) is a compound of Formula (IV):

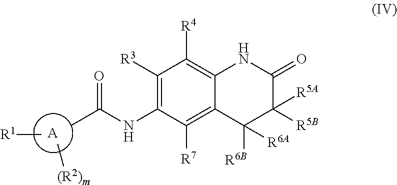

or a pharmaceutically acceptable salt, stereoisomer, tautomer, or deuterated form thereof;
wherein: ring A, R¹, R², m, R³, R⁴, R^{5A}, R^{5B}, R^{6A}, R^{6B}, and R⁷ are defined herein.

In embodiments, the compound of Formula (IV) is a compound of Formula (IV-A):

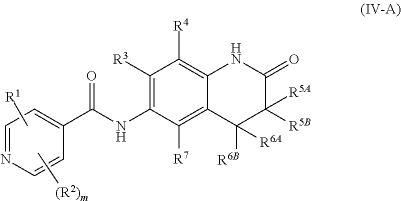

or a pharmaceutically acceptable salt, stereoisomer, tautomer, or deuterated form thereof;
wherein: R¹, R², m, R³, R⁴, R^{5A}, R^{5B}, R^{6A}, R^{6B}, and R⁷ are defined herein.

In embodiments, the compound of Formula (IV) is a compound of Formula (IV-B):

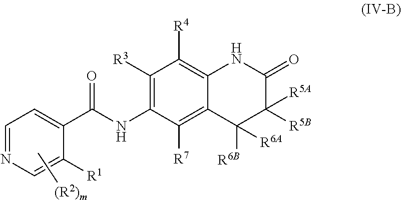

or a pharmaceutically acceptable salt, stereoisomer, tautomer, or deuterated form thereof;
wherein: R¹, R², m, R³, R⁴, R^{5A}, R^{5B}, R^{6A}, R^{6B}, and R⁷ are defined herein.

In embodiments, the lipid disorder is hyperlipidemia or dyslipidemia.

In embodiments, the cardiovascular disorder is pulmonary arterial hypertension, chronic heart failure, neointimal hyperplasia, Atherosclerotic Cardiovascular Disease (ASCVD), heart failure with preserved ejection fraction, ischemic heart disease or coronary artery disease.

### BRIEF DESCRIPTION OF THE DRAWINGS

FIG. 1A shows fasting blood glucose in ZDF rats treated with Compound 1 or vehicle control throughout the study disclosed in Example 19. All comparisons after day 0: p<0.001 by T-test.

FIG. 1B shows HbA1c (%) in ZDF rats treated with Compound 1 or vehicle control throughout the study disclosed in Example 19. All comparisons after day 0: p<0.001 by T-test.

FIG. 1C shows fasting plasma insulin in ZDF rats treated with Compound 1 or vehicle control throughout the study disclosed in Example 19. All comparisons after day 0: p<0.001 by T-test.

FIG. 2A shows water intake in ZDF rats treated with Compound 1 or vehicle control during the study disclosed in Example 19. Statistical significance at endpoints: p<0.001 by Student's T-test.

FIG. 2B shows body weight gain in ZDF rats treated with Compound 1 or vehicle control during the study disclosed in Example 19. Statistical significance at endpoints: p<0.001 by Student's T-test.

FIG. 3A shows liver mass normalized to total body mass and compared between Compound 1 treated and control groups (n=10) in the study disclosed in Example 19. Statistical significance at endpoints: p<0.001 by Student's T-test.

FIG. 3B shows kidney mass normalized to total body mass and compared between Compound 1 treated and control groups (n=10) in the study disclosed in Example 19. Statistical significance at endpoints: p<0.001 by Student's T-test.

FIG. 3C shows a molecular marker of liver injury (serum ALT) for ZDF rats treated with Compound 1 or vehicle control throughout the study described in Example 19. Statistical significance at endpoints: p<0.001 by Student's T-test.

FIG. 3D shows a molecular marker of kidney function (blood urea) for ZDF rats treated with Compound 1 or vehicle control throughout the study described in Example 19. Statistical significance at endpoints: p<0.001 by Student's T-test.

FIG. 4 creatine kinase (U/L) levels for ZDF rats treated with Compound 1 or vehicle control throughout the study described in Example 19 (−32%, p<0.05, at the last day of treatment).

FIG. 5A shows body weight for 16-week-old male severely diabetic db/db mice treated with Compound 1 (50 mg/kg) or vehicle control PO QD for 28 days in the study described in Example 20.

FIG. 5B shows glycated hemoglobin at experimental endpoint for severely diabetic db/db mice treated with Compound 1 (50 mg/kg) or vehicle control PO QD in the study described in Example 20. N=9 mice/group. p<0.001 by T-test.

FIG. 5C shows pancreatic islet area at experimental endpoint for severely diabetic db/db mice treated with Compound 1 (50 mg/kg) or vehicle control PO QD in the study described in Example 20. N=9 mice/group. p=0.05 by T-test.

FIG. 6A shows triglyceride levels in mice treated with Compound 1 or vehicle control PO QD in the study described in Example 20.

FIG. 6B shows LDL levels in mice treated with Compound 1 or vehicle control PO QD in the study described in Example 20.

FIG. 7A shows cardiac dynamics (end-systolic volume, ESV) for diabetic 7-week-old SDT fatty rats treated with vehicle control, Compound 1, and Semaglutide in the study described at Example 25.

FIG. 7B shows isovolumetric relaxation time (IVRT) for diabetic 7-week-old SDT fatty rats treated with vehicle control, Compound 1, and Semaglutide in the study described at Example 25.

FIG. 7C shows relative heart weight for diabetic 7-week-old SDT fatty rats treated with vehicle control, Compound 1, and Semaglutide in the study described at Example 25.

### DETAILED DESCRIPTION

### Definitions

For convenience, certain terms employed in the specification, examples and claims are collected here. Unless defined otherwise, all technical and scientific terms used in this disclosure have the same meanings as commonly understood by one of ordinary skill in the art.

The term “about” when immediately preceding a numerical value means a range of plus or minus an acceptable degree of variation in the art. In embodiments, the term “about” encompasses 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

“Halo” or “halogen” refers to fluorine (fluoro, —F), chlorine (chloro, —Cl), bromine (bromo, —Br), or iodine (iodo, —I).

As used herein, the term “alkyl” as used by itself or as part of another group refers to a straight- or branched-chain aliphatic saturated hydrocarbon. In embodiments, the alkyl is an alkyl containing one to twelve carbon atoms (i.e., C₁₋₁₂ alkyl) or the number of carbon atoms designated. In embodiments, the alkyl group is a straight chain C₁₋₁₀ alkyl group. In embodiments, the alkyl group is a branched chain C₁₋₁₀ alkyl group. In embodiments, the alkyl group is a straight chain C₁₋₆ alkyl group. In embodiments, the alkyl group is a branched chain C₁₋₆ alkyl group. In embodiments, the alkyl group is a straight chain C₁₋₄ alkyl group. In embodiments, the alkyl group is a branched chain C₁₋₄ alkyl group. For example, a C₁₋₄ alkyl group as used herein refers to a group selected from methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. In embodiments, “alkyl” is a straight-chain hydrocarbon. In some embodiments, “alkyl” is a branched hydrocarbon. Unless stated otherwise specifically in the specification, an alkyl group can be optionally substituted.

“Alkylene” or “alkylene chain” refers to a fully saturated, straight or branched divalent hydrocarbon chain radical. In embodiments, the alkylene has from one to twelve carbon atoms. Non-limiting examples of C₁-C₁₂ alkylene include methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

As used herein, the term “alkenyl” as used by itself or as part of another group refers to a straight- or branched-chain aliphatic hydrocarbon containing one or more, for example, one, two or three carbon-to-carbon double bonds. In embodiments, the alkenyl group is a C₂₋₆ alkenyl group. In embodiments, the alkenyl group is a C₂₋₄ alkenyl group. Non-limiting exemplary alkenyl groups include ethenyl, propenyl, isopropenyl, butenyl, sec-butenyl, pentenyl, and hexenyl. Unless stated otherwise specifically in the specification, an alkenyl group can be optionally substituted.

As used herein, the term “alkynyl” as used by itself or as part of another group refers to a straight- or branched-chain aliphatic hydrocarbon containing one or more, for example, one to three carbon-to-carbon triple bonds. In embodiments, the alkynyl has one carbon-carbon triple bond. In one embodiment, the alkynyl group is a C₂₋₆ alkynyl group. In embodiments, the alkynyl group is a C₂₋₄ alkynyl group. Non-limiting exemplary alkynyl groups include ethynyl, propynyl, butynyl, 2-butynyl, pentynyl, and hexynyl groups. Unless stated otherwise specifically in the specification, an alkynyl group can be optionally substituted.

As used herein, the term “alkoxy” as used by itself or as part of another group refers to a radical of the formula OR^{a1}, wherein R^{a1} is an alkyl, alkenyl, or alkynyl as defined herein. In embodiments, R^{a1} is an alkyl. Unless stated otherwise specifically in the specification, an alkoxy group can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon consisting solely of carbon and hydrogen atoms, which can include fused, spirocyclic, or bridged ring systems (e.g., fused, or bridged ring systems), having from three to twenty carbon atoms (e.g., 3-20, 3-10, 3-9 or 3-6), and which is attached to the rest of the molecule by a single bond. Monocyclic cycloalkyl include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms (e.g., 3-20, 3-10, 3-9 or 3-6), preferably having from three to ten carbon atoms, and which is attached to the rest of the molecule by a single bond. Monocyclic cycloalkenyl include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having from 3 to 20 carbon atoms and one or more carbon-carbon triple bonds, which can include fused or bridged ring systems, and which is attached to the rest of the molecule by a single bond. Monocyclic cycloalkynyls include, for example, cycloheptynyl, cyclooctynyl, and the like. Unless otherwise stated specifically in the specification, a cycloalkynyl group can be optionally substituted.

As used herein, the term “cycloalkoxy” as used by itself or as part of another group refers to a radical of the formula OR^{a1}, wherein R^{a1} is a cycloalkyl as defined herein. Unless stated otherwise specifically in the specification, a cycloalkoxy group can be optionally substituted.

As used herein, the term “haloalkyl” as used by itself or as part of another group refers to an alkyl substituted with one or more fluorine, chlorine, bromine and/or iodine atoms. In embodiments, the haloalkyl is an alkyl group substituted with one, two, or three fluorine atoms. In one embodiment, the haloalkyl group is a C₁₋₁₀ haloalkyl group. In embodiments, the haloalkyl group is a C₁₋₆ haloalkyl group. In embodiments, the haloalkyl group is a C₁₋₄ haloalkyl group. Unless otherwise stated specifically in the specification, a haloalkyl group can be optionally substituted.

“Heterocyclyl” or “heterocyclic” or “heterocycle” as used by itself or as part of another group refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In embodiments, each heteroatom is independently selected from nitrogen, oxygen, and sulfur. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is on the heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like. Unless otherwise stated specifically in the specification, a heterocyclyl can be optionally substituted.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In embodiments, an aryl group has six ring carbon atoms (“C₆ aryl”; e.g., phenyl). In embodiments, an aryl group has ten ring carbon atoms (“C₁₀ aryl”; e.g., naphthyl such as 1-naphthyl and 2-naphthyl). In embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise stated specifically in the specification, an aryl can be optionally substituted.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Unless otherwise stated specifically in the specification, a heteroaryl can be optionally substituted.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

An “optionally substituted” group as used herein means any of the above groups, such as an optionally substituted alkyl, optionally substituted alkylene, optionally substituted alkenyl, optionally substituted alkynyl, optionally substituted alkoxy, optionally substituted carbocyclyl, optionally substituted cycloalkyl, optionally substituted cycloalkenyl, optionally substituted cycloalkynyl, optionally substituted haloalkyl, optionally substituted heterocyclyl, optionally substituted aryl, optionally substituted aralkyl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable. Two of the optional substituents can join to form an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N(except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In embodiments, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, acyl, —C(O)-aryl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂), and —S(O)₂N(Y₁)(Y₂), wherein Y₁ and Y₂ can be the same or different and are independently selected from the group consisting of hydrogen, alkyl, aryl, cycloalkyl, and -alkylene-aryl.

Some examples of suitable substituents include, but not limited to, (C₁-C₈)alkyl groups, (C₂-C₈)alkenyl groups, (C₂-C₈)alkynyl groups, (C₃-C₁₀) cycloalkyl groups, halogen (F, Cl, Br or I), halogenated (C₁-C₈)alkyl groups (for example but not limited to —CF₃), —O—(C₁-C₈)alkyl groups, —OH, —S—(C₁-C₅)alkyl groups, —SH, —NH(C₁-C₅)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O)(C₁-C₈)alkyl groups, —NHC(O)(C₃-C₈) cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₅)alkyl)C(O)N((C₁-C₈)alkyl)₂ groups, —N((C₁-C₈)alkyl)C(O)NH((C₁-C₈)alkyl), —C(O)H, —C(O)(C₁-C₅)alkyl groups, —CN, —NO₂, —S(O)(C₁-C₅)alkyl groups, —S(O)₂ (C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂ (C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₆)alkyl groups, —C(═NOH)—(C₁-C₆)alkyl groups, or —C(═N—O—(C₁-C₆)alkyl)-(C₁-C₆)alkyl groups.

Exemplary carbon atom substituents include, but are not limited to, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl, or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3^{rd} edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trimethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkenyl, alkynyl, carbocyclic, cycloalkyl, alkoxy, cycloalkoxy, or heterocyclic group herein can be unsubstituted or substituted with 1, 2, 3, or 4 substituents independently selected from F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl (e.g., CF₃), C₁₋₄ alkoxy, and fluoro-substituted C₁₋₄ alkoxy.

In embodiments, the “optionally substituted” aryl or heteroaryl group herein can be unsubstituted or substituted with 1, 2, 3, or 4 substituents independently selected from F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from F, —OH, oxo (as applicable), C₁₋₄ alkyl, fluoro-substituted C₁₋₄ alkyl, C₁₋₄ alkoxy and fluoro-substituted C₁₋₄ alkoxy.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC) and the formation and crystallization of chiral salts; or isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “therapeutically effective” applied to dose or amount refers to that quantity of a compound or pharmaceutical formulation that is sufficient to result in a desired clinical benefit after administration to a patient in need thereof.

### Compounds

The compounds of the present disclosure are useful for inhibiting aldehyde dehydrogenase (e.g., ALDH1a3) and can be used for treating various diseases and disorders, such as cardiometabolic syndrome, a diabetic kidney disease, a lipid disorder, muscle inflammation, sarcopenia or bone density loss, non-alcoholic fatty liver disease (NAFLD), non-alcoholic steatohepatitis (NASH), metabolic dysfunction-associated steatohepatitis (MASH), hypercholesterolemia, hypertriglyceridemia, familial cholesterolemia, familial triglyceridemia, or a cardiovascular disorder.

### Compounds of Formula (A)

In embodiments, provided herein is a compound of Formula (A):

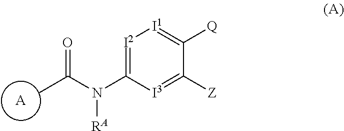

wherein:
ring A is an optionally substituted heteroaryl, an optionally substituted heterocyclyl or an optionally substituted aryl;
R^{A} is —H or —C₁₋₆alkyl;
I¹ is CR⁴ or N;
I² is CR³ or N;
I³ is CR⁷ or N;
R³ is —H, halo, —C₁₋₆ alkyl, or —C₁₋₆ haloalkyl;
R⁴ is —H, halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
Q and Z are joined to form a heterocyclyl and substituted with oxo; and wherein the heterocycle may be further optionally substituted;
R⁷ is —H, —C₁₋₆alkyl or halo; and
R⁸ and R⁹ are each independently —H, —C₁₋₆ alkyl, aryl or heteroaryl.

In embodiments, the compound of Formula (A) is a compound of Formula (A-1):

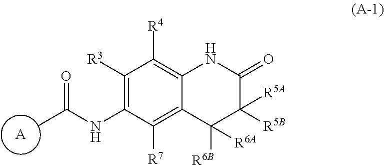

or a pharmaceutically acceptable salt, stereoisomer, tautomer, or deuterated form thereof,
wherein
ring A is heteroaryl or aryl, wherein the heteroaryl or aryl is optionally substituted with one or more substituents independently selected from the group consisting of halo, —NR⁸R⁹, —CN, —C₁₋₆ alkyl, —C₁₋₆ haloalkyl, —C₁₋₆ alkylene-carbocyclyl, or —C₁₋₆ alkylene-heterocyclyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —CN, —OH, —NH₂, —NH(alkyl), or —N(alkyl)₂; and
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring;
R⁷ is —H, —C₁₋₆alkyl or halo; and
R⁸ and R⁹ are each independently —H, —C₁₋₆ alkyl, aryl or heteroaryl.

### Compounds of Formula (I)

In embodiments, provided herein is a compound of Formula (I):

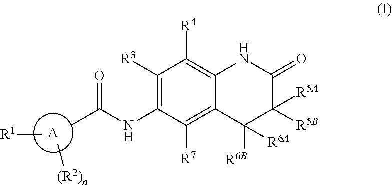

or a pharmaceutically acceptable salt, stereoisomer, tautomer, or deuterated form thereof;
wherein:
ring A is heteroaryl or aryl;
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
n is 1 or 2;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of the compounds of Formula (I), the compound is not:
(i) 5-Isothiazolecarboxamide, 3-methyl-N-(1,2,3,4-tetrahydro-8-methyl-2-oxo-6-quinolinyl)-4-(2,2,2-trifluoroethyl)-, having the structure:

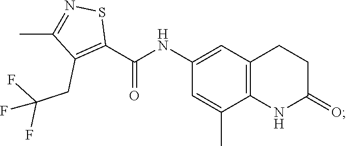 [SMILES: Cc1cc(NC(=O)c2snc(C)c2CC(F)(F)F)cc2c1NC(=O)CC2]

(ii) 5-Isothiazolecarboxamide, 3-cyano-N-(1,2,3,4-tetrahydro-8-methyl-2-oxo-6-quinolinyl)-4-(2,2,2-trifluoroethyl)-; having the structure:

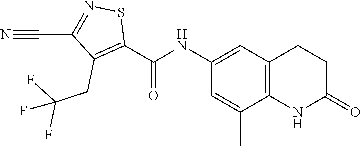 [SMILES: Cc1cc(NC(=O)c2snc(C#N)c2CC(F)(F)F)cc2c1NC(=O)CC2]

(iii) 4-Pyridinecarboxamide, 2-chloro-5-ethyl-N-(1,2,3,4-tetrahydro-8-methyl-2-oxo-6-quinolinyl)-; having the structure:

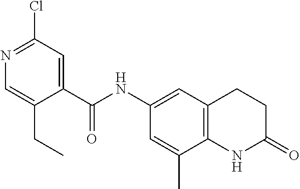 [SMILES: CCc1cnc(Cl)cc1C(=O)Nc1cc(C)c2c(c1)CCC(=O)N2]

(iv) 5H-Cyclopenta[b]pyridine-4-carboxamide, 6,6-difluoro-6,7-dihydro-N-(1,2,3,4-tetrahydro-8-methyl-2-oxo-6-quinolinyl)-; having the structure:

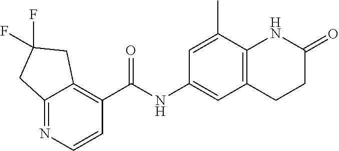 [SMILES: Cc1cc(NC(=O)c2ccnc3c2CC(F)(F)C3)cc2c1NC(=O)CC2]

or
4-Quinolinecarboxamide, 6,6-difluoro-5,6,7,8-tetrahydro-N-(1,2,3,4-tetrahydro-8-methyl-2-oxo-6-quinolinyl)-; having the structure:

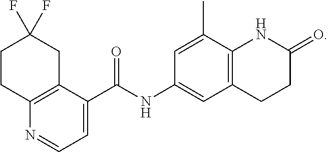 [SMILES: Cc1cc(NC(=O)c2ccnc3c2CC(F)(F)CC3)cc2c1NC(=O)CC2]

In embodiments of the compounds of Formula (I), ring A is heteroaryl.

In embodiments of the compounds of Formula (I), ring A is heteroaryl comprising at least one nitrogen atom.

In embodiments of the compounds of Formula (I), ring A is a monocyclic heteroaryl.

In embodiments of the compounds of Formula (I), ring A is a monocyclic heteroaryl comprising at least one nitrogen atom.

In embodiments of the compounds of Formula (I), ring A is a 5- or 6-membered heteroaryl.

In embodiments of the compounds of Formula (I), ring A is not an isothiazole.

In embodiments of the compounds of Formula (I), ring A is a heteroaryl having one or more heteroatoms independently selected from N and O. In embodiments, ring A is a monocyclic heteroaryl having one or more heteroatoms independently selected from N and O. In embodiments, ring A is a 5- or 6-membered heteroaryl having one or more heteroatoms independently selected from N and O. In embodiments, ring A is a 6-membered heteroaryl having one or more heteroatoms independently selected from N and O.

In embodiments of the compounds of Formula (I), ring A is a heteroaryl having one or two heteroatoms independently selected from N and O. In embodiments of the compounds of Formula (I), ring A is a monocyclic heteroaryl having one or two heteroatoms independently selected from N and O. In embodiments of the compounds of Formula (I), ring A is a 5- or 6-membered heteroaryl having one or two heteroatoms independently selected from N and O. In embodiments, ring A is a 6-membered heteroaryl having one or two heteroatoms independently selected from N and O.

In embodiments of the compounds of Formula (I), ring A is a heteroaryl having one or two N atoms. In embodiments of the compounds of Formula (I), ring A is a monocyclic heteroaryl having one or two N atoms. In embodiments of the compounds of Formula (I), ring A is a 5- or 6-membered heteroaryl having one or two N atoms. In embodiments of the compounds of Formula (I), ring A is a 6-membered heteroaryl having one or two N atoms.

In embodiments of the compounds of Formula (I), ring A is a heteroaryl having one N atom. In embodiments of the compounds of Formula (I), ring A is a monocyclic heteroaryl having one N atom. In embodiments of the compounds of Formula (I), ring A is a 5- or 6-membered heteroaryl having one N atom.

In embodiments of the compounds of Formula (I), ring A is a 6-membered heteroaryl.

In embodiments of the compounds of Formula (I), ring A is 6-membered heteroaryl comprising at least one nitrogen atom.

In embodiments of the compounds of Formula (I), ring A is 6-membered heteroaryl comprising one or two nitrogen atoms.

In embodiments of the compounds of Formula (I), ring A is pyridyl.

In embodiments of the compounds of Formula (I), ring A is aryl.

In embodiments of the compounds of Formula (I), ring A is a phenyl.

In embodiments of the compounds of Formula (I), n is 1 or 2.

In embodiments of the compounds of Formula (I), n is 1.

In embodiments of the compounds of Formula (I), n is 2.

In embodiments, provided herein is a compound of Formula (I-A):

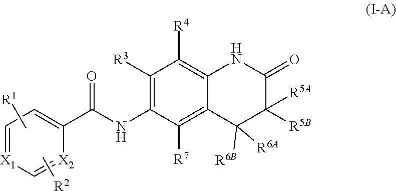

or a pharmaceutically acceptable salt, stereoisomer, tautomer, or deuterated form thereof;
wherein:
X¹ and X² are each independently CH or N;
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of Formula (I-A), X¹ is CH and X² is N.

In embodiments of Formula (I-A), X¹ is N and X² is CH.

In embodiments of Formula (I-A), X¹ is CH and X² is CH.

In embodiments of Formula (I-A), X¹ is N and X² is N.

In embodiments, provided herein is a compound of Formula (I-B):

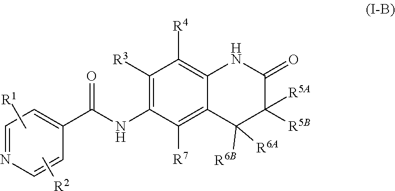

or a pharmaceutically acceptable salt, stereoisomer, tautomer, or deuterated form thereof wherein:
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of the compounds of Formula (I-A) or (I-B), the compound is not:
(i) 4-Pyridinecarboxamide, 2-chloro-5-ethyl-N-(1,2,3,4-tetrahydro-8-methyl-2-oxo-6-quinolinyl)-;
(ii) 5H-Cyclopenta[b]pyridine-4-carboxamide, 6,6-difluoro-6,7-dihydro-N-(1,2,3,4-tetrahydro-8-methyl-2-oxo-6-quinolinyl)-; or
(iii) 4-Quinolinecarboxamide, 6,6-difluoro-5,6,7,8-tetrahydro-N-(1,2,3,4-tetrahydro-8-methyl-2-oxo-6-quinolinyl)-.

In embodiments, provided herein is a compound of Formula (I-C):

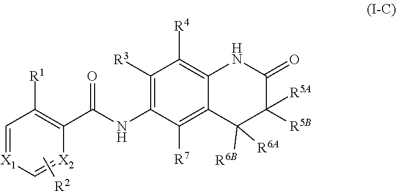

or a pharmaceutically acceptable salt, stereoisomer, tautomer, or deuterated form thereof;
wherein:
X¹ and X² are each independently CH or N;
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or
one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of Formula (I-C), X¹ is CH and X² is N.

In embodiments of Formula (I-C), X¹ is N and X² is CH.

In embodiments of Formula (I-C), X¹ is CH and X² is CH.

In embodiments of Formula (I-C), X¹ is N and X² is N.

In embodiments, provided herein is a compound of Formula (I-D):

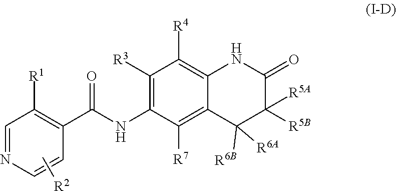

or a pharmaceutically acceptable salt, stereoisomer, tautomer, or deuterated form thereof
wherein:
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or
one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of the compounds of Formula (I), (I-A), (I-B), (I-C), or (I-D), R¹ is —C₁₋₆ haloalkyl, halo, or —NR⁸R⁹.

In embodiments of the compounds of Formula (I), (I-A), (I-B), (I-C), or (I-D), R¹ is —C₁₋₃ haloalkyl, halo, or —NR⁸R⁹.

In embodiments of the compounds of Formula (I), (I-A), (I-B), (I-C), or (I-D), R¹ is —C₁₋₆ haloalkyl.

In embodiments of the compounds of Formula (I), (I-A), (I-B), (I-C), or (I-D), R¹ is —C₁₋₃ haloalkyl.

In embodiments of the compounds of Formula (I), (I-A), (I-B), (I-C), or (I-D), R¹ is —CF₃, —CH₂CF₃, Cl, F, or —NH₂.

In embodiments of the compounds of Formula (I), (I-A), (I-B), (I-C), or (I-D), R¹ is —C₁₋₆ haloalkyl or —NR⁸R⁹.

In embodiments of the compounds of Formula (I), (I-A), (I-B), (I-C), or (I-D), R¹ is —C₁₋₃ haloalkyl or —NR⁸R⁹.

In embodiments of the compounds of Formula (I), (I-A), (I-B), (I-C), or (I-D), R¹ is —CF₃, —CH₂CF₃, or —NH₂.

In embodiments of the compounds of Formula (I), (I-A), (I-B), (I-C), or (I-D), R¹ is —CF₃ or —CH₂CF₃.

In embodiments of the compounds of Formula (I), (I-A), (I-B), (I-C), or (I-D), R¹ is —CF₃.

In embodiments of the compounds of Formula (I), (I-A), (I-B), (I-C), or (I-D), R¹ is —CH₂CF₃.

In embodiments of the compounds of Formula (I), (I-A), (I-B), (I-C), or (I-D), R² is independently halo, —CN, —C₁₋₆ alkyl, —C₁₋₆ alkylene-carbocyclyl, —C₁₋₆ alkylene-heterocyclyl, or —C₁₋₆ haloalkyl.

In embodiments of the compounds of Formula (I), (I-A), (I-B), (I-C), or (I-D), R² is independently —CN, —C₁₋₆ alkyl, —C₁₋₆ alkylene-carbocyclyl, —C₁₋₆ alkylene-heterocyclyl, or —C₁₋₆ haloalkyl.

In embodiments of the compounds of Formula (I), (I-A), (I-B), (I-C), or (I-D), R² is —CN or —C₁₋₆ alkyl.

In embodiments of the compounds of Formula (I), (I-A), (I-B), (I-C), or (I-D), R² is —CN or —C₁₋₃ alkyl.

In embodiments of the compounds of Formula (I), (I-A), (I-B), (I-C), or (I-D), R² is —CN, —CH₃, or —CH₂CH₃.

In embodiments of the compounds of Formula (I), (I-A), (I-B), (I-C), or (I-D), R² is —CN.

In embodiments of the compounds of Formula (I), (I-A), (I-B), (I-C), or (I-D), R² is —C₁₋₆ alkyl.

In embodiments of the compounds of Formula (I), (I-A), (I-B), (I-C), or (I-D), R² is —C₁₋₃ alkyl.

In embodiments of the compounds of Formula (I), (I-A), (I-B), (I-C), or (I-D), R² is —CH₃.

In embodiments of the compounds of Formula (I), (I-A), (I-B), (I-C), or (I-D), R² is —CH₂CH₃.

In embodiments of the compounds of Formula (I), (I-A), (I-B), (I-C), or (I-D), R¹ is —C₁₋₆ haloalkyl and R² is independently —CN or —C₁₋₆ alkyl.

In embodiments of the compounds of Formula (I), (I-A), (I-B), (I-C), or (I-D), R¹ is —C₁₋₆ haloalkyl and R² is —CN. In embodiments of the compounds of Formula (I), (I-A), (I-B), (I-C), or (I-D), R¹ is —C₁₋₃ haloalkyl and R² is —CN. In embodiments of the compounds of Formula (I), (I-A), (I-B), (I-C), or (I-D), R¹ is —CH₂CF₃, and R² is —CN.

In embodiments of the compounds of Formula (I), (I-A), (I-B), (I-C), or (I-D), R¹ is —C₁₋₆ haloalkyl and R² is —C₁₋₆ alkyl.

In embodiments of the compounds of Formula (I), (I-A), (I-B), (I-C), or (I-D), R³ is —H, halo, —C₁₋₆ alkyl, or —C₁₋₆ haloalkyl.

In embodiments of the compounds of Formula (I), (I-A), (I-B), (I-C), or (I-D), R³ is —H or halo.

In embodiments of the compounds of Formula (I), (I-A), (I-B), (I-C), or (I-D), R³ is —H.

In embodiments of the compounds of Formula (I), (I-A), (I-B), (I-C), or (I-D), R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂.

In embodiments of the compounds of Formula (I), (I-A), (I-B), (I-C), or (I-D), R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl.

In embodiments of the compounds of Formula (I), (I-A), (I-B), (I-C), or (I-D), R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —NR⁸C(O)C₁₋₆ alkyl, —NR⁸R⁹, —C(O)OH, —C(O)OC₁₋₆ alkyl, heterocyclyl, heteroaryl, or carbocyclyl.

In embodiments of the compounds of Formula (I), (I-A), (I-B), (I-C), or (I-D), R⁴ is halo, —CN, —C₁₋₆alkyl, —C₁₋₆ alkyl-CN, —C₁₋₆ haloalkyl, or carbocyclyl.

In embodiments of the compounds of Formula (I), (I-A), (I-B), (I-C), or (I-D), R⁴ is halo or —C₁₋₆alkyl.

In embodiments of the compounds of Formula (I), (I-A), (I-B), (I-C), or (I-D), R⁴ is —C₁₋₆alkyl.

In embodiments of the compounds of Formula (I), (I-A), (I-B), (I-C), or (I-D), R⁴ is —F or —CH₃.

In embodiments of the compounds of Formula (I), (I-A), (I-B), (I-C), or (I-D), R⁴ is —CH₃.

In embodiments of the compounds of Formula (I), (I-A), (I-B), (I-C), or (I-D), R³ is H, and R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂. In embodiments of the compounds of Formula (I), (I-A), (I-B), (I-C), or (I-D), R³ is H, and R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl. In embodiments of the compounds of Formula (I), (I-A), (I-B), (I-C), or (I-D), R³ is H, and R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —NR⁸C(O)C₁₋₆ alkyl, —NR⁸R⁹, —C(O)OH, —C(O)OC₁₋₆ alkyl, heterocyclyl, heteroaryl, or carbocyclyl. In embodiments of the compounds of Formula (I), (I-A), (I-B), (I-C), or (I-D), R³ is H, and R⁴ is halo, —CN, —C₁₋₆alkyl, —C₁₋₆ alkyl-CN, —C₁₋₆ haloalkyl, or carbocyclyl. In embodiments of the compounds of Formula (I), (I-A), (I-B), (I-C), or (I-D), R³ is H, and R⁴ is halo or —C₁₋₆alkyl. In embodiments of the compounds of Formula (I), (I-A), (I-B), (I-C), or (I-D), R³ is H, and R⁴ is —C₁₋₆alkyl. In embodiments of the compounds of Formula (I), (I-A), (I-B), (I-C), or (I-D), R³ is H, and R⁴ is —F or —CH₃. In embodiments of the compounds of Formula (I), (I-A), (I-B), (I-C), or (I-D), R³ is H, and R⁴ is —CH₃.

In embodiments of the compounds of Formula (I), (I-A), (I-B), (I-C), or (I-D), R¹ is —C₁₋₆ haloalkyl, R² is —CN, R³ is H, and R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl. In embodiments of the compounds of Formula (I), (I-A), (I-B), (I-C), or (I-D), R¹ is —C₁₋₆ haloalkyl, R² is —CN, R³ is H, and R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —NR⁸C(O)C₁₋₆ alkyl, —NR⁸R⁹, —C(O)OH, —C(O)OC₁₋₆ alkyl, heterocyclyl, heteroaryl, or carbocyclyl. In embodiments of the compounds of Formula (I), (I-A), (I-B), (I-C), or (I-D), R¹ is —C₁₋₆ haloalkyl, R² is —CN, R³ is H, and R⁴ is halo, —CN, —C₁₋₆alkyl, —C₁₋₆ alkyl-CN, —C₁₋₆ haloalkyl, or carbocyclyl. In embodiments of the compounds of Formula (I), (I-A), (I-B), (I-C), or (I-D), R¹ is —C₁₋₆ haloalkyl, R² is —CN, R³ is H, and R⁴ is halo or —C₁₋₆alkyl. In embodiments of the compounds of Formula (I), (I-A), (I-B), (I-C), or (I-D), R¹ is —C₁₋₆ haloalkyl, R² is —CN, R³ is H, and R⁴ is —C₁₋₆alkyl. In embodiments of the compounds of Formula (I), (I-A), (I-B), (I-C), or (I-D), R¹ is —C₁₋₆ haloalkyl, R² is —CN, R³ is H, and R⁴ is —F or —CH₃. In embodiments of the compounds of Formula (I), (I-A), (I-B), (I-C), or (I-D), R³ is H, and R⁴ is —CH₃. In embodiments of the compounds of Formula (I), (I-A), (I-B), (I-C), or (I-D), R¹ is —C₁₋₃ haloalkyl and R² is —CN. In embodiments of the compounds of Formula (I), (I-A), (I-B), (I-C), or (I-D), R¹ is —CH₂CF₃, and R² is —CN.

In embodiments of the compounds of Formula (I), (I-A), (I-B), (I-C), or (I-D), R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring.

In embodiments of the compounds of Formula (I), (I-A), (I-B), (I-C), or (I-D), R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 4-8 membered heterocyclic ring.

In embodiments of the compounds of Formula (I), (I-A), (I-B), (I-C), or (I-D), R^{5A}, R^{5B}, R^{6A}, and R^{6B} are each independently —H, halo, or —C₁₋₆ alkyl.

In embodiments of the compounds of Formula (I), (I-A), (I-B), (I-C), or (I-D), R^{5A}, R^{5B}, R^{6A}, and R^{6B} are—H.

In embodiments of the compounds of Formula (I), (I-A), (I-B), (I-C), or (I-D), R⁷ is —H, —C₁₋₆alkyl or halo.

In embodiments of the compounds of Formula (I), (I-A), (I-B), (I-C), or (I-D), R⁷ is —H or halo.

In embodiments of the compounds of Formula (I), (I-A), (I-B), (I-C), or (I-D), R⁷ is —H or—F.

In embodiments of the compounds of Formula (I), (I-A), (I-B), (I-C), or (I-D), R⁷ is —H or —C₁₋₆alkyl.

In embodiments of the compounds of Formula (I), (I-A), (I-B), (I-C), or (I-D), R⁷ is —H.

In embodiments of the compounds of Formula (I), (I-A), (I-B), (I-C), or (I-D), R⁸ and R⁹ are each independently —H, —C₁₋₆ alkyl, aryl or heteroaryl.

In embodiments of the compounds of Formula (I), (I-A), (I-B), (I-C), or (I-D), R⁸ and R⁹ are each independently —H or —C₁₋₆ alkyl.

In embodiments of the compounds of Formula (I), (I-A), (I-B), (I-C), or (I-D), R⁸ is —H or —C₁₋₆alkyl.

In embodiments of the compounds of Formula (I), (I-A), (I-B), (I-C), or (I-D), R⁸ is —H.

In embodiments of the compounds of Formula (I), (I-A), (I-B), (I-C), or (I-D), R⁸ is —C₁₋₆alkyl.

In embodiments of the compounds of Formula (I), (I-A), (I-B), (I-C), or (I-D), R⁹ is —H or —C₁₋₆alkyl.

In embodiments of the compounds of Formula (I), (I-A), (I-B), (I-C), or (I-D), R⁹ is —H.

In embodiments of the compounds of Formula (I), (I-A), (I-B), (I-C), or (I-D), R⁹ is —C₁₋₆alkyl.

In embodiments of the compounds of Formula (I), (I-A), (I-B), (I-C), or (I-D), R⁸ and R° are—H.

In embodiments, provided herein is a compound of Formula (I), (I-A), (I-B), (I-C), or (I-D), or a pharmaceutically acceptable salt or deuterated form thereof.

In embodiments, provided herein is a compound of Formula (I), (I-A), (I-B), (I-C), or (I-D), or a pharmaceutically acceptable salt thereof.

In embodiments, provided herein is a compound of Formula (I), (I-A), (I-B), (I-C), or (I-D).

### Compounds of Formula (II)

In embodiments, provided herein is a compound of Formula (II):

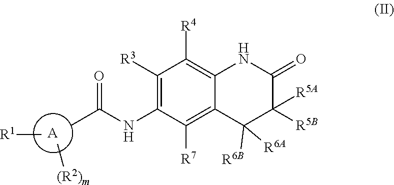

or a pharmaceutically acceptable salt, stereoisomer, tautomer, or deuterated form thereof;
wherein:
ring A is heteroaryl or aryl;
R¹ is —C₁₋₆ haloalkyl, halo or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
m is 0, 1 or 2;
R³ is —H, —C₁₋₆ alkyl, or —C₁₋₆ haloalkyl;
R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H or halo; and
R⁸ and R⁹ are each independently —H, —C₁₋₆alkyl, aryl, or heteroaryl.

In embodiments of the compounds of Formula (II), the compound is not:
(i) 5-Isothiazolecarboxamide, 3-methyl-N-(1,2,3,4-tetrahydro-8-methyl-2-oxo-6-quinolinyl)-4-(2,2,2-trifluoroethyl)-;
(ii) 5-Isothiazolecarboxamide, 3-cyano-N-(1,2,3,4-tetrahydro-8-methyl-2-oxo-6-quinolinyl)-4-(2,2,2-trifluoroethyl)-;
(iii) 4-Pyridinecarboxamide, 2-chloro-5-ethyl-N-(1,2,3,4-tetrahydro-8-methyl-2-oxo-6-quinolinyl)-;
(iv) 5H-Cyclopenta[b]pyridine-4-carboxamide, 6,6-difluoro-6,7-dihydro-N-(1,2,3,4-tetrahydro-8-methyl-2-oxo-6-quinolinyl)-; or
(v) 4-Quinolinecarboxamide, 6,6-difluoro-5,6,7,8-tetrahydro-N-(1,2,3,4-tetrahydro-8-methyl-2-oxo-6-quinolinyl)-.

In embodiments of the compounds of Formula (II), ring A is heteroaryl.

In embodiments of the compounds of Formula (II), ring A is heteroaryl comprising at least one nitrogen atom.

In embodiments of the compounds of Formula (II), ring A is a monocyclic heteroaryl.

In embodiments of the compounds of Formula (II), ring A is a monocyclic heteroaryl comprising at least one nitrogen atom.

In embodiments of the compounds of Formula (II), ring A is a 5- or 6-membered heteroaryl.

In embodiments of the compounds of Formula (II), ring A is a 6-membered heteroaryl.

In embodiments of the compounds of Formula (II), ring A is 6-membered heteroaryl comprising at least one nitrogen atom.

In embodiments of the compounds of Formula (II), ring A is 6-membered heteroaryl comprising one or two nitrogen atoms.

In embodiments of the compounds of Formula (II), ring A is not an isothiazole.

In embodiments of the compounds of Formula (II), ring A is a heteroaryl having one or more heteroatoms independently selected from N and O. In embodiments, ring A is a monocyclic heteroaryl having one or more heteroatoms independently selected from N and O. In embodiments, ring A is a 5- or 6-membered heteroaryl having one or more heteroatoms independently selected from N and O. In embodiments, ring A is a 6-membered heteroaryl having one or more heteroatoms independently selected from N and O.

In embodiments of the compounds of Formula (II), ring A is a heteroaryl having one or two heteroatoms independently selected from N and O. In embodiments, ring A is a monocyclic heteroaryl having one or two heteroatoms independently selected from N and O. In embodiments, ring A is a 5- or 6-membered heteroaryl having one or two heteroatoms independently selected from N and O. In embodiments, ring A is a 6-membered heteroaryl having one or two heteroatoms independently selected from N and O.

In embodiments of the compounds of Formula (II), ring A is a heteroaryl having one or two N atoms. In embodiments, ring A is a monocyclic heteroaryl having one or two N atoms. In embodiments, ring A is a 5- or 6-membered heteroaryl having one or two N atoms. In embodiments ring A is a 6-membered heteroaryl having one or two N atoms.

In embodiments of the compounds of Formula (II), ring A is pyridyl.

In embodiments of the compounds of Formula (II), ring A is aryl.

In embodiments of the compounds of Formula (II), ring A is a phenyl.

In embodiments, provided herein is a compound of Formula (II-A):

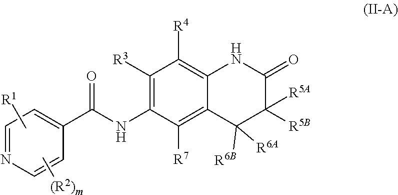

or a pharmaceutically acceptable salt, stereoisomer, tautomer, or deuterated form thereof; wherein:
R¹ is —C₁₋₆ haloalkyl, halo or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
m is 0, 1 or 2;
R³ is —H, —C₁₋₆ alkyl, or —C₁₋₆ haloalkyl;
R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H or halo; and
R⁸ and R⁹ are each independently —H, —C₁₋₆alkyl, aryl, or heteroaryl.

In embodiments of the compounds of Formula (II-A), the compound is not
(i) 4-Pyridinecarboxamide, 2-chloro-5-ethyl-N-(1,2,3,4-tetrahydro-8-methyl-2-oxo-6-quinolinyl)-;
(ii) 5H-Cyclopenta[b]pyridine-4-carboxamide, 6,6-difluoro-6,7-dihydro-N-(1,2,3,4-tetrahydro-8-methyl-2-oxo-6-quinolinyl)-; or
(iii) 4-Quinolinecarboxamide, 6,6-difluoro-5,6,7,8-tetrahydro-N-(1,2,3,4-tetrahydro-8-methyl-2-oxo-6-quinolinyl)-.

In embodiments, provided herein is a compound of Formula (II-B):

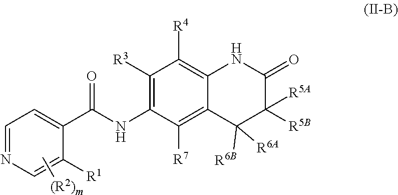

or a pharmaceutically acceptable salt, stereoisomer, tautomer, or deuterated form thereof; wherein:
R¹ is —C₁₋₆ haloalkyl, halo or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
m is 0, 1 or 2;
R³ is —H, —C₁₋₆ alkyl, or —C₁₋₆ haloalkyl;
R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H or halo; and
R⁸ and R⁹ are each independently —H, —C₁₋₆alkyl, aryl, or heteroaryl.

In embodiments of the compounds of Formula (II), (II-A), or (II-B), R¹ is —C₁₋₆ haloalkyl, halo or —NR⁸R⁹.

In embodiments of the compounds of Formula (II), (II-A), or (II-B), R¹ is —C₁₋₆ haloalkyl or —NR⁸R⁹.

In embodiments of the compounds of Formula (II), (II-A), or (II-B), R¹ is —C₁₋₃ haloalkyl or —NR⁸R⁹.

In embodiments of the compounds of Formula (II), (II-A), or (II-B), R¹ is —CF₃, —CH₂CF₃, or —NH₂.

In embodiments of the compounds of Formula (II), (II-A), or (II-B), R¹ is —C₁₋₆ haloalkyl.

In embodiments of the compounds of Formula (II), (II-A), or (II-B), R¹ is —C₁₋₃ haloalkyl.

In embodiments of the compounds of Formula (II), (II-A), or (II-B), R¹ is R¹ is-CH₂CF₃.

In embodiments of the compounds of Formula (II), (II-A), or (II-B), m is 0, 1 or 2.

In embodiments of the compounds of Formula (II), (II-A), or (II-B), m is 0.

In embodiments of the compounds of Formula (II), (II-A), or (II-B), m is 1.

In embodiments of the compounds of Formula (II), (II-A), or (II-B), m is 2.

In embodiments of the compounds of Formula (II), (II-A), or (II-B), R² is independently halo, —CN, —C₁₋₆ alkyl, —C₁₋₆ alkylene-carbocyclyl, —C₁₋₆ alkylene-heterocyclyl, or —C₁₋₆ haloalkyl.

In embodiments of the compounds of Formula (II), (II-A), or (II-B), R² is independently halo, —CN, —C₁₋₆ alkyl, or —C₁₋₆ haloalkyl.

In embodiments of the compounds of Formula (II), (II-A), or (II-B), R³ is —H, —C₁₋₆ alkyl, or —C₁₋₆ haloalkyl.

In embodiments of the compounds of Formula (II), (II-A), or (II-B), R³ is —H.

In embodiments of the compounds of Formula (II), (II-A), or (II-B), R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl.

In embodiments of the compounds of Formula (II), (II-A), or (II-B), R⁴ is —F, —CN, —OH, —C₁₋₆ alkyl-CN, —OC₁₋₆ alkyl, —C(O)NR⁸R⁹, —NR⁸C(O)C₁₋₆ alkyl, —NR⁸R⁹, —C(O)OH, —C(O)OC₁₋₆ alkyl, heterocyclyl, or heteroaryl.

In embodiments of the compounds of Formula (II), (II-A), or (II-B), R⁴ is —F, —CN, OC₁₋₆ alkyl, or —C(O)N(R⁸R⁹)₂.

In embodiments of the compounds of Formula (II), (II-A), or (II-B), R⁴ is a 5-6 membered heteroaryl. In embodiments, the heteroaryl is a 5-membered heteroaryl containing two nitrogen atoms.

In embodiments of the compounds of Formula (II), (II-A), or (II-B), R⁴ is —F, —CN, —OCH₃, —C(O)NH₂,

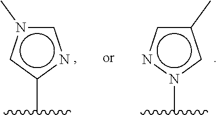

In embodiments of the compounds of Formula (II), (II-A), or (II-B), R⁴ is —F, —CN, —OCH₃, —C(O)NH₂,

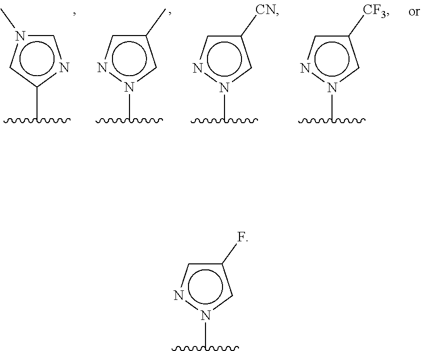

In embodiments of the compounds of Formula (II), (II-A), or (II-B), R^{5A}, R^{5B}, R^{6A}, and R^{6B} are each independently —H, halo, or —C₁₋₆ alkyl, or one of R^{5A} and R^{5B} is joined with one of R^{6A} and R6B to form an optionally substituted C₃₋₈ carbocyclic ring, or an optionally substituted 3-8 membered heterocyclic ring.

In embodiments of the compounds of Formula (II), (II-A), or (II-B), R^{5A}, R^{5B}, R^{6A}, and R^{6B} are each independently —H, halo, or —C₁₋₆ alkyl, or one of R^{5A} and R^{5B} is joined with one of R^{6A} and R^{6B} to form an optionally substituted C₃₋₈ carbocyclic ring, or an optionally substituted 4-8 membered heterocyclic ring.

In embodiments of the compounds of Formula (II), (II-A), or (II-B), R^{5A}, R^{5B}, R^{6A}, and R^{6B} are each independently —H, halo, or —C₁₋₆ alkyl.

In embodiments of the compounds of Formula (II), (II-A), or (II-B), R^{5A}, R^{5B}, R^{6A}, and R^{6B} are —H.

In embodiments of the compounds of Formula (II), (II-A), or (II-B), R⁷ is —H, —C₁₋₆alkyl or halo.

In embodiments of the compounds of Formula (II), (II-A), or (II-B), R⁷ is —H or halo.

In embodiments of the compounds of Formula (II), (II-A), or (II-B), R⁷ is —H or —F.

In embodiments of the compounds of Formula (II), (II-A), or (II-B), R⁷ is —H or —C₁₋₆alkyl.

In embodiments of the compounds of Formula (II), (II-A), or (II-B), R⁷ is —H.

In embodiments of the compounds of Formula (II), (II-A), or (II-B), R⁸ and R⁹ are each independently —H, —C₁₋₆ alkyl, aryl or heteroaryl.

In embodiments of the compounds of Formula (II), (II-A), or (II-B), R⁸ and R⁹ are each independently —H or —C₁₋₆ alkyl.

In embodiments of the compounds of Formula (II), (II-A), or (II-B), R⁸ is —H or —C₁₋₆alkyl.

In embodiments of the compounds of Formula (II), (II-A), or (II-B), R⁸ is —H.

In embodiments of the compounds of Formula (II), (II-A), or (II-B), R⁸ is —C₁₋₆alkyl.

In embodiments of the compounds of Formula (II), (II-A), or (II-B), R⁹ is —H or—C₁₋₆alkyl.

In embodiments of the compounds of Formula (II), (II-A), or (II-B), R⁹ is —H.

In embodiments of the compounds of Formula (II), (II-A), or (II-B), R⁹ is —C₁₋₆alkyl.

In embodiments of the compounds of Formula (II), (II-A), or (II-B), R⁸ and R⁹ are —H.

In embodiments, provided herein is a compound of Formula (II), (II-A), or (II-B), or a pharmaceutically acceptable salt or deuterated form thereof.

In embodiments, provided herein is a compound of Formula (II), (II-A), or (II-B), or a pharmaceutically acceptable salt thereof.

In embodiments, provided herein is a compound of Formula (II), (II-A), or (II-B).

### Compounds of Formula (III)

In embodiments, provided herein is a compound of Formula (III):

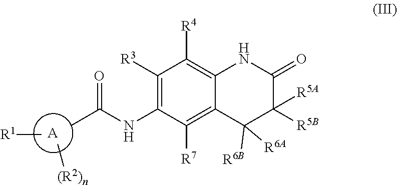

or a pharmaceutically acceptable salt, stereoisomer, tautomer, or deuterated form thereof; wherein:
ring A is heteroaryl or aryl;
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
n is 1 or 2;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of the compounds of Formula (III), the compound is not:
(i) 5-Isothiazolecarboxamide, 3-methyl-N-(1,2,3,4-tetrahydro-8-methyl-2-oxo-6-quinolinyl)-4-(2,2,2-trifluoroethyl)-, having the structure:

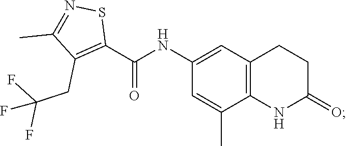 [SMILES: Cc1cc(NC(=O)c2snc(C)c2CC(F)(F)F)cc2c1NC(=O)CC2]

(ii) 5-Isothiazolecarboxamide, 3-cyano-N-(1,2,3,4-tetrahydro-8-methyl-2-oxo-6-quinolinyl)-4-(2,2,2-trifluoroethyl)-; having the structure:

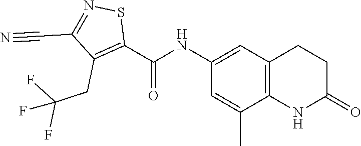 [SMILES: Cc1cc(NC(=O)c2snc(C#N)c2CC(F)(F)F)cc2c1NC(=O)CC2]

(iii) 4-Pyridinecarboxamide, 2-chloro-5-ethyl-N-(1,2,3,4-tetrahydro-8-methyl-2-oxo-6-quinolinyl)-; having the structure:

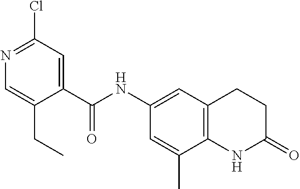 [SMILES: CCc1cnc(Cl)cc1C(=O)Nc1cc(C)c2c(c1)CCC(=O)N2]

(iv) 5H-Cyclopenta[b]pyridine-4-carboxamide, 6,6-difluoro-6,7-dihydro-N-(1,2,3,4-tetrahydro-8-methyl-2-oxo-6-quinolinyl)-; having the structure:

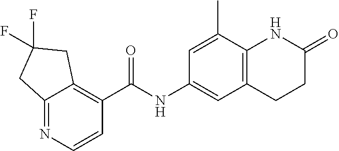 [SMILES: Cc1cc(NC(=O)c2ccnc3c2CC(F)(F)C3)cc2c1NC(=O)CC2]

or
4-Quinolinecarboxamide, 6,6-difluoro-5,6,7,8-tetrahydro-N-(1,2,3,4-tetrahydro-8-methyl-2-oxo-6-quinolinyl)-; having the structure:

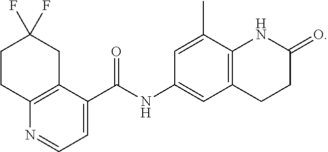 [SMILES: Cc1cc(NC(=O)c2ccnc3c2CC(F)(F)CC3)cc2c1NC(=O)CC2]

In embodiments of the compounds of Formula (III), ring A is heteroaryl.

In embodiments of the compounds of Formula (III), ring A is heteroaryl comprising at least one nitrogen atom.

In embodiments of the compounds of Formula (III), ring A is a monocyclic heteroaryl.

In embodiments of the compounds of Formula (III), ring A is a monocyclic heteroaryl comprising at least one nitrogen atom.

In embodiments of the compounds of Formula (III), ring A is a 5- or 6-membered heteroaryl.

In embodiments of the compounds of Formula (III), ring A is not an isothiazole.

In embodiments of the compounds of Formula (III), ring A is a heteroaryl having one or more heteroatoms independently selected from N and O. In embodiments, ring A is a monocyclic heteroaryl having one or more heteroatoms independently selected from N and O. In embodiments, ring A is a 5- or 6-membered heteroaryl having one or more heteroatoms independently selected from N and O. In embodiments, ring A is a 6-membered heteroaryl having one or more heteroatoms independently selected from N and O.

In embodiments of the compounds of Formula (III), ring A is a heteroaryl having one or two heteroatoms independently selected from N and O. In embodiments of the compounds of Formula (I-1), ring A is a monocyclic heteroaryl having one or two heteroatoms independently selected from N and O. In embodiments of the compounds of Formula (I-1), ring A is a 5- or 6-membered heteroaryl having one or two heteroatoms independently selected from N and O. In embodiments, ring A is a 6-membered heteroaryl having one or two heteroatoms independently selected from N and O.

In embodiments of the compounds of Formula (III), ring A is a heteroaryl having one or two N atoms. In embodiments of the compounds of Formula (III), ring A is a monocyclic heteroaryl having one or two N atoms. In embodiments of the compounds of Formula (III), ring A is a 5- or 6-membered heteroaryl having one or two N atoms. In embodiments of the compounds of Formula (III), ring A is a 6-membered heteroaryl having one or two N atoms.

In embodiments of the compounds of Formula (III), ring A is a heteroaryl having one N atom. In embodiments of the compounds of Formula (III), ring A is a monocyclic heteroaryl having one N atom. In embodiments of the compounds of Formula (III), ring A is a 5- or 6-membered heteroaryl having one N atom.

In embodiments of the compounds of Formula (III), ring A is a 6-membered heteroaryl.

In embodiments of the compounds of Formula (III), ring A is 6-membered heteroaryl comprising at least one nitrogen atom.

In embodiments of the compounds of Formula (III), ring A is 6-membered heteroaryl comprising one or two nitrogen atoms.

In embodiments of the compounds of Formula (III), ring A is pyridyl.

In embodiments of the compounds of Formula (III), ring A is aryl.

In embodiments of the compounds of Formula (III), ring A is a phenyl.

In embodiments of the compounds of Formula (III), n is 1 or 2.

In embodiments of the compounds of Formula (III), n is 1.

In embodiments of the compounds of Formula (III), n is 2.

In embodiments, provided herein is a compound of Formula (III-A):

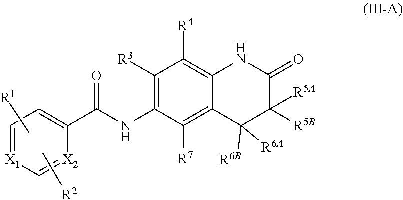

or a pharmaceutically acceptable salt, stereoisomer, tautomer, or deuterated form thereof;
wherein:
X¹ and X² are each independently CH or N;
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of Formula (III-A), X¹ is CH and X² is N.

In embodiments of Formula (III-A), X¹ is N and X² is CH.

In embodiments of Formula (III-A), X¹ is CH and X² is CH.

In embodiments of Formula (III-A), X¹ is N and X² is N.

In embodiments, provided herein is a compound of Formula (III-B):

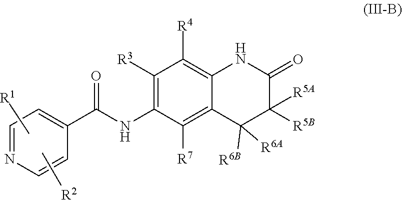

or a pharmaceutically acceptable salt, stereoisomer, tautomer, or deuterated form thereof
wherein:
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or
one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of the compounds of Formula (III-A) or (III-B), the compound is not:
(i) 4-Pyridinecarboxamide, 2-chloro-5-ethyl-N-(1,2,3,4-tetrahydro-8-methyl-2-oxo-6-quinolinyl)-;
(ii) 5H-Cyclopenta[b]pyridine-4-carboxamide, 6,6-difluoro-6,7-dihydro-N-(1,2,3,4-tetrahydro-8-methyl-2-oxo-6-quinolinyl)-; or
(iii) 4-Quinolinecarboxamide, 6,6-difluoro-5,6,7,8-tetrahydro-N-(1,2,3,4-tetrahydro-8-methyl-2-oxo-6-quinolinyl)-.

In embodiments, provided herein is a compound of Formula (III-C):

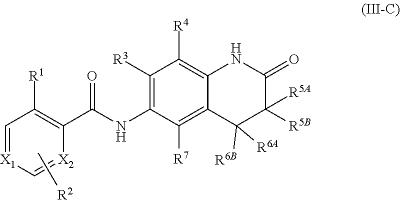

or a pharmaceutically acceptable salt, stereoisomer, tautomer, or deuterated form thereof;
wherein:
X¹ and X² are each independently CH or N;
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of Formula (III-C), X¹ is CH and X² is N.

In embodiments of Formula (III-C), X¹ is N and X² is CH.

In embodiments of Formula (III-C), X¹ is CH and X² is CH.

In embodiments of Formula (III-C), X¹ is N and X² is N.

In embodiments, provided herein is a compound of Formula (III-D):

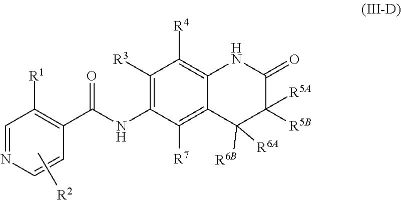

or a pharmaceutically acceptable salt, stereoisomer, tautomer, or deuterated form thereof
wherein:
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or
one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

In embodiments of the compounds of Formula (III), (III-A), (III-B), (III-C), or (III-D), R¹ is —C₁₋₆ haloalkyl, halo, or —NR⁸R⁹.

In embodiments of the compounds of Formula (III), (III-A), (III-B), (III-C), or (III-D), R¹ is —C₁₋₃ haloalkyl, halo, or —NR⁸R⁹.

In embodiments of the compounds of Formula (III), (III-A), (III-B), (III-C), or (III-D), R¹ is —C₁₋₆ haloalkyl.

In embodiments of the compounds of (III), (III-A), (III-B), (III-C), or (III-D), R¹ is —C₁₋₃ haloalkyl.

In embodiments of the compounds of Formula (III), (III-A), (III-B), (III-C), or (III-D), R¹ is —CF₃, —CH₂CF₃, Cl, F, or —NH₂.

In embodiments of the compounds of Formula (III), (III-A), (III-B), (III-C), or (III-D), R¹ is —C₁₋₆ haloalkyl or —NR⁸R⁹.

In embodiments of the compounds of Formula (III), (III-A), (III-B), (III-C), or (III-D), R¹ is —C₁₋₃ haloalkyl or —NR⁸R⁹.

In embodiments of the compounds of Formula (III), (III-A), (III-B), (III-C), or (III-D), R¹ is —CF₃, —CH₂CF₃, or —NH₂.

In embodiments of the compounds of Formula (III), (III-A), (III-B), (III-C), or (III-D), R¹ is —CF₃ or —CH₂CF₃.

In embodiments of the compounds of Formula (III), (III-A), (III-B), (III-C), or (III-D), R¹ is —CF₃.

In embodiments of the compounds of Formula (III), (III-A), (III-B), (III-C), or (III-D), R¹ is —CH₂CF₃.

In embodiments of the compounds of Formula (III), (III-A), (III-B), (III-C), or (III-D), R² is independently halo, —CN, —C₁₋₆ alkyl, —C₁₋₆ alkylene-carbocyclyl, —C₁₋₆ alkylene-heterocyclyl, or —C₁₋₆ haloalkyl.

In embodiments of the compounds of Formula (III), (III-A), (III-B), (III-C), or (III-D), R² is independently —CN, —C₁₋₆ alkyl, —C₁₋₆ alkylene-carbocyclyl, —C₁₋₆ alkylene-heterocyclyl, or —C₁₋₆ haloalkyl.

In embodiments of the compounds of Formula (III), (III-A), (III-B), (III-C), or (III-D), R² is —CN or —C₁₋₆ alkyl.

In embodiments of the compounds of Formula (III), (III-A), (III-B), (III-C), or (III-D), R² is —CN or —C₁₋₃ alkyl.

In embodiments of the compounds of Formula (III), (III-A), (III-B), (III-C), or (III-D), R² is —CN, —CH₃, or —CH₂CH₃.

In embodiments of the compounds of Formula (III), (III-A), (III-B), (III-C), or (III-D), R² is —CN.

In embodiments of the compounds of Formula (III), (III-A), (III-B), (III-C), or (III-D), R² is —C₁₋₆ alkyl.

In embodiments of the compounds of Formula (III), (III-A), (III-B), (III-C), or (III-D), R² is —C₁₋₃ alkyl.

In embodiments of the compounds of Formula (III), (III-A), (III-B), (III-C), or (III-D), R² is —CH₃.

In embodiments of the compounds of Formula (III), (III-A), (III-B), (III-C), or (III-D), R² is —CH₂CH₃.

In embodiments of the compounds of Formula (III), (III-A), (III-B), (III-C), or (III-D), R¹ is —C₁₋₆ haloalkyl and R² is independently —CN or —C₁₋₆ alkyl.

In embodiments of the compounds of Formula (III), (III-A), (III-B), (III-C), or (III-D),), R¹ is —C₁₋₆ haloalkyl and R² is —CN.

In embodiments of the compounds of Formula (III), (III-A), (III-B), (III-C), or (III-D), R¹ is —C₁₋₆ haloalkyl and R² is —C₁₋₆ alkyl.

In embodiments of the compounds of Formula (III), (III-A), (III-B), (III-C), or (III-D), R³ is —H, halo, —C₁₋₆ alkyl, or —C₁₋₆ haloalkyl.

In embodiments of the compounds of Formula (III), (III-A), (III-B), (III-C), or (III-D), R³ is —H or halo.

In embodiments of the compounds of Formula (III), (III-A), (III-B), (III-C), or (III-D), R³ is —H.

In embodiments of the compounds of Formula (III), (III-A), (III-B), (III-C), or (III-D), R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo.

In embodiments of the compounds of Formula (III), (III-A), (III-B), (III-C), or (III-D), R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂.

In embodiments of the compounds of Formula (III), (III-A), (III-B), (III-C), or (III-D), R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl.

In embodiments of the compounds of Formula (III), (III-A), (III-B), (III-C), or (III-D), R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —NR⁸C(O)C₁₋₆ alkyl, —NR⁸R⁹, —C(O)OH, —C(O)OC₁₋₆ alkyl, heterocyclyl, heteroaryl, or carbocyclyl.

In embodiments of the compounds of Formula (III), (III-A), (III-B), (III-C), or (III-D), R⁴ is halo, —CN, —C₁₋₆alkyl, —C₁₋₆ alkyl-CN, —C₁₋₆ haloalkyl, or carbocyclyl.

In embodiments of the compounds of Formula (III), (III-A), (III-B), (III-C), or (III-D), R⁴ is halo or —C₁₋₆alkyl.

In embodiments of the compounds of Formula (III), (III-A), (III-B), (III-C), or (III-D), R⁴ is —C₁₋₆alkyl.

In embodiments of the compounds of Formula (III), (III-A), (III-B), (III-C), or (III-D), is —F or —CH₃.

In embodiments of the compounds of Formula (III), (III-A), (III-B), (III-C), or (III-D), R⁴ is —CH₃.

In embodiments of the compounds of Formula (III), (III-A), (III-B), (III-C), or (III-D), R⁴ is R⁴ is —F, —CN, —OCH₃, —C(O)NH₂,

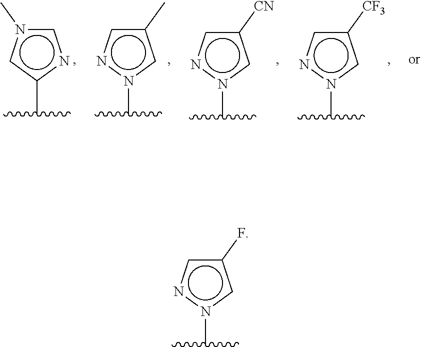

In embodiments of the compounds of Formula (III), (III-A), (III-B), (III-C), or (III-D), R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring.

In embodiments of the compounds of Formula (III), (III-A), (III-B), (III-C), or (III-D), R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 4-8 membered heterocyclic ring.

In embodiments of the compounds of Formula (III), (III-A), (III-B), (III-C), or (III-D), R^{5A}, R^{5B}, R^{6A}, and R^{6B} are each independently —H, halo, or —C₁₋₆ alkyl.

In embodiments of the compounds of Formula (III), (III-A), (III-B), (III-C), or (III-D), R^{5A}, R^{5B}, R^{6A}, and R6B are—H.

In embodiments of the compounds of Formula (III), (III-A), (III-B), (III-C), or (III-D), R⁷ is —H, —C₁₋₆alkyl or halo.

In embodiments of the compounds of Formula (III), (III-A), (III-B), (III-C), or (III-D), R⁷ is —H or halo.

In embodiments of the compounds of Formula (III), (III-A), (III-B), (III-C), or (III-D), R⁷ is —H or—F.

In embodiments of the compounds of Formula (III), (III-A), (III-B), (III-C), or (III-D), R⁷ is —H or —C₁₋₆alkyl.

In embodiments of the compounds of Formula (III), (III-A), (III-B), (III-C), or (III-D), R⁷ is —H.

In embodiments of the compounds of Formula (III), (III-A), (III-B), (III-C), or (III-D), R⁸ and R⁹ are each independently —H, —C₁₋₆ alkyl, aryl or heteroaryl.

In embodiments of the compounds of Formula (III), (III-A), (III-B), (III-C), or (III-D), R⁸ and R⁹ are each independently —H or —C₁₋₆ alkyl.

In embodiments of the compounds of Formula (III), (III-A), (III-B), (III-C), or (III-D), R⁸ is —H or —C₁₋₆alkyl.

In embodiments of the compounds of Formula (III), (III-A), (III-B), (III-C), or (III-D), R⁸ is —H.

In embodiments of the compounds of Formula (III), (III-A), (III-B), (III-C), or (III-D), R⁸ is —C₁₋₆alkyl.

In embodiments of the compounds of Formula (III), (III-A), (III-B), (III-C), or (III-D), R⁹ is —H or —C₁₋₆alkyl.

In embodiments of the compounds of Formula (III), (III-A), (III-B), (III-C), or (III-D), R⁹ is —H.

In embodiments of the compounds of Formula (III), (III-A), (III-B), (III-C), or (III-D), R⁹ is —C₁₋₆alkyl.

In embodiments of the compounds of Formula (III), (III-A), (III-B), (III-C), or (III-D), R⁸ and R⁹ are —H.

In embodiments, provided herein is a compound of Formula (III), (III-A), (III-B), (III-C), or (III-D), or a pharmaceutically acceptable salt or deuterated form thereof.

In embodiments, provided herein is a compound of Formula (III), (III-A), (III-B), (III-C), or (III-D), or a pharmaceutically acceptable salt thereof.

In embodiments, provided herein is a compound of Formula (III), (III-A), (III-B), (III-C), or (III-D).

### Compounds of Formula (IV)

In embodiments, provided herein is a compound of Formula (IV′):

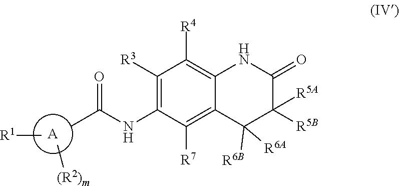

or a pharmaceutically acceptable salt, stereoisomer, tautomer, or deuterated form thereof;
wherein:
ring A is heteroaryl or aryl;
R¹ is —C₁₋₆ haloalkyl, -halo or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
m is 0, 1 or 2;
R³ is —H, -halo, —C₁₋₆ alkyl, or —C₁₋₆ haloalkyl;
R⁴ is —F, —C₁₋₆alkyl, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H or halo; and
R⁸ and R⁹ are each independently —H, —C₁₋₆alkyl, aryl, or heteroaryl.

In embodiments of the compounds of Formula (IV′), the compound is not:
(i) 5-Isothiazolecarboxamide, 3-methyl-N-(1,2,3,4-tetrahydro-8-methyl-2-oxo-6-quinolinyl)-4-(2,2,2-trifluoroethyl)-;
(ii) 5-Isothiazolecarboxamide, 3-cyano-N-(1,2,3,4-tetrahydro-8-methyl-2-oxo-6-quinolinyl)-4-(2,2,2-trifluoroethyl)-;
(iii) 4-Pyridinecarboxamide, 2-chloro-5-ethyl-N-(1,2,3,4-tetrahydro-8-methyl-2-oxo-6-quinolinyl)-;
(iv) 5H-Cyclopenta[b]pyridine-4-carboxamide, 6,6-difluoro-6,7-dihydro-N-(1,2,3,4-tetrahydro-8-methyl-2-oxo-6-quinolinyl)-; or
(v) 4-Quinolinecarboxamide, 6,6-difluoro-5,6,7,8-tetrahydro-N-(1,2,3,4-tetrahydro-8-methyl-2-oxo-6-quinolinyl)-.

In embodiments, provided herein is a compound of Formula (IV):

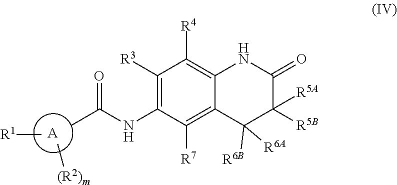

or a pharmaceutically acceptable salt, stereoisomer, tautomer, or deuterated form thereof;
wherein:
ring A is heteroaryl or aryl;
R¹ is —C₁₋₆ haloalkyl, halo or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
mis 0, 1 or 2;
R³ is —H, —C₁₋₆ alkyl, or —C₁₋₆ haloalkyl;
R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl;

R^{5A}, R^{5B}, R^{6A}, and R^{6B} are each independently —H, halo, or —C₁₋₆ alkyl; or one of RSA and R^{5B} is joined with one of R^{6A} and R^{6B} to form an optionally substituted C₃₋₈ carbocyclic ring, or an optionally substituted 3-8 membered heterocyclic ring; and
R⁷ is —H or halo; and
R⁸ and R⁹ are each independently —H, —C₁₋₆alkyl, aryl, or heteroaryl.

In embodiments of the compounds of Formula (IV), the compound is not:
(i) 5-Isothiazolecarboxamide, 3-methyl-N-(1,2,3,4-tetrahydro-8-methyl-2-oxo-6-quinolinyl)-4-(2,2,2-trifluoroethyl)-;
(ii) 5-Isothiazolecarboxamide, 3-cyano-N-(1,2,3,4-tetrahydro-8-methyl-2-oxo-6-quinolinyl)-4-(2,2,2-trifluoroethyl)-;
(iii) 4-Pyridinecarboxamide, 2-chloro-5-ethyl-N-(1,2,3,4-tetrahydro-8-methyl-2-oxo-6-quinolinyl)-;
(iv) 5H-Cyclopenta[b]pyridine-4-carboxamide, 6,6-difluoro-6,7-dihydro-N-(1,2,3,4-tetrahydro-8-methyl-2-oxo-6-quinolinyl)-; or
(v) 4-Quinolinecarboxamide, 6,6-difluoro-5,6,7,8-tetrahydro-N-(1,2,3,4-tetrahydro-8-methyl-2-oxo-6-quinolinyl)-.

In embodiments of the compounds of Formula (IV′) or (IV), ring A is heteroaryl.

In embodiments of the compounds of Formula (IV′) or (IV), ring A is heteroaryl comprising at least one nitrogen atom.

In embodiments of the compounds of Formula (IV′) or (IV), ring A is a monocyclic heteroaryl.

In embodiments of the compounds of Formula (IV′) or (IV), ring A is a monocyclic heteroaryl comprising at least one nitrogen atom.

In embodiments of the compounds of Formula (IV′) or (IV), ring A is a 5- or 6-membered heteroaryl.

In embodiments of the compounds of Formula (IV′) or (IV), ring A is a 6-membered heteroaryl.

In embodiments of the compounds of Formula (IV′) or (IV), ring A is 6-membered heteroaryl comprising at least one nitrogen atom.

In embodiments of the compounds of Formula (IV′) or (IV), ring A is 6-membered heteroaryl comprising one or two nitrogen atoms.

In embodiments of the compounds of Formula (IV′) or (IV), ring A is not an isothiazole.

In embodiments of the compounds of Formula (IV′) or (IV), ring A is a heteroaryl having one or more heteroatoms independently selected from N and O. In embodiments, ring A is a monocyclic heteroaryl having one or more heteroatoms independently selected from N and O. In embodiments, ring A is a 5- or 6-membered heteroaryl having one or more heteroatoms independently selected from N and O. In embodiments, ring A is a 6-membered heteroaryl having one or more heteroatoms independently selected from N and O.

In embodiments of the compounds of Formula (IV′) or (IV), ring A is a heteroaryl having one or two heteroatoms independently selected from N and O. In embodiments, ring A is a monocyclic heteroaryl having one or two heteroatoms independently selected from N and O. In embodiments, ring A is a 5- or 6-membered heteroaryl having one or two heteroatoms independently selected from N and O. In embodiments, ring A is a 6-membered heteroaryl having one or two heteroatoms independently selected from N and O.

In embodiments of the compounds of Formula (IV′) or (IV), ring A is a heteroaryl having one or two N atoms. In embodiments, ring A is a monocyclic heteroaryl having one or two N atoms. In embodiments, ring A is a 5- or 6-membered heteroaryl having one or two N atoms. In embodiments ring A is a 6-membered heteroaryl having one or two N atoms.

In embodiments of the compounds of Formula (IV′) or (IV), ring A is pyridyl.

In embodiments of the compounds of Formula (IV′) or (IV), ring A is aryl.

In embodiments of the compounds of Formula (IV′) or (IV), ring A is a phenyl.

In embodiments, provided herein is a compound of Formula (IV-A):

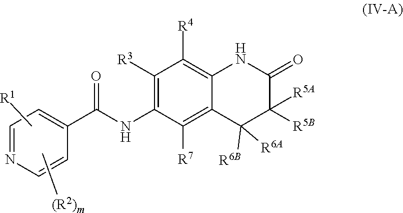

or a pharmaceutically acceptable salt, stereoisomer, tautomer, or deuterated form thereof;
wherein:

R¹ is —C₁₋₆ haloalkyl, halo or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
m is 0, 1 or 2;
R³ is —H, —C₁₋₆ alkyl, or —C₁₋₆ haloalkyl;
R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H or halo; and
R⁸ and R⁹ are each independently —H, —C₁₋₆alkyl, aryl, or heteroaryl.

In embodiments of the compounds of Formula (IV-A), the compound is not
(i) 4-Pyridinecarboxamide, 2-chloro-5-ethyl-N-(1,2,3,4-tetrahydro-8-methyl-2-oxo-6-quinolinyl)-;
(ii) 5H-Cyclopenta[b]pyridine-4-carboxamide, 6,6-difluoro-6,7-dihydro-N-(1,2,3,4-tetrahydro-8-methyl-2-oxo-6-quinolinyl)-; or
(iii) 4-Quinolinecarboxamide, 6,6-difluoro-5,6,7,8-tetrahydro-N-(1,2,3,4-tetrahydro-8-methyl-2-oxo-6-quinolinyl)-.

In embodiments, provided herein is a compound of Formula (IV-B):

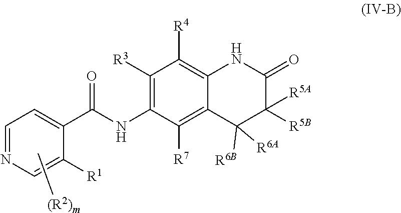

or a pharmaceutically acceptable salt, stereoisomer, tautomer, or deuterated form thereof;
wherein:
R¹ is —C₁₋₆ haloalkyl, halo or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
m is 0, 1 or 2;
R³ is —H, —C₁₋₆ alkyl, or —C₁₋₆ haloalkyl;
R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl;
R^{5A}, R^{5B}, R^{6A}, and R^{6B} are each independently —H, halo, or —C₁₋₆ alkyl; or one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H or halo; and
R⁸ and R⁹ are each independently —H, —C₁₋₆alkyl, aryl, or heteroaryl.

In embodiments of the compounds of Formula (IV′), (IV), (IV-A), or (IV-B), R¹ is —C₁₋₆ haloalkyl, halo or —NR⁸R⁹.

In embodiments of the compounds of Formula (IV′), (IV), (IV-A), or (IV-B), R¹ is —C₁₋₆ haloalkyl or —NR⁸R⁹.

In embodiments of the compounds of Formula (IV′), (IV), (IV-A), or (IV-B), R¹ is —C₁₋₃ haloalkyl or —NR⁸R⁹.

In embodiments of the compounds of Formula (IV′), (IV), (IV-A), or (IV-B), R¹ is —CF₃, —CH₂CF₃, or —NH₂.

In embodiments of the compounds of Formula (IV′), (IV), (IV-A), or (IV-B), R¹ is —C₁₋₆ haloalkyl.

In embodiments of the compounds of Formula (IV′), (IV), (IV-A), or (IV-B), R¹ is —C₁₋₃ haloalkyl.

In embodiments of the compounds of Formula (IV′), (IV), (IV-A), or (IV-B), R¹ is R¹ is —CH₂CF₃.

In embodiments of the compounds of Formula (IV′), (IV), (IV-A), or (IV-B), m is 0, 1 or 2.

In embodiments of the compounds of Formula (IV′), (IV), (IV-A), or (IV-B), m is 0.

In embodiments of the compounds of Formula (IV′), (IV), (IV-A), or (IV-B), m is 1.

In embodiments of the compounds of Formula (IV′), (IV), (IV-A), or (IV-B), m is 2.

In embodiments of the compounds of Formula (IV′), (IV), (IV-A), or (IV-B), R² is independently halo, —CN, —C₁₋₆ alkyl, —C₁₋₆ alkylene-carbocyclyl, —C₁₋₆ alkylene-heterocyclyl, or—C₁₋₆ haloalkyl.

In embodiments of the compounds of Formula (IV′), (IV), (IV-A), or (IV-B), R² is independently halo, —CN, —C₁₋₆ alkyl, or —C₁₋₆ haloalkyl.

In embodiments of the compounds of Formula (IV′), (IV), (IV-A), or (IV-B), R³ is —H, —C₁₋₆ alkyl, or —C₁₋₆ haloalkyl.

In embodiments of the compounds of Formula (IV′), (IV), (IV-A), or (IV-B), R³ is —H.

In embodiments of the compounds of Formula (IV′), (IV), (IV-A), or (IV-B), R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl.

In embodiments of the compounds of Formula (IV′), (IV), (IV-A), or (IV-B), R⁴ is —F, —CN, —OH, —C₁₋₆ alkyl-CN, —OC₁₋₆ alkyl, —C(O)NR⁸R⁹, —NR⁸C(O)C₁₋₆ alkyl, —NR⁸R⁹, —C(O)OH, —C(O)OC₁₋₆ alkyl, heterocyclyl, or heteroaryl.

In embodiments of the compounds of Formula (IV′), (IV), (IV-A), or (IV-B), R⁴ is —F, —CN, OC₁₋₆ alkyl, or —C(O)N(R⁸R⁹)₂.

In embodiments of the compounds of Formula (IV′), (IV), (IV-A), or (IV-B), R⁴ is a 5-6 membered heteroaryl. In embodiments, the heteroaryl is a 5-membered heteroaryl containing two nitrogen atoms.

In embodiments of the compounds of Formula (IV′), (IV), (IV-A), or (IV-B), R⁴ is —F, —CN, —OCH₃, —C(O)NH₂,

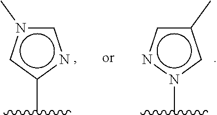

In embodiments of the compounds of Formula (IV′), (IV), (IV-A), or (IV-B), R⁴ is —F, —CN, —OCH₃, —C(O)NH₂,

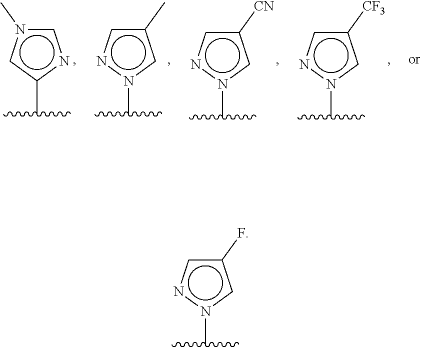

In embodiments of the compounds of Formula (IV′), (IV), (IV-A), or (IV-B), R^{5A}, R^{5B}, R^{6A}, and R^{6B} are each independently —H, halo, or —C₁₋₆ alkyl, or one of R^{5A} and R^{5B} is joined with one of R^{6A} and R^{6B} to form an optionally substituted C₃₋₈ carbocyclic ring, or an optionally substituted 3-8 membered heterocyclic ring.

In embodiments of the compounds of Formula (IV′), (IV), (IV-A), or (IV-B), R^{5A}, R^{5B}, R^{6A}, and R^{6B} are each independently —H, halo, or —C₁₋₆ alkyl, or one of R^{5A} and R^{5B} is joined with one of R^{6A} and R^{6B} to form an optionally substituted C₃₋₈ carbocyclic ring, or an optionally substituted 4-8 membered heterocyclic ring.

In embodiments of the compounds of Formula (IV′), (IV), (IV-A), or (IV-B), R^{5A}, R^{5B}, R^{6A}, and R^{6B} are each independently —H, halo, or —C₁₋₆ alkyl.

In embodiments of the compounds of Formula (IV′), (IV), (IV-A), or (IV-B), R^{5A}, R^{5B}, R^{6A}, and R^{6B} are—H.

In embodiments of the compounds of Formula (IV′), (IV), (IV-A), or (IV-B), R⁷ is —H, —C₁₋₆alkyl or halo.

In embodiments of the compounds of Formula (IV′), (IV), (IV-A), or (IV-B), R⁷ is —H or halo.

In embodiments of the compounds of Formula (IV′), (IV), (IV-A), or (IV-B), R⁷ is —H or —F.

In embodiments of the compounds of Formula (IV′), (IV), (IV-A), or (IV-B), R⁷ is —H or —C₁₋₆alkyl.

In embodiments of the compounds of Formula (IV′), (IV), (IV-A), or (IV-B), R⁷ is —H.

In embodiments of the compounds of Formula (IV′), (IV), (IV-A), or (IV-B), R⁸ and R⁹ are each independently —H, —C₁₋₆ alkyl, aryl or heteroaryl.

In embodiments of the compounds of Formula (IV′), (IV), (IV-A), or (IV-B), R⁸ and R⁹ are each independently —H or —C₁₋₆ alkyl.

In embodiments of the compounds of Formula (IV′), (IV), (IV-A), or (IV-B), R⁸ is —H or —C₁₋₆alkyl.

In embodiments of the compounds of Formula (IV′), (IV), (IV-A), or (IV-B), R⁸ is —H.

In embodiments of the compounds of Formula (IV′), (IV), (IV-A), or (IV-B), R⁸ is —C₁₋₆alkyl.

In embodiments of the compounds of Formula (IV′), (IV), (IV-A), or (IV-B), R⁹ is —H or —C₁₋₆alkyl.

In embodiments of the compounds of Formula (IV′), (IV), (IV-A), or (IV-B), R⁹ is —H.

In embodiments of the compounds of Formula (IV′), (IV), (IV-A), or (IV-B), R⁹ is —C₁₋₆alkyl.

In embodiments of the compounds of Formula (IV′), (IV), (IV-A), or (IV-B), R⁸ and R⁹ are —H.

In embodiments, provided herein is a compound of Formula (IV′), (IV), (IV-A), or (IV-B), or a pharmaceutically acceptable salt or deuterated form thereof.

In embodiments, provided herein is a compound of Formula (IV′), (IV), (IV-A), or (IV-B), or a pharmaceutically acceptable salt thereof.

In embodiments, provided herein is a compound of Formula (IV′), (IV), (IV-A), or (IV-B).

In embodiments, provided herein is one or more compounds of Table 1, or a pharmaceutically acceptable salt, stereoisomer, tautomer, or deuterated form thereof.

In embodiments, provided herein is one or more compounds of Table 1, or a pharmaceutically acceptable salt or deuterated form thereof.

In embodiments, provided herein is one or more compounds of Table 1, or a pharmaceutically acceptable salt thereof.

In embodiments, provided herein is one or more compounds of Table 1.

TABLE 1
Compounds

| No. | Structure |
|---|---|
| 1 | 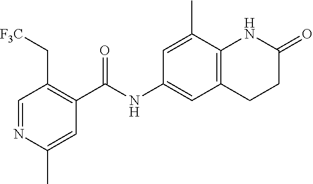 [SMILES: Cc1cc(C(=O)Nc2cc(C)c3c(c2)CCC(=O)N3)c(CC(F)(F)F)cn1] |
| 2 | 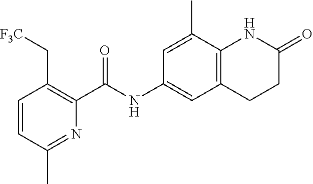 [SMILES: Cc1ccc(CC(F)(F)F)c(C(=O)Nc2cc(C)c3c(c2)CCC(=O)N3)n1] |
| 3 | 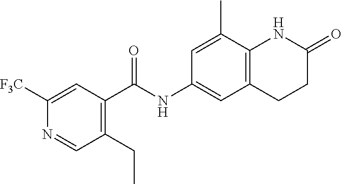 [SMILES: CCc1cnc(C(F)(F)F)cc1C(=O)Nc1cc(C)c2c(c1)CCC(=O)N2] |
| 4 | 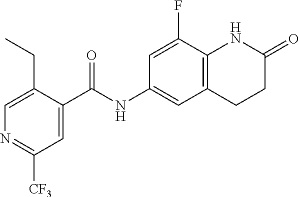 [SMILES: CCc1cnc(C(F)(F)F)cc1C(=O)Nc1cc(F)c2c(c1)CCC(=O)N2] |
| 5 | 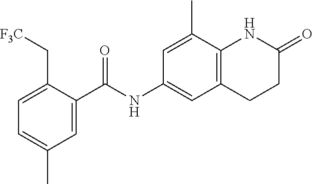 [SMILES: Cc1ccc(CC(F)(F)F)c(C(=O)Nc2cc(C)c3c(c2)CCC(=O)N3)c1] |
| 6 | 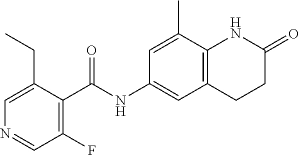 [SMILES: CCc1cncc(F)c1C(=O)Nc1cc(C)c2c(c1)CCC(=O)N2] |
| 7 | 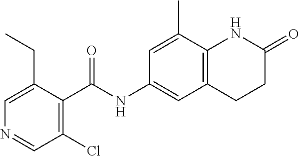 [SMILES: CCc1cncc(Cl)c1C(=O)Nc1cc(C)c2c(c1)CCC(=O)N2] |
| 8 | 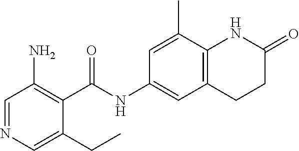 [SMILES: CCc1cncc(N)c1C(=O)Nc1cc(C)c2c(c1)CCC(=O)N2] |
| 9 | 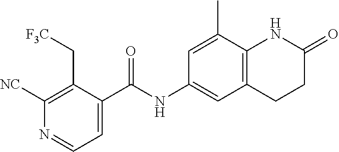 [SMILES: Cc1cc(NC(=O)c2ccnc(C#N)c2CC(F)(F)F)cc2c1NC(=O)CC2] |
| 10 | 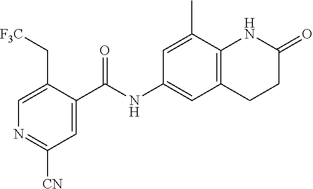 [SMILES: Cc1cc(NC(=O)c2cc(C#N)ncc2CC(F)(F)F)cc2c1NC(=O)CC2] |
| 11 | 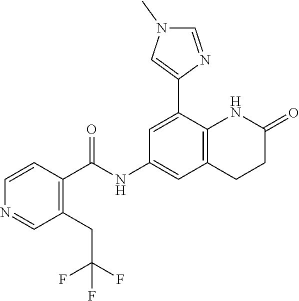 [SMILES: Cn1cnc(-c2cc(NC(=O)c3ccncc3CC(F)(F)F)cc3c2NC(=O)CC3)c1] |
| 12 | 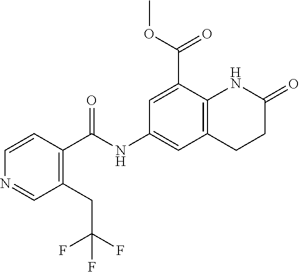 [SMILES: COC(=O)c1cc(NC(=O)c2ccncc2CC(F)(F)F)cc2c1NC(=O)CC2] |
| 13 | 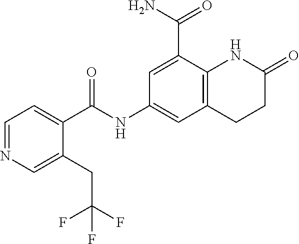 [SMILES: NC(=O)c1cc(NC(=O)c2ccncc2CC(F)(F)F)cc2c1NC(=O)CC2] |
| 14 | 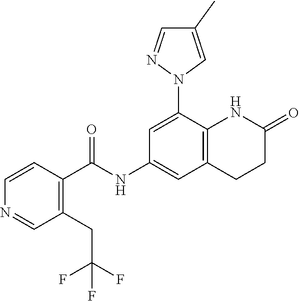 [SMILES: Cc1cnn(-c2cc(NC(=O)c3ccncc3CC(F)(F)F)cc3c2NC(=O)CC3)c1] |
| 15 | 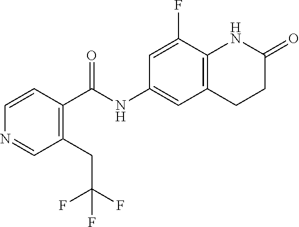 [SMILES: O=C1CCc2cc(NC(=O)c3ccncc3CC(F)(F)F)cc(F)c2N1] |
| 16 | 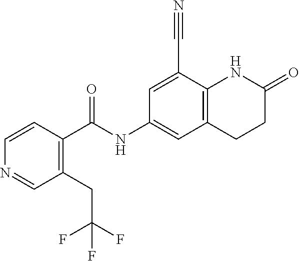 [SMILES: N#Cc1cc(NC(=O)c2ccncc2CC(F)(F)F)cc2c1NC(=O)CC2] |
| 17 | 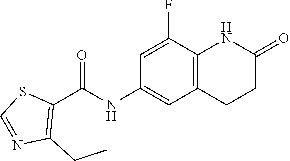 [SMILES: CCc1ncsc1C(=O)Nc1cc(F)c2c(c1)CCC(=O)N2] |
| 18 | 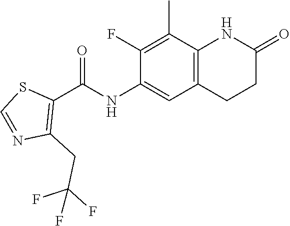 [SMILES: Cc1c(F)c(NC(=O)c2scnc2CC(F)(F)F)cc2c1NC(=O)CC2] |
| 19 | 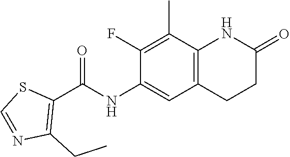 [SMILES: CCc1ncsc1C(=O)Nc1cc2c(c(C)c1F)NC(=O)CC2] |
| 20 | 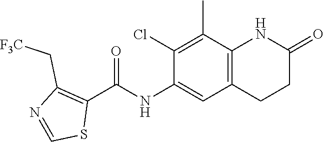 [SMILES: Cc1c(Cl)c(NC(=O)c2scnc2CC(F)(F)F)cc2c1NC(=O)CC2] |
| 21 | 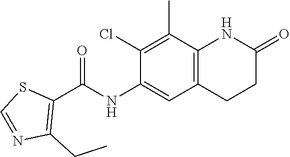 [SMILES: CCc1ncsc1C(=O)Nc1cc2c(c(C)c1Cl)NC(=O)CC2] |
| 22 | 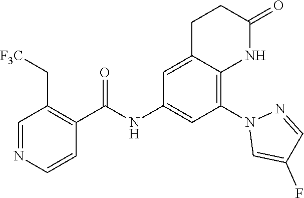 [SMILES: O=C1CCc2cc(NC(=O)c3ccncc3CC(F)(F)F)cc(-n3cc(F)cn3)c2N1] |
| 23 | 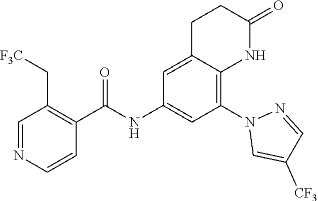 [SMILES: O=C1CCc2cc(NC(=O)c3ccncc3CC(F)(F)F)cc(-n3cc(C(F)(F)F)cn3)c2N1] |
| 24 | 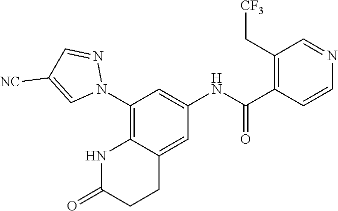 [SMILES: N#Cc1cnn(-c2cc(NC(=O)c3ccncc3CC(F)(F)F)cc3c2NC(=O)CC3)c1] |
| 25 | 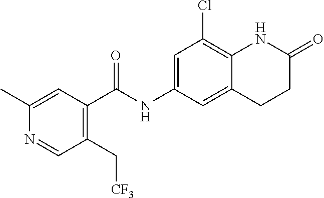 [SMILES: Cc1cc(C(=O)Nc2cc(Cl)c3c(c2)CCC(=O)N3)c(CC(F)(F)F)cn1] |
| 26 | 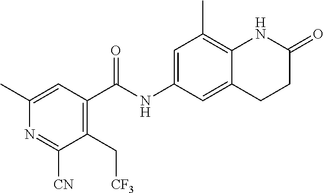 [SMILES: Cc1cc(C(=O)Nc2cc(C)c3c(c2)CCC(=O)N3)c(CC(F)(F)F)c(C#N)n1] |

### Compositions

In embodiments, the present disclosure provides pharmaceutical compositions for modulating (e.g., inhibiting) aldehyde dehydrogenase (e.g., ALDH1a3) in a subject. In some embodiments, a pharmaceutical composition comprises one or more compounds of the present disclosure (e.g., a compound of Formula (A), (A-I), (I), (I-A), (I-B), (I-C), (I-D), (II), (II-A), (II-B), (III), (III-A), (III-B), (III-C), (III-D), (IV′), (IV), (IV-A), or (IV-B).
or Table 1 or a pharmaceutically acceptable salt, stereoisomer, tautomer, or deuterated form thereof).

In embodiments, a pharmaceutical composition comprises one or more compounds of Formula (A), (A-I), (I), (I-A), (I-B), (I-C), (I-D), (II), (II-A), (II-B), (III), (III-A), (III-B), (III-C), or (III-D), (IV′), (IV), (IV-A), or (IV-B).
or Table 1 or a pharmaceutically acceptable salt or deuterated form thereof.

In embodiments, a pharmaceutical composition comprises one or more compounds of Formula (A), (A-I), (I), (I-A), (I-B), (I-C), (I-D), (II), (II-A), (II-B), (III), (III-A), (III-B), (III-C), or (III-D), (IV′), (IV), (IV-A), (IV-B).
or Table 1 or a pharmaceutically acceptable salt thereof.

In embodiments of the present disclosure, a pharmaceutical composition comprises a therapeutically effective amount of one or more compounds of the present disclosure (e.g., a compound of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt, stereoisomer, tautomer, or deuterated form thereof) and a pharmaceutically acceptable carrier.

In embodiments, the present disclosure provides a pharmaceutical composition comprising one or more compounds of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt or deuterated form thereof, and a pharmaceutically acceptable carrier.

In embodiments, the present disclosure provides a pharmaceutical composition comprising one or more compounds of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt thereof and a pharmaceutically acceptable carrier.

In embodiments, a pharmaceutical composition, as described herein, comprises one or more compounds selected from Table 1, or a pharmaceutically acceptable salt, stereoisomer, tautomer, or deuterated form thereof and a pharmaceutically acceptable carrier.

In embodiments, a pharmaceutical composition, as described herein, comprises one or more compounds selected from Table 1, or a pharmaceutically acceptable salt or deuterated form thereof, and a pharmaceutically acceptable carrier.

In embodiments, a pharmaceutical composition, as described herein, comprises one or more compounds selected from Table 1, or a pharmaceutically acceptable salt thereof, and a pharmaceutically acceptable carrier.

In embodiments of the present disclosure, a pharmaceutical composition comprising one or more compounds of the present disclosure (e.g., a compound of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt, stereoisomer, tautomer, or deuterated form thereof), and a pharmaceutically acceptable excipient or adjuvant is provided. The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, transdermally, buccally, sublingually, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion.

In some embodiments, the pharmaceutical composition is formulated for inhalation. The inhalable formulations can be, for example, formulated as a nasal spray, dry powder, or an aerosol administrable through a metered-dose inhaler.

Compounds of the present disclosure can be used alone, in combination with each other, or in combination with one or more additional therapeutic agents, When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

In some embodiments, compounds of the present disclosure can also be used for treating type 2 diabetes in combination with one or more additional therapeutic agents useful for treating type 2 diabetes, e.g., metformin, recombinant insulin, liraglutide, semaglutide, empagliflozin etc.

Generally, the compounds of the present disclosure are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

### Therapeutic Use

The compounds of the present disclosure find use in any number of methods. For example, in embodiments, the compounds of the present disclosure are useful in methods for inhibiting ALDH1a3. Accordingly, in some embodiments, the present disclosure provides the use of any one of the foregoing compounds of (e.g., a compound of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt, stereoisomer, tautomer, or deuterated form thereof) for inhibiting ALDH1a3 activity. For example, in some embodiments, inhibiting ALDH1a3 activity is in a mammalian cell. In embodiments, inhibiting ALDH1a3 activity can be in a subject in need thereof (e.g., a mammalian subject, such as a human) and for treatment of any of the described conditions or diseases.

In some embodiments, inhibiting ALDH1a3 activity is binding aldehyde dehydrogenase e.g., ALDH1a3. In embodiments, ALDH1a3 is selectively inhibited. In embodiments, the compound of the present disclosure (e.g., a compound of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt, stereoisomer, tautomer, or deuterated form thereof) exhibits selectivity for ALDH1A3 over ALDH1A2. In embodiments, the compound of the present disclosure (e.g., a compound of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt, stereoisomer, tautomer, or deuterated form thereof) is an ALDH1A3 isoform selective inhibitor.

In some embodiments, the present disclosure provides methods of treating a disease or disorder that is treatable by administration of an aldehyde dehydrogenase inhibitor (e.g., an aldehyde dehydrogenase isoform 1a3 (ALDH1a3), the method comprising administering a therapeutically effective amount of one or more compounds of the present disclosure (e.g., a compound of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt, stereoisomer, tautomer, or deuterated form thereof).

In some embodiments, the disease or disorder is associated with aldehyde dehydrogenase (e.g., an aldehyde dehydrogenase isoform 1a3 (ALDH1a3)). In embodiments, the disease or disorder is associated with aldehyde dehydrogenase isoform 1a3 (ALDH1a3) in the subject. For example, in some embodiments, the disease or disorder is type 2 diabetes. other embodiments, the disease or disorder is a lipid disorder such as hyperlipidemia or dyslipidemia, nonalcoholic fatty liver disease (NAFLD), nonalcoholic steatohepatitis (NASH), Metabolic Dysfunction-Associated Steatohepatitis (MASH), hypercholesterolemia, hypertriglyceridemia, familial cholesterolemia, familial triglyceridemia. In other embodiments, the disorder is a cardiovascular disorder, such as pulmonary arterial hypertension, chronic heart failure, neointimal hyperplasia, Atherosclerotic Cardiovascular Disease (ASCVD), heart failure with preserved ejection fraction, ischemic heart disease or coronary artery disease. In other embodiments, the disease is a kidney disease caused by or associated with type 2 diabetes or cardiometabolic syndrome. In other embodiments, the disease is sarcopenia or bone density loss due to cardiometabolic disease. In embodiments, the disease or disorder is cardiometabolic syndrome. In embodiments, the disease or disorder is a diabetic kidney disease. In embodiments, the disease or disorder is a lipid disorder, such as hyperlipidemia or dyslipidemia. In embodiments, the disease or disorder is muscle inflammation. In embodiments, the disease or disorder is sarcopenia or bone density loss. In embodiments, the disease or disorder is non-alcoholic fatty liver disease (NAFLD). In embodiments, the disease or disorder is non-alcoholic steatohepatitis (NASH). In embodiments, the disease or disorder is metabolic dysfunction-associated steatohepatitis (MASH). In embodiments, the disease or disorder is hypercholesterolemia. In embodiments, the disease or disorder is hypertriglyceridemia. In embodiments, the disease or disorder is familial cholesterolemia. In embodiments, the disease or disorder is familial triglyceridemia. In embodiments, the disease or disorder is a cardiovascular disorder, such as pulmonary arterial hypertension, chronic heart failure, neointimal hyperplasia, Atherosclerotic Cardiovascular Disease (ASCVD), heart failure with preserved ejection fraction, ischemic heart disease or coronary artery disease. In other embodiments, the disease or disorder is a proliferative disease or disorder. In embodiments, the disease or disorder is cancer such as breast cancer, lung cancer, gastric cancer, prostate cancer, sarcoma, melanoma, leukemia, lymphoma, thyroid cancer.

In embodiments, the present disclosure provides methods of treating a cardiometabolic syndrome, a diabetic kidney disease, a lipid disorder (e.g., hyperlipidemia or dyslipidemia), muscle inflammation, sarcopenia or bone density loss, non-alcoholic fatty liver disease (NAFLD), non-alcoholic steatohepatitis (NASH), metabolic dysfunction-associated steatohepatitis (MASH), hypercholesterolemia, hypertriglyceridemia, familial cholesterolemia, familial triglyceridemia, or a cardiovascular disorder (e.g., pulmonary arterial hypertension, chronic heart failure, neointimal hyperplasia, Atherosclerotic Cardiovascular Disease (ASCVD), heart failure with preserved ejection fraction, ischemic heart disease or coronary artery disease) comprising administering a compound (e.g., a compound of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt, stereoisomer, tautomer, or deuterated form thereof) or pharmaceutical composition of the present disclosure.

In embodiments, the present disclosure provides methods for the treatment of a cardiometabolic syndrome, a diabetic kidney disease, a lipid disorder (e.g., hyperlipidemia or dyslipidemia), muscle inflammation, sarcopenia or bone density loss, non-alcoholic fatty liver disease (NAFLD), non-alcoholic steatohepatitis (NASH), metabolic dysfunction-associated steatohepatitis (MASH), hypercholesterolemia, hypertriglyceridemia, familial cholesterolemia, familial triglyceridemia, or a cardiovascular disorder (e.g., pulmonary arterial hypertension, chronic heart failure, neointimal hyperplasia, Atherosclerotic Cardiovascular Disease (ASCVD), heart failure with preserved ejection fraction, ischemic heart disease or coronary artery disease) in a subject in need thereof, comprising administering one or more compounds disclosed in US 2021/0155602, US 2023/0128402, US 2024/0132469 or PCT/US2023/077659, each of which is incorporated by reference herein.

In embodiments, the subject suffers from a disease or disorder associated with aldehyde dehydrogenase, for example, a disease or disorder associated with aldehyde dehydrogenase isoform 1a3 (ALDH1a3). In embodiments, the subject suffers from a disease or disorder associated with aldehyde dehydrogenase isoform 1a3 (ALDH1a3). In embodiments, the subject suffers from type 2 diabetes.

In embodiments, compounds of the present disclosure (e.g., a compound of Formula (A), (A-I), (I), (I-A), (I-B), (I-C), (I-D), (II), (II-A), (II-B), (III), (III-A), (III-B), (III-C), (III-D), (IV′), (IV), (IV-A), (IV-B), or Table 1 or a pharmaceutically acceptable salt, stereoisomer, tautomer, or deuterated form thereof) are used for the treatment of diseases or disorders that are associated with aldehyde dehydrogenase, such as a disease or disorder associated with ALDH1a3. In embodiments, compounds of the present disclosure are used for the treatment of diseases or disorders that are associated with ALDH1a3.

In embodiments, compounds of the present disclosure are used for the treatment of diseases or disorders that are associated with ALDH1a3.

In embodiments, compounds of the present disclosure are used for the treatment and/or prophylaxis of type 2 diabetes. In embodiments, treating type 2 diabetes comprises treating type 2 diabetes-associated dyslipidemia. In embodiments prophylaxis of type 2 diabetes comprises prophylaxis of type 2 diabetes-associated dyslipidemia.

In embodiments, the compounds of the present disclosure are used for the treatment of one or more of the diseases or disorders disclosed herein. In embodiments, the compounds of the present disclosure are used for the prophylaxis (or prevention) of one or more of the diseases or disorders disclosed herein.

As discussed herein, metabolic diseases such as type 2 diabetes are associated with a pathology driven by ALDH1a3 activities. In some embodiments, the method further comprises administering to the subject an effective amount of an additional anti-metabolic diseases agents, such as anti-type 2 diabetes agent. Suitable additional anti-metabolic diseases agents include without limitation an incretin mimic, recombinant insulin, a biguanide, SGLT2 inhibitors, a therapeutic antibody, etc. Any of the known Type 2 Diabetes treatments can be used in combination with the compounds of the present disclosure, for example, for treating Type 2 Diabetes (e.g., described herein) or treating or preventing other metabolic syndromes.

In embodiments, the compound of the present disclosure recited in the methods herein is a compound of the present disclosure having an IC50 value of less than 250 nM (e.g., in particular embodiments, less than 100 nM, such as about 1-100 nM, about 10-100 nM, about 10-50 nM, about 20-100 nM, about 20-50 nM, etc.) in inhibiting ALDH1a3 when measured by the method described herein in example 16.

In embodiments, the compound of the present disclosure recited in the methods herein has at least about a 2-fold selectivity for inhibiting ALDH1a3 over ALDH1a2. In embodiments, the compound has at least about a 3-fold selectivity for inhibiting ALDH1a3 over ALDH1a2. In embodiments, the compound has at least about a 4-fold selectivity for inhibiting ALDH1a3 over ALDH1a2. In embodiments, the compound has at least about a 5-fold selectivity for inhibiting ALDH1a3 over ALDH1a2. In embodiments, the compound has at least about a 6-fold selectivity for inhibiting ALDH1a3 over ALDH1a2. In embodiments, the compound has at least about a 7-fold selectivity for inhibiting ALDH1a3 over ALDH1a2. In embodiments, the compound has at least about an 8-fold selectivity for inhibiting ALDH1a3 over ALDH1a2. In embodiments, the compound has at least about a 9-fold selectivity for inhibiting ALDH1a3 over ALDH1a2. In embodiments, the compound has at least about a 10-fold selectivity for inhibiting ALDH1a3 over ALDH1a2. In embodiments, the compound has at least about a 11-fold selectivity for inhibiting ALDH1a3 over ALDH1a2. In embodiments, the compound has at least about a 12-fold selectivity for inhibiting ALDH1a3 over ALDH1a2. In embodiments, inhibiting ALDH1a3 or ALDH1a2 is measured by the methods described herein in example 16.

### NUMBERED EMBODIMENTS #1

1. A method of treating or preventing a disease or disorder in a subject in need thereof, the method comprising administering to the subject an effective amount of:
(i) a compound of Formula (A) or a pharmaceutically acceptable salt, stereoisomer, tautomer, or deuterated form thereof; or
(ii) a pharmaceutical composition comprising a compound of Formula (A) or a pharmaceutically acceptable salt, stereoisomer, tautomer, or deuterated form thereof, and a pharmaceutically acceptable carrier:

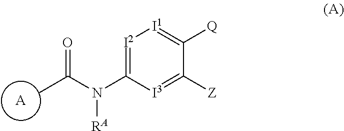

wherein:
ring A is an optionally substituted heteroaryl, an optionally substituted heterocyclyl or an optionally substituted aryl;
R^{A} is —H or —C₁₋₆alkyl;
I¹ is CR⁴ or N;
I² is CR³ or N;
I³ is CR⁷ or N;
R³ is —H, halo, —C₁₋₆ alkyl, or —C₁₋₆ haloalkyl;
R⁴ is —H, halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo;
Q and Z are joined to form a heterocyclyl and substituted with oxo; and wherein the heterocycle may be further optionally substituted;
R⁷ is —H, —C₁₋₆alkyl or halo;
R⁸ and R⁹ are each independently —H, —C₁₋₆ alkyl, aryl or heteroaryl; and wherein the disease or disorder is cardiometabolic syndrome, a diabetic kidney disease, a lipid disorder, muscle inflammation, sarcopenia or bone density loss, non-alcoholic fatty liver disease (NAFLD), non-alcoholic steatohepatitis (NASH), metabolic dysfunction-associated steatohepatitis (MASH), hypercholesterolemia, hypertriglyceridemia, familial cholesterolemia, familial triglyceridemia, or a cardiovascular disorder.

2. The method of embodiment 1, wherein the compound is of Formula (A-1):

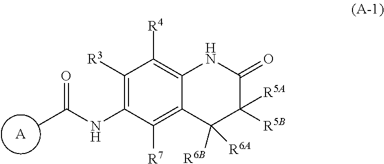

or a pharmaceutically acceptable salt, stereoisomer, tautomer, or deuterated form thereof,
wherein
ring A is heteroaryl or aryl, wherein the heteroaryl or aryl is optionally substituted with one or more substituents independently selected from the group consisting of halo, —NR⁸R⁹, —CN, —C₁₋₆ alkyl, —C₁₋₆ haloalkyl, —C₁₋₆ alkylene-carbocyclyl, or —C₁₋₆ alkylene-heterocyclyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo; and
R^{5A}, R^{5B}, R^{6A}, and R^{6B} are each independently —H, halo, or —C₁₋₆ alkyl; or one of RSA and R^{5B} is joined with one of R^{6A} and R^{6B} to form an optionally substituted C₃₋₈ carbocyclic ring, or an optionally substituted 3-8 membered heterocyclic ring.

3. The method of embodiment 2, wherein the compound is of Formula (I):

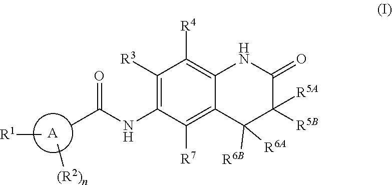

or a pharmaceutically acceptable salt, stereoisomer, tautomer, or deuterated form thereof
wherein:
ring A is heteroaryl or aryl;
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl; and
n is 1 or 2.

4. The method of embodiment 3, wherein the compound is of Formula (I-A):

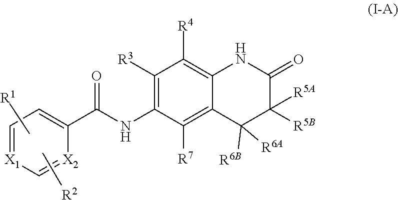

or a pharmaceutically acceptable salt, stereoisomer, tautomer, or deuterated form thereof;
wherein X¹ and X² are each independently CH or N.

5. The method of embodiment 4, wherein the compound is of Formula (I-B):

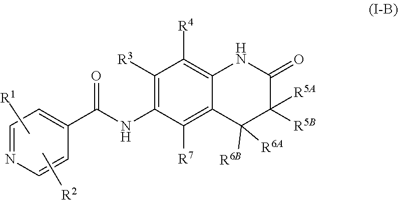

or a pharmaceutically acceptable salt, stereoisomer, tautomer, or deuterated form thereof.

6. The method of any one of embodiments 3-5, wherein R¹ is —C₁₋₆ haloalkyl.

7. The method of any one of embodiments 3-5, wherein R¹ is —C₁₋₃ haloalkyl, halo, or —NR⁸R⁹.

8. The method of embodiment 7, wherein R¹ is —C₁₋₃ haloalkyl.

9. The method of embodiment 7, wherein R¹ is —CF₃, —CH₂CF₃, Cl, F, or —NH₂.

10. The method of embodiment 7, wherein R¹ is —CF₃ or —CH₂CF₃.

11. The method of any one of embodiments 3-10, wherein R² is —CN or —C₁₋₆ alkyl.

12. The method of embodiment 11, wherein R² is —CN or —C₁₋₃ alkyl.

13. The method of embodiment 12, wherein R² is —CN, —CH₃, or —CH₂CH₃.

14. The method of any one of embodiments 3-13, wherein R³ is —H or halo.

15. The method of embodiment 14, wherein R³ is —H.

16. The method of any one of embodiments 3-15, wherein R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —NR⁸C(O)C₁₋₆ alkyl, —NR⁸R⁹, —C(O)OH, —C(O)OC₁₋₆ alkyl, heterocyclyl, heteroaryl, or carbocyclyl.

17. The method of embodiment 16, wherein R⁴ is halo, —CN, —C₁₋₆alkyl, —C₁₋₆ alkyl-CN, —C₁₋₆ haloalkyl, or carbocyclyl.

18. The method of embodiment 17, wherein R⁴ is halo or —C₁₋₆alkyl.

19. The method of embodiment 18, wherein R⁴ is —F or —CH₃.

20. The method of embodiment 19, wherein R⁴ is —CH₃.

21. The method of any one of embodiments 3-20, wherein R^{5A}, R^{5B}, R^{6A}, and R6B are —H.

22. The method of any one of embodiments 3-21, wherein R⁷ is —H or —C₁₋₆alkyl.

23. The method of any one of embodiments 3-22, wherein R⁸ is —H or —C₁₋₆alkyl.

24. The method of embodiment 1, wherein the compound is selected from the group consisting of:

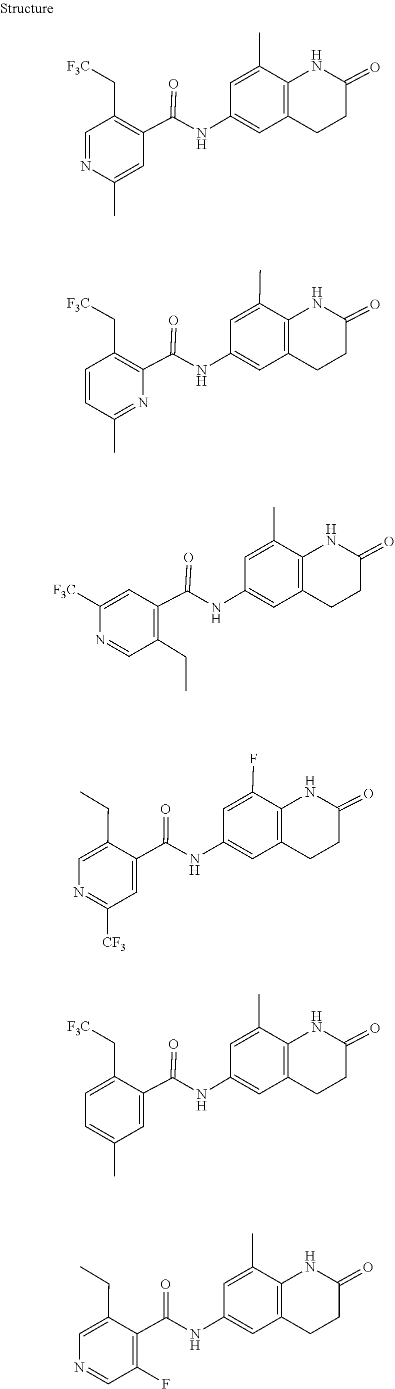 [SMILES: Cc1cc(C(=O)Nc2cc(C)c3c(c2)CCC(=O)N3)c(CC(F)(F)F)cn1 ; Cc1ccc(CC(F)(F)F)c(C(=O)Nc2cc(C)c3c(c2)CCC(=O)N3)n1 ; CCc1cnc(C(F)(F)F)cc1C(=O)Nc1cc(C)c2c(c1)CCC(=O)N2 ; CCc1cnc(C(F)(F)F)cc1C(=O)Nc1cc(F)c2c(c1)CCC(=O)N2 ; Cc1ccc(CC(F)(F)F)c(C(=O)Nc2cc(C)c3c(c2)CCC(=O)N3)c1 ; CCc1cncc(F)c1C(=O)Nc1cc(C)c2c(c1)CCC(=O)N2]

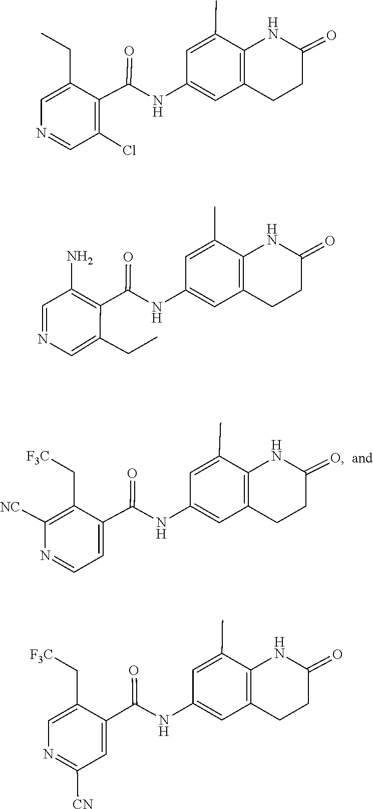

or a pharmaceutically acceptable salt, stereoisomer, tautomer, or deuterated form thereof.

25. The method of embodiment 1 or 2, wherein the compound is of Formula (II):

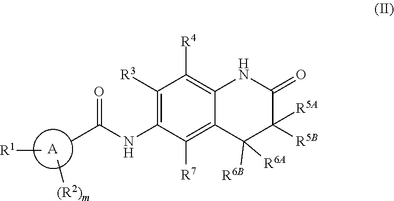

or a pharmaceutically acceptable salt, stereoisomer, tautomer, or deuterated form thereof;
wherein:
ring A is heteroaryl or aryl;
R¹ is —C₁₋₆ haloalkyl, halo or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
m is 0, 1 or 2;
R³ is —H, —C₁₋₆ alkyl, or —C₁₋₆ haloalkyl; and
R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl.

26. The method of embodiment 25, wherein ring A is heteroaryl.

27. The method of embodiment 25 or 26, wherein ring A is a 6-membered heteroaryl.

28. The method of any one of embodiments 25-27, wherein the compound of Formula (II) is a compound of Formula (II-A):

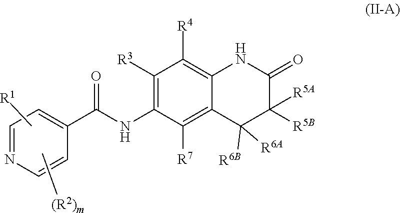

or a pharmaceutically acceptable salt, stereoisomer, tautomer, or deuterated form thereof.

29. The method of any one of embodiments 25-28, wherein the compound of Formula (II-A) is a compound of Formula (II-B):

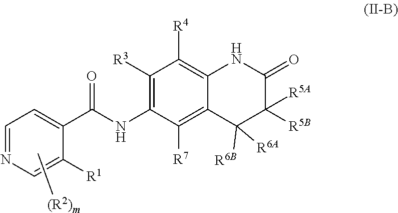

or a pharmaceutically acceptable salt, stereoisomer, tautomer, or deuterated form thereof.

30. The method of any one of embodiments 25-29, wherein R¹ is —C₁₋₃ haloalkyl.

31. The method of embodiment 30, wherein R¹ is —CH₂CF₃.

32. The method of any one of embodiments 25-31, wherein m is 0.

33. The method of any one of embodiments 25-32, wherein R³ is —H.

34. The method of any one of embodiments 25-33, wherein R⁴ is —F, —CN, —OH, —C₁₋₆ alkyl-CN, —OC₁₋₆ alkyl, —C(O)NR⁸R⁹, —NR⁸C(O)C₁₋₆ alkyl, —NR⁸R⁹, —C(O)OH, —C(O)OC₁₋₆ alkyl, heterocyclyl, or heteroaryl.

35. The method of any one of embodiments 25-34, wherein R⁴ is —F, —CN, OC₁₋₆ alkyl, or —C(O)N(R⁸R⁹)₂.

36. The method of any one of embodiments 25-34, wherein R⁴ is a 5-6 membered heteroaryl.

37. The method of embodiment 36, wherein the heteroaryl is a 5-membered heteroaryl containing two nitrogen atoms.

38. The method of any one of embodiments 25-34, wherein R⁴ is —F, —CN, —OCH₃, —C(O)NH₂,

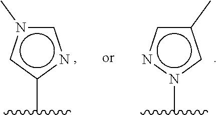

39. The method of any one of embodiments 25-38, wherein R^{5A}, R^{5B}, R^{6A}, and R^{6B} are —H.

40. The method of any one of embodiments 25-39, wherein R⁷ is —H or —C₁₋₆alkyl.

41. The method of any one of embodiments 25-40, wherein R⁸ is —H or —C₁₋₆alkyl.

42. The method of embodiment 1, wherein the compound is selected from the group consisting of:

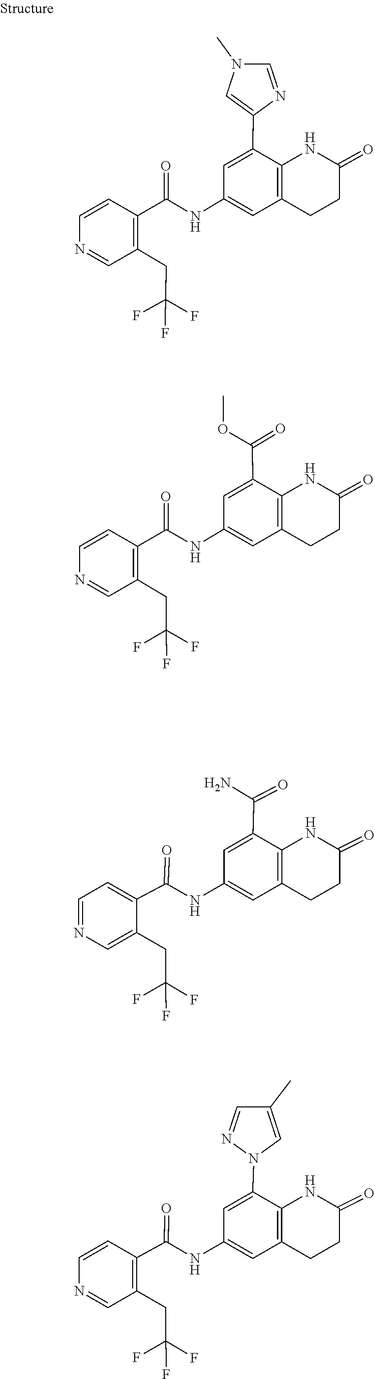 [SMILES: Cn1cnc(-c2cc(NC(=O)c3ccncc3CC(F)(F)F)cc3c2NC(=O)CC3)c1 ; COC(=O)c1cc(NC(=O)c2ccncc2CC(F)(F)F)cc2c1NC(=O)CC2 ; NC(=O)c1cc(NC(=O)c2ccncc2CC(F)(F)F)cc2c1NC(=O)CC2 ; Cc1cnn(-c2cc(NC(=O)c3ccncc3CC(F)(F)F)cc3c2NC(=O)CC3)c1]

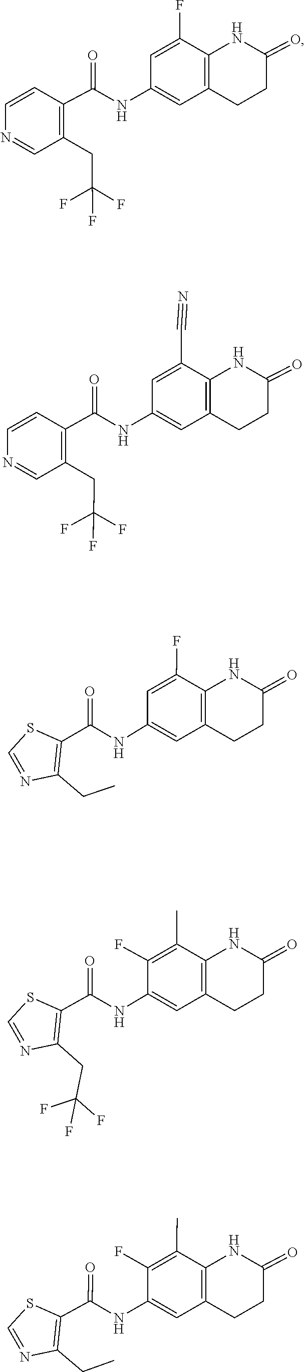 [SMILES: O=C1CCc2cc(NC(=O)c3ccncc3CC(F)(F)F)cc(F)c2N1 ; N#Cc1cc(NC(=O)c2ccncc2CC(F)(F)F)cc2c1NC(=O)CC2 ; CCc1ncsc1C(=O)Nc1cc(F)c2c(c1)CCC(=O)N2 ; Cc1c(F)c(NC(=O)c2scnc2CC(F)(F)F)cc2c1NC(=O)CC2 ; CCc1ncsc1C(=O)Nc1cc2c(c(C)c1F)NC(=O)CC2]

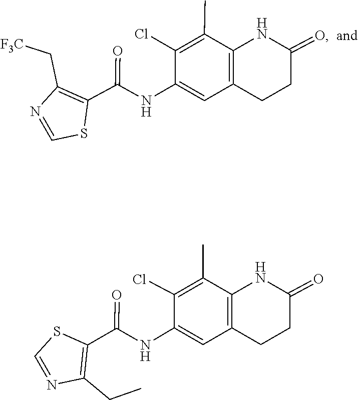

or a pharmaceutically acceptable salt, stereoisomer, tautomer, or deuterated form thereof.

43. The method of embodiment 1, wherein a compound of Formula (A) or a pharmaceutically acceptable salt, stereoisomer, tautomer, or deuterated form thereof is administered.

44. The method of embodiment 1, wherein a pharmaceutical composition comprising a compound of Formula (A) or a pharmaceutically acceptable salt, stereoisomer, tautomer, or deuterated form thereof, and a pharmaceutically acceptable carrier is administered.

45. The method of any one of embodiments 1-44, wherein the lipid disorder is hyperlipidemia or dyslipidemia.

46. The method of any one of embodiments 1-44 wherein the cardiovascular disorder is pulmonary arterial hypertension, chronic heart failure, neointimal hyperplasia, Atherosclerotic Cardiovascular Disease (ASCVD), heart failure with preserved ejection fraction, ischemic heart disease or coronary artery disease.

### NUMBERED EMBODIMENTS #2

1. A compound of Formula (III):

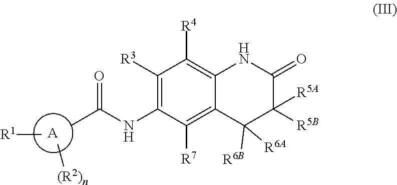

or a pharmaceutically acceptable salt, stereoisomer, tautomer, or deuterated form thereof;
wherein:
ring A is heteroaryl or aryl;
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
n is 1 or 2;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo;
R^{5A}, R^{5B}, R^{6A}, and R^{6B} are each independently —H, halo, or —C₁₋₆ alkyl; or
one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl; and provided the compound is not:
5-Isothiazolecarboxamide, 3-methyl-N-(1,2,3,4-tetrahydro-8-methyl-2-oxo-6-quinolinyl)-4-(2,2,2-trifluoroethyl)-;
5-Isothiazolecarboxamide, 3-cyano-N-(1,2,3,4-tetrahydro-8-methyl-2-oxo-6-quinolinyl)-4-(2,2,2-trifluoroethyl)-;
4-Pyridinecarboxamide, 2-chloro-5-ethyl-N-(1,2,3,4-tetrahydro-8-methyl-2-oxo-6-quinolinyl)-;
5H-Cyclopenta[b]pyridine-4-carboxamide, 6,6-difluoro-6,7-dihydro-N-(1,2,3,4-tetrahydro-8-methyl-2-oxo-6-quinolinyl)-; or
4-Quinolinecarboxamide, 6,6-difluoro-5,6,7,8-tetrahydro-N-(1,2,3,4-tetrahydro-8-methyl-2-oxo-6-quinolinyl)-.

2. The compound of embodiment 1, wherein the compound is of Formula (III-A):

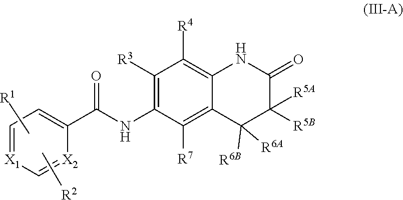

or a pharmaceutically acceptable salt, stereoisomer, tautomer, or deuterated form thereof;
wherein X¹ and X² are each independently CH or N.
3. The compound of embodiment 2, wherein the compound is of Formula (III-B):

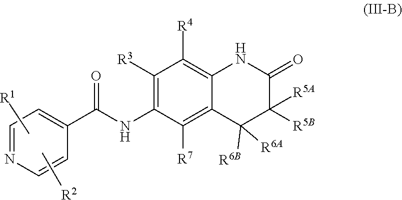

or a pharmaceutically acceptable salt, stereoisomer, tautomer, or deuterated form thereof.

4. The compound of any one of embodiments 1-3, wherein R¹ is —C₁₋₆ haloalkyl.

5. The compound of any one of embodiments 1-3, wherein R¹ is —C₁₋₃ haloalkyl, halo, or —NR⁸R⁹.

6. The compound of embodiment 5, wherein R¹ is —C₁₋₃ haloalkyl.

7. The compound of embodiment 5, wherein R¹ is —CF₃, —CH₂CF₃, Cl, F, or —NH₂.

8. The compound of embodiment 5, wherein R¹ is —CF₃ or —CH₂CF₃.

9. The compound of any one of embodiments 1-8, wherein R² is —CN or —C₁₋₆ alkyl.

10. The compound of embodiment 9, wherein R² is —CN or —C₁₋₃ alkyl.

11. The compound of embodiment 10, wherein R² is —CN, —CH₃, or —CH₂CH₃.

12. The compound of any one of embodiments 1-11, wherein R³ is —H or halo.

13. The compound of embodiment 12, wherein R³ is —H.

14. The compound of any one of embodiments 1-13, wherein R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —NR⁸C(O)C₁₋₆ alkyl, —NR⁸R⁹, —C(O)OH, —C(O)OC₁₋₆ alkyl, heterocyclyl, heteroaryl, or carbocyclyl.

15. The compound of embodiment 14, wherein R⁴ is halo, —CN, —C₁₋₆alkyl, —C₁₋₆ alkyl-CN, —C₁₋₆ haloalkyl, or carbocyclyl.

16. The compound of embodiment 15, wherein R⁴ is halo or —C₁₋₆alkyl.

17. The compound of embodiment 16, wherein R⁴ is —F or —CH₃.

18. The compound of embodiment 17, wherein R⁴ is —CH₃.

19. The compound of any one of embodiments 1-18, wherein R^{5A}, RSB, R^{6A}, and R^{6B} are—H.

20. The compound of any one of embodiments 1-19, wherein R⁷ is —H or —C₁₋₆alkyl.

21. The compound of any one of embodiments 1-20, wherein R⁸ is —H or —C₁₋₆alkyl.

22. The compound of embodiment 1 selected from the group consisting of:

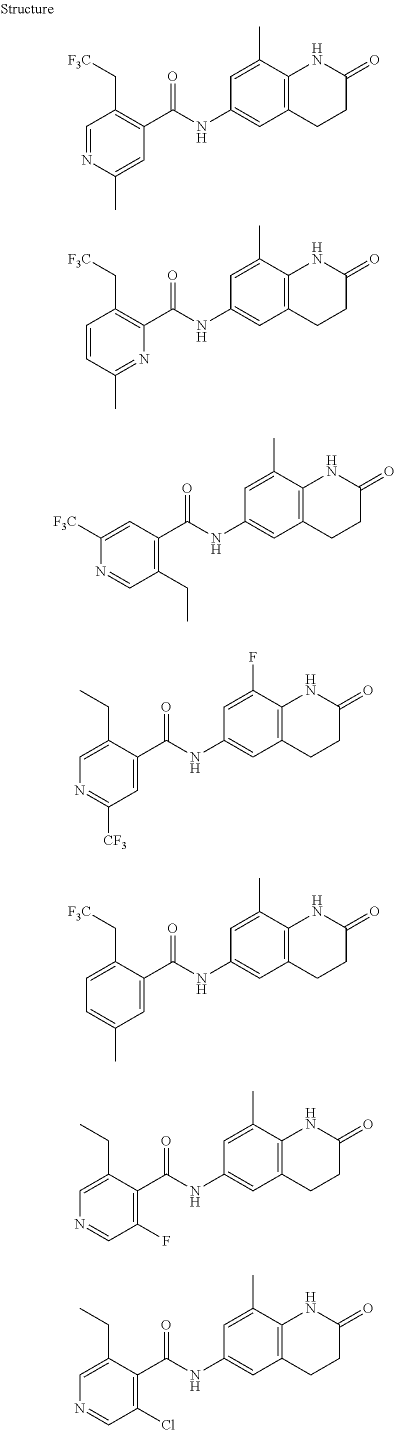

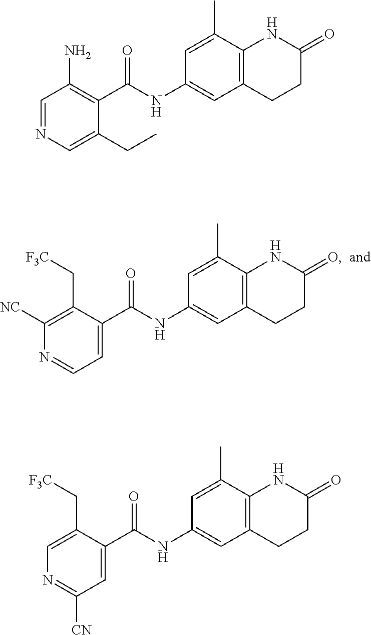

or a pharmaceutically acceptable salt, stereoisomer, tautomer, or deuterated form thereof.

23. A compound of Formula (IV):

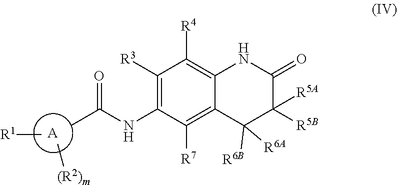

or a pharmaceutically acceptable salt, stereoisomer, tautomer, or deuterated form thereof;
wherein:
ring A is heteroaryl or aryl;
R¹ is —C₁₋₆ haloalkyl, halo or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
m is 0, 1 or 2;
R³ is —H, —C₁₋₆ alkyl, or —C₁₋₆ haloalkyl;
R⁴ is —F, —CN, —OH, —O—C₁₋₆alkyl, —C₁₋₆ alkyl-CN, —C(O)NR⁸R⁹, —C(O)OR⁸, —NR⁸R⁹, —NR⁸C(O)R⁹, heterocyclyl, or heteroaryl;
R^{5A}, R^{5B}, R^{6A}, and R^{6B} are each independently —H, halo, or —C₁₋₆ alkyl; or
one of RSA and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆alkyl, aryl, or heteroaryl provided the compound is not:
5-Isothiazolecarboxamide, 3-methyl-N-(1,2,3,4-tetrahydro-8-methyl-2-oxo-6-quinolinyl)-4-(2,2,2-trifluoroethyl)-;
5-Isothiazolecarboxamide, 3-cyano-N-(1,2,3,4-tetrahydro-8-methyl-2-oxo-6-quinolinyl)-4-(2,2,2-trifluoroethyl)-;
4-Pyridinecarboxamide, 2-chloro-5-ethyl-N-(1,2,3,4-tetrahydro-8-methyl-2-oxo-6-quinolinyl)-;
5H-Cyclopenta[b]pyridine-4-carboxamide, 6,6-difluoro-6,7-dihydro-N-(1,2,3,4-tetrahydro-8-methyl-2-oxo-6-quinolinyl)-; or 4-Quinolinecarboxamide, 6,6-difluoro-5,6,7,8-tetrahydro-N-(1,2,3,4-tetrahydro-8-methyl-2-oxo-6-quinolinyl)-.

24. The compound of embodiment 23, wherein ring A is heteroaryl.

25. The compound of embodiment 23 or 24, wherein ring A is a 6-membered heteroaryl.

26. The compound of any one of embodiments 23-25, wherein the compound of Formula (IV) is a compound of Formula (IV-A):

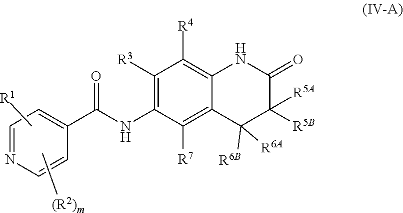

or a pharmaceutically acceptable salt, stereoisomer, tautomer, or deuterated form thereof.

27. The compound of any one of embodiments 23-26, wherein the compound of Formula (IV-A) is a compound of Formula (IV-B):

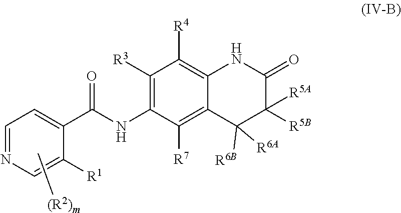

or a pharmaceutically acceptable salt, stereoisomer, tautomer, or deuterated form thereof.

28. The compound of any one of embodiments 23-27, wherein R¹ is —C₁₋₃ haloalkyl.

29. The compound of embodiment 28, wherein R¹ is —CH₂CF₃.

30. The compound of any one of embodiments 23-29, wherein m is 0.

31. The compound of any one of embodiments 23-30, wherein R³ is —H.

32. The compound of any one of embodiments 23-31, wherein R⁴ is —F, —CN, —OH, —C₁₋₆ alkyl-CN, —OC₁₋₆ alkyl, —C(O)NR⁸R⁹, —NR⁸C(O)C₁₋₆ alkyl, —NR⁸R⁹, —C(O)OH, —C(O)OC₁₋₆ alkyl, heterocyclyl, or heteroaryl.

33. The compound of any one of embodiments 23-32, wherein R⁴ is —F, —CN, OC₁₋₆ alkyl, or —C(O)N(R⁸R⁹) 2.

34. The compound of any one of embodiments 23-32, wherein R⁴ is a 5-6 membered heteroaryl.

35. The compound of embodiment 34, wherein the heteroaryl is a 5-membered heteroaryl containing two nitrogen atoms.

36. The compound of any one of embodiments 23-32, wherein R⁴ is —F, —CN, —OCH₃, —C(O)NH₂,

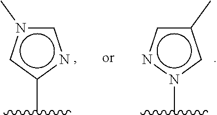

37. The compound of any one of embodiments 23-36, wherein R^{5A}, R^{5B}, R^{6A}, and R^{6B} are —H.

38. The compound of any one of embodiments 23-37, wherein R⁷ is —H or —C₁₋₆alkyl.

39. The compound of any one of embodiments 23-38, wherein R⁸ is —H or —C₁₋₆alkyl.

40. A compound is selected from the group consisting of:

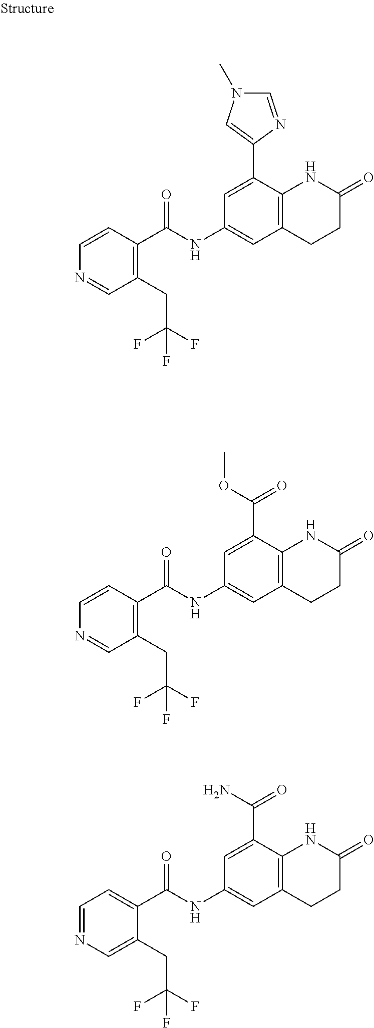 [SMILES: Cn1cnc(-c2cc(NC(=O)c3ccncc3CC(F)(F)F)cc3c2NC(=O)CC3)c1 ; COC(=O)c1cc(NC(=O)c2ccncc2CC(F)(F)F)cc2c1NC(=O)CC2 ; NC(=O)c1cc(NC(=O)c2ccncc2CC(F)(F)F)cc2c1NC(=O)CC2]

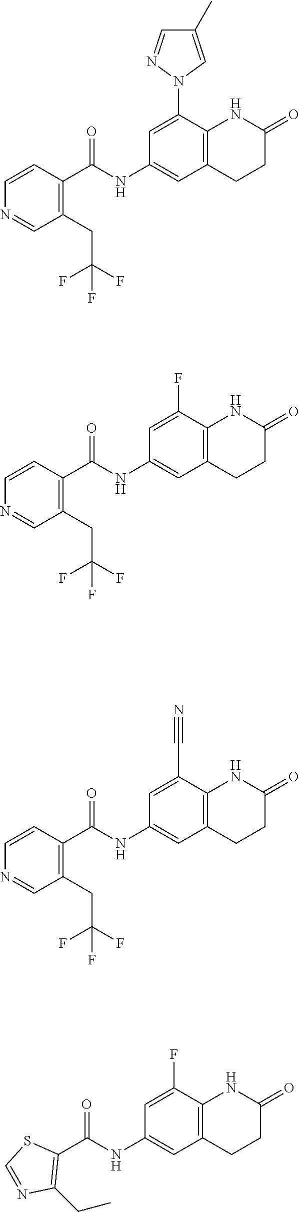 [SMILES: Cc1cnn(-c2cc(NC(=O)c3ccncc3CC(F)(F)F)cc3c2NC(=O)CC3)c1 ; O=C1CCc2cc(NC(=O)c3ccncc3CC(F)(F)F)cc(F)c2N1 ; N#Cc1cc(NC(=O)c2ccncc2CC(F)(F)F)cc2c1NC(=O)CC2 ; CCc1ncsc1C(=O)Nc1cc(F)c2c(c1)CCC(=O)N2]

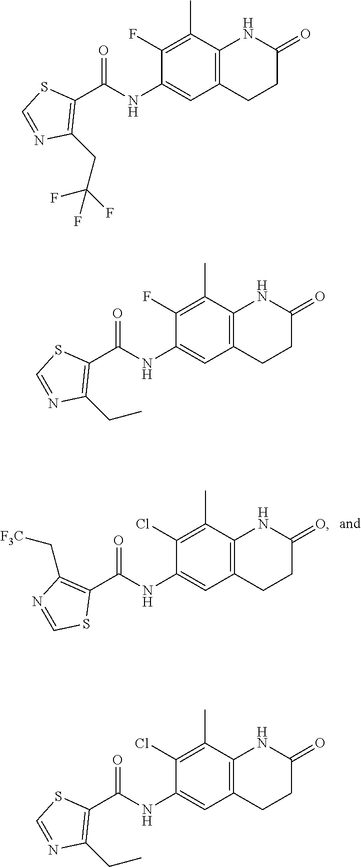 [SMILES: Cc1c(F)c(NC(=O)c2scnc2CC(F)(F)F)cc2c1NC(=O)CC2 ; CCc1ncsc1C(=O)Nc1cc2c(c(C)c1F)NC(=O)CC2 ; Cc1c(Cl)c(NC(=O)c2scnc2CC(F)(F)F)cc2c1NC(=O)CC2 ; CCc1ncsc1C(=O)Nc1cc2c(c(C)c1Cl)NC(=O)CC2]

or a pharmaceutically acceptable salt, stereoisomer, tautomer, or deuterated form thereof.

41. A pharmaceutical composition comprising the compound of any one of embodiments 1-40, or a pharmaceutically acceptable salt, stereoisomer, tautomer, or deuterated form thereof, and a pharmaceutically acceptable carrier.

42. A method for treating type 2 diabetes in a subject in need thereof, the method comprising administering to the subject an effective amount of the compound of any one of embodiments 1-40 or a pharmaceutically acceptable salt thereof, or the pharmaceutical composition of embodiment 41.

43. The method of embodiment 42, wherein treating type 2 diabetes comprises treating type 2 diabetes-associated dyslipidemia.

44. A method of treating a disease or disorder associated with aldehyde dehydrogenase in a subject in need thereof, comprising administering to the subject an effective amount of the compound of any one of embodiments 1-40, or a pharmaceutically acceptable salt thereof, or the pharmaceutical composition of embodiment 41.

45. The method of embodiment 44, wherein the disease or disorder is associated with aldehyde dehydrogenase isoform 1a3 (ALDH1a3).

46. A method of inhibiting an aldehyde dehydrogenase in a subject in need thereof, comprising administering to the subject an effective amount of the compound of any one of embodiments 1-40 or a pharmaceutically acceptable salt thereof, or the pharmaceutical composition of embodiment 41.

47. The method of embodiment 46, wherein aldehyde dehydrogenase isoform 1a3 (ALDH1a3) is selectively inhibited.

### EXAMPLES

### Example 1: Synthesis of 2-methyl-N-(8-methyl-2-oxo-1,2,3,4-tetrahydroquinolin-6-yl)-5-(2,2,2-trifluoroethyl)isonicotinamide (Compound 1)

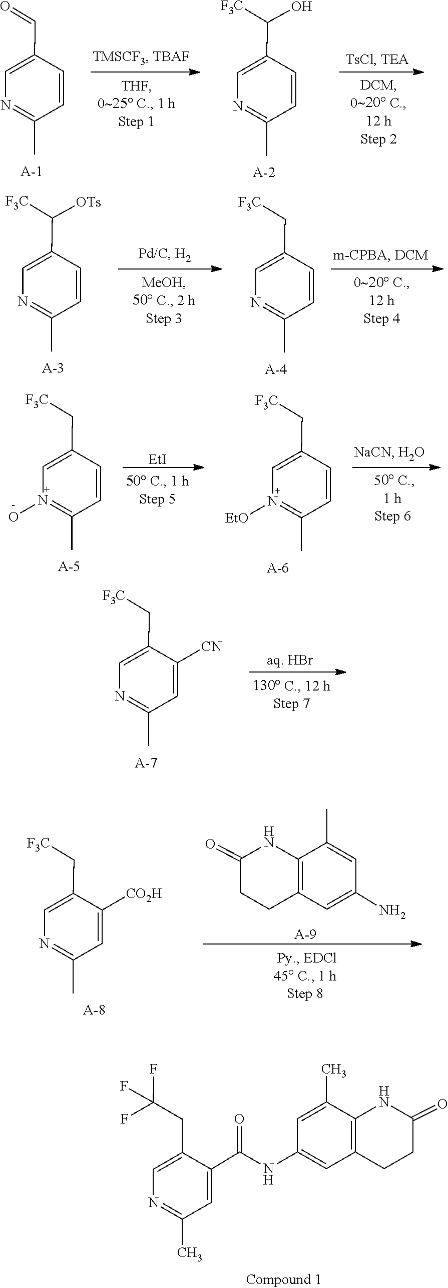

### Step 1

To a solution of 6-methylpyridine-3-carbaldehyde (5 g, 41.28 mmol, 1 eq) and TBAF (1 M, 41.28 mL, 1 eq) in THF (90 mL) was added TMSCF₃ (6.46 g, 45.40 mmol, 1.1 eq) at 0° C. The mixture was stirred at 25° C. for 1 hr. TLC indicated 6-methylpyridine-3-carbaldehyde was consumed completely and one new spot formed. To the mixture was added water (50 ml). The reaction liquid was extracted by EtOAc (100 ml), then the organic liquid was washed with brine (10 ml*3), dried over anhydrous Na₂SO₄, filtered and concentrated under reduced pressure to give a residue. The residue was purified by column chromatography (SiO₂, Petroleum ether/Ethyl acetate=15/1 to 5/1). Compound 2,2,2-trifluoro-1-(6-methyl-3-pyridyl) ethanol (3.43 g, 17.94 mmol, 21.74% yield) was obtained. ¹H NMR (400 MHz, CHLOROFORM-d) δ ppm 7.45-7.56 (m, 2H), 7.25-7.31 (m, 1H), 7.05 (d, J=5.6 Hz, 1H), 7.34-7.42 (m, 1H), 2.34 (s, 1H).

### Step 2

A mixture of 2,2,2-trifluoro-1-(6-methyl-3-pyridyl) ethanol (5 g, 26.16 mmol, 1 eq), TEA (7.94 g, 78.47 mmol, 10.92 mL, 3 eq) in DCM (100 mL) was degassed and purged with N₂ for 3 times at 0° C. To the mixture was added 4-methylbenzenesulfonyl chloride (6.48 g, 34.00 mmol, 1.3 eq) in DCM (15 mL) at 0° C. Then the mixture was stirred at 20° C. for 12 hr under N₂ atmosphere. LC-MS showed 2,2,2-trifluoro-1-(6-methyl-3-pyridyl) ethanol was consumed completely and one main peak with desired m/z was detected. To the mixture was added water (50 mL), the reaction liquid was washed by DCM (100 ml), then the organic liquid was washed with brine (5 mL*3), dried over Na₂SO₄, filtered and concentrated under reduced pressure to give a residue. The residue was purified by column chromatography (SiO₂, Petroleum ether/Ethyl acetate=10/1 to 4/1). Compound [2,2,2-trifluoro-1-(6-methyl-3-pyridyl)ethyl]4-methylbenzenesulfonate (4.8 g, 13.48 mmol, 51.54% yield) was obtained.

### Step 3

To the bottle was added Pd/C (0.5 g, 10% purity) under Ar. Then to the mixture was added [2,2,2-trifluoro-1-(6-methyl-3-pyridyl)ethyl]4-methylbenzenesulfonate (3 g, 8.69 mmol, 1 eq) in MeOH (100 mL). The suspension was degassed under vacuum and purged with H₂ 3 times. The mixture was stirred under H₂ (50 psi) at 50° C. for 2 hours. LC-MS showed [2,2,2-trifluoro-1-(6-methyl-3-pyridyl)ethyl]4-methylbenzenesulfonate was consumed completely and one main peak with desired m/z was detected. The reaction mixture was filtered and the filtrate was concentrated. To the mixture was added sat. NaHCO₃ (50 mL). The aqueous phase was extracted with DCM (20 mL*3). The combined organic phase was washed with brine (30 mL), dried with Na₂SO₄, filtered and concentrated in vacuum. 2-methyl-5-(2,2,2-trifluoroethyl)pyridine (1.5 g, 8.56 mmol, 98.58% yield) was obtained.

### Step 4

To the solution of 2-methyl-5-(2,2,2-trifluoroethyl)pyridine (1.5 g, 8.56 mmol, 1 eq) in DCM (30 mL) was added m-CPBA (2.09 g, 10.28 mmol, 85% purity, 1.2 eq) in portions at 0° C. The mixture was stirred at 20° C. for 12 hr. LC-MS showed 2-methyl-5-(2,2,2-trifluoroethyl)pyridine was consumed completely and one main peak with desired m/z was detected. To the mixture was added to sat. Na₂SO₃ (50 mL). The mixture was stirred at 20° C. for 0.5 hr. The aqueous phase was extracted with DCM (50 mL*3). The combined organic phase was washed with brine (20 mL), dried with Na₂SO₄, filtered and concentrated in vacuum. The residue was purified by flash silica gel chromatography (ISCO®; 20 g SepaFlash® Silica Flash Column, Eluent of 0~100% Ethyl acetate/Petroleum ethergradient @ 80 mL/min). 2-methyl-1-oxido-5-(2,2,2-trifluoroethyl)pyridin-1-ium (1 g, 5.23 mmol, 61.09% yield) was obtained. ¹H NMR (400 MHz, CHLOROFORM-d) δ ppm 8.25 (s, 1H), 7.26 (d, J=8.0 Hz, 1H), 7.13 (d, J=8.0 Hz, 1H), 3.25-3.40 (m, 2H), 2.52 (s, 3H).

### Step 5

The mixture of 2-methyl-1-oxido-5-(2,2,2-trifluoroethyl)pyridin-1-ium (1 g, 5.23 mmol, 1 eq) in EtI (8.16 g, 52.31 mmol, 4.18 mL, 10 eq) was stirred at 50° C. for 1 hr. LC-MS showed ~30.8% of 2-methyl-1-oxido-5-(2,2,2-trifluoroethyl)pyridin-1-ium remained. Several new peaks were shown on LC-MS and ~28.9% of desired compound was detected. The mixture was cooled to 20° C. To the mixture was added Petroleum ether (20 mL). The mixture was stirred at 20° C. for 20 min. The mixture was filtered. The filter cake was concentrated in vacuum. 1-ethoxy-2-methyl-5-(2,2,2-trifluoroethyl)pyridin-1-ium (1.1 g, 3.17 mmol, 60.58% yield) was obtained.

### Step 6

To a 100 mL three neck bottle was added 1-ethoxy-2-methyl-5-(2,2,2-trifluoroethyl)pyridin-1-ium (1.1 g, 3.17 mmol, 1 eq, I—) in H₂O (20 mL). Then to the mixture was added the solution of NaCN (200 mg, 4.08 mmol, 1.29 eq) in H₂O (2 mL) dropwise at 50° C. The mixture was stirred at 50° C. for 1 h. LC-MS showed 1-ethoxy-2-methyl-5-(2,2,2-trifluoroethyl)pyridin-1-ium was consumed completely and two peaks with desired m/z was detected. The mixture was cooled to 25° C. and adjusted the pH to 9~10 by sat. NaOH. The aqueous phase was extracted with ethyl acetate (20 mL*3). The combined organic phases were washed with brine (20 mL), dried over Na₂SO₄, filtered and concentrated in vacuum. The residue was purified by prep-TLC (SiO₂, Petroleum ether: Ethyl acetate=2:1). 2-methyl-5-(2,2,2-trifluoroethyl)pyridine-4-carbonitrile (90 mg, 449.64 μmol, 14.18% yield) was obtained. ¹H NMR (400 MHz, CHLOROFORM-d) δ ppm 8.68 (s, 1H), 7.45 (s, 1H), 3.55-3.70 (m, 2H), 2.65 (s, 3H).

### Step 7

The mixture of 2-methyl-5-(2,2,2-trifluoroethyl)pyridine-4-carbonitrile (30 mg, 149.88 μmol, 1 eq) in HBr (1.55 g, 9.21 mmol, 1.04 mL, 48% purity, 61.43 eq) was stirred at 130° C. for 12 hr. LC-MS showed 2-methyl-5-(2,2,2- trifluoroethyl)pyridine-4-carbonitrile was consumed completely and one main peak with desired m/z was detected. The mixture was concentrated in vacuum. 2-methyl-5-(2,2,2-trifluoroethyl)pyridine-4-carboxylic acid (40 mg, crude, HBr) was obtained. ¹H NMR (400 MHz, METHANOL-d₄) δ ppm 8.93 (s, 1H), 8.36 (s, 1H), 4.20-4.30 (m, 2H), 2.85 (s, 3H).

### Step 8

To the mixture of 6-amino-8-methyl-3,4-dihydro-1H-quinolin-2-one (35 mg, 198.62 μmol, 1 eq) and 2-methyl-5-(2,2,2-trifluoroethyl)pyridine-4-carboxylic acid (65.56 mg, 218.48 μmol, 1.1 eq, HBr) in Py. (1 mL) was added EDCI (57.11 mg, 297.93 μmol, 1.5 eq). The mixture was stirred at 45° C. for 1 hr. LC-MS showed 6-amino-8-methyl-3,4-dihydro-1H-quinolin-2-one was consumed completely and one main peak with desired m/z was detected. The mixture was concentrated in vacuum. Combined with another batch (18.84 mg). The combined crude product was purified by prep-TLC (SiO₂, Petroleum ether:Ethyl acetate=0:1). 2-methyl-N-(8-methyl-2-oxo-3,4-dihydro-1H-quinolin-6-yl)-5-(2,2,2-trifluoroethyl)pyridine-4-carboxamide (30 mg, 31.1% yield) was obtained. (M+H)⁺: 378.1. ¹H NMR (400 MHz, METHANOL-d₄) δ ppm 8.53 (s, 1H), 7.52 (s, 1H), 7.42 (s, 1H), 7.35 (s, 1H), 3.80-3.95 (m, 2H), 2.98 (t, J=8.0 Hz, 2H), 2.55-2.65 (m, 5H), 2.3 (s, 3H).

### Example 2: Synthesis of 6-methyl-N-(8-methyl-2-oxo-1,2,3,4-tetrahydroquinolin-6-yl)-3-(2,2,2-trifluoroethyl)picolinamide (Compound 2)

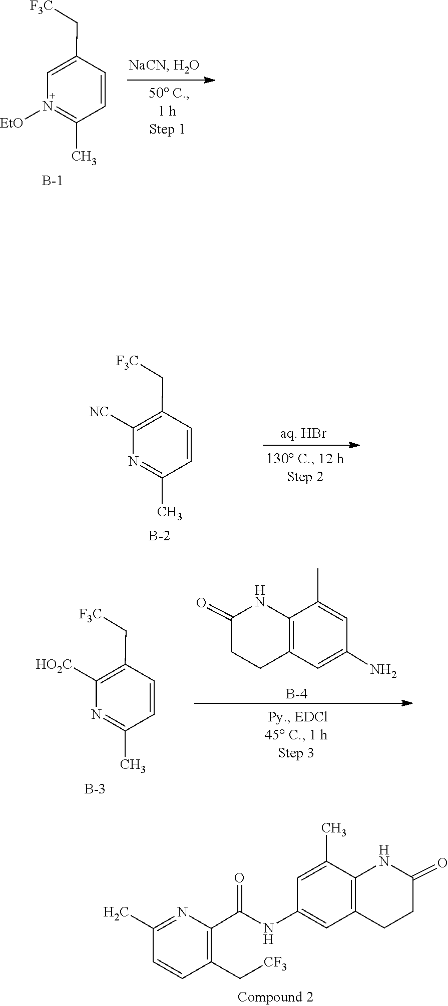

### Step 1

To a 100 mL three neck bottle was added 1-ethoxy-2-methyl-5-(2,2,2-trifluoroethyl)pyridin-1-ium (1.1 g, 3.17 mmol, 1 eq, I—) in H₂O (20 mL). Then to the mixture was added the solution of NaCN (200 mg, 4.08 mmol, 1.29 eq) in H₂O (2 mL) dropwise at 50° C. The mixture was stirred at 50° C. for 1 h. LC-MS showed 1-ethoxy-2-methyl-5-(2,2,2-trifluoroethyl)pyridin-1-ium was consumed completely and two peaks with desired m/z was detected. The mixture was cooled to 25° C. and adjusted to pH=9~10 by sat. NaOH. The aqueous phase was extracted with ethyl acetate (20 mL*3). The combined organic phase was washed with brine (20 mL), dried with anhydrous Na₂SO₄, filtered and concentrated in vacuum. The residue was purified by prep-TLC (SiO₂, Petroleum ether:Ethyl acetate=2:1). 6-methyl-3-(2,2,2-trifluoroethyl)pyridine-2-carbonitrile (30 mg, 149.88 μmol, 4.73% yield) was obtained. ¹H NMR (400 MHz, CHLOROFORM-d) δ ppm 7.73 (d, J=12.0 Hz, 1H), 7.41 (d, J=12.0 Hz, 1H), 3.60-3.70 (m, 2H), 2.63 (s, 3H).

### Step 2

The mixture of 6-methyl-3-(2,2,2-trifluoroethyl)pyridine-2-carbonitrile (30.00 mg, 149.88 μmol, 1 eq) in HBr (745.00 mg, 4.42 mmol, 500.00 μL, 48% purity, 29.49 eq) was stirred at 130° C. for 12 hr. LC-MS showed 6-methyl-3-(2,2,2-trifluoroethyl)pyridine-2-carbonitrile was consumed completely and one main peak with desired m/z was detected. The mixture was concentrated in vacuum. 6-methyl-3-(2,2,2-trifluoroethyl)pyridine-2-carboxylic acid (38 mg, 126.64 μmol, 84.49% yield, HBr) was obtained.

### Step 3

To the mixture of 6-amino-8-methyl-3,4-dihydro-1H-quinolin-2-one (20 mg, 113.50 μmol, 1 eq) and 6-methyl-3-(2,2,2-trifluoroethyl)pyridine-2-carboxylic acid (38 mg, 126.64 μmol, 1.12 eq, HBr) in Py (2 mL) was added EDCI (32.64 mg, 170.25 μmol, 1.5 eq). The mixture was stirred at 45° C. for 1 hr. LC-MS showed 6-amino-8-methyl-3,4-dihydro-1H-quinolin-2-one was consumed completely and one main peak with desired m/z was detected. The mixture was concentrated in vacuum. The residue was purified by prep-TLC (SiO₂, Petroleum ether:Ethyl acetate 0:1). 6-methyl-N-(8-methyl-2-oxo-3,4-dihydro-1H-quinolin-6-yl)-3-(2,2,2-trifluoroethyl)pyridine-2-carboxamide (9 mg, 23.85 μmol, 21.01% yield) was obtained. (M+H)⁺: 378.1. ¹H NMR (400 MHz, METHANOL-d₄) δ ppm 7.81 (d, J=8.0 Hz, 1H), 7.40-7.50 (m, 3H), 4.19-4.31 (m, 2H), 2.97 (t, J=8.0 Hz, 2H), 2.50-2.65 (m, 5H), 2.29 (s, 3H).

### Example 3: Synthesis of 5-ethyl-N-(8-methyl-2-oxo-1,2,3,4-tetrahydroquinolin-6-yl)-2-(trifluoromethyl)isonicotinamide (Compound 3)

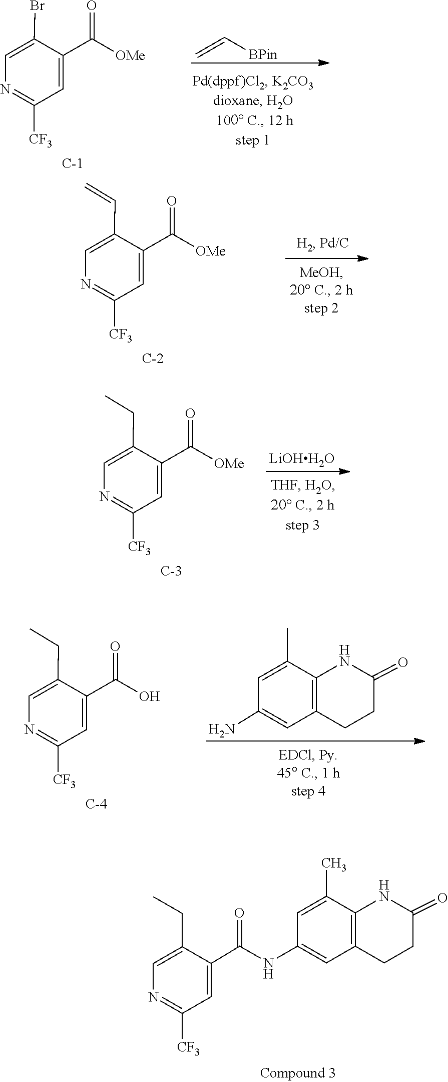

### Step 1

A mixture of methyl 5-bromo-2-(trifluoromethyl)pyridine-4-carboxylate (2 g, 7.04 mmol, 1 eq), 4,4,5,5-tetramethyl-2-vinyl-1,3,2-dioxaborolane (1.14 g, 7.39 mmol, 1.25 mL, 1.05 eq), K₂CO₃ (1.95 g, 14.08 mmol, 2 eq), Pd(dppf)Cl₂ (515.23 mg, 704.15 μmol, 0.1 eq) in dioxane (20 mL) and H₂O (2 mL) was degassed and purged with N₂ for 3 times, and then the mixture was stirred at 100° C. for 12 hr under N₂ atmosphere. TLC indicated the reaction consumed completely. The reaction mixture was diluted with H₂O 20 mL and extracted with EtOAc 40 mL (20 mL*2). The combined organic layers were dried over Na₂SO₄, filtered and concentrated under reduced pressure to give a residue. The residue was purified by column chromatography (SiO₂, Petroleum ether/Ethyl acetate=100/1 to 10/1). Methyl 2-(trifluoromethyl)-5-vinyl-pyridine-4-carboxylate (1 g, 4.33 mmol, 61% yield) was obtained. ¹H NMR (400 MHz, CHLOROFORM-d) δ ppm 8.97 (s, 1H), 8.09 (s, 1H), 7.40-7.48 (m, 1H), 5.87 (d, J=17.6 Hz, 1H), 5.62 (d, J=11.2 Hz, 1H), 3.99 (s, 3H).

### Step 2

To a solution of Pd/C (0.1 g, 2.16 mmol, 10% purity) in MeOH (5 mL) was added methyl 2-(trifluoromethyl)-5-vinyl-pyridine-4-carboxylate (0.5 g, 2.16 mmol, 1 eq) under N₂ atmosphere. The suspension was degassed and purged with H₂ for 3 times. The mixture was stirred under H₂ (15 Psi) at 20° C. for 2 hr. LCMS showed the reaction was consumed completely. The mixture was filtered and the filtrate was concentrated in vacuum. Methyl 5-ethyl-2-(trifluoromethyl)pyridine-4-carboxylate (0.5 g, crude) was obtained.

### Step 3

To a solution of methyl 5-ethyl-2-(trifluoromethyl)pyridine-4-carboxylate (400 mg, 1.72 mmol, 1 eq) in THF (4 mL) was added LiOH·H₂O (143.97 mg, 3.43 mmol, 2 eq) in H₂O (4 mL). The mixture was stirred at 20° C. for 2 hr. TLC indicated the reaction was consumed completely. The reaction mixture was diluted with H₂O 10 mL and extracted with EtOAc 10 mL (5 mL*2). The water layer was adjusted to pH=2 by addition of 1N HCl. The mixture was filtered and the filter cake concentrated in vacuum without further purification. 5-ethyl-2-(trifluoromethyl)pyridine-4-carboxylic acid (0.4 g, 85% yield) was obtained.

### Step 4

To a solution of 5-ethyl-2-(trifluoromethyl)pyridine-4-carboxylic acid (70 mg, 319.40 μmol, 1 eq) and 6-amino-8-methyl-3,4-dihydro-1H-quinolin-2-one (61.91 mg, 351.34 μmol, 1.1 eq) in Py (1 mL) was added EDCI (91.84 mg, 479.10 μmol, 1.5 eq). The mixture was stirred at 45° C. for 1 hr. TLC indicated the reaction was consumed completely. The reaction mixture was concentrated in vacuum. The residue was purified by prep-TLC (SiO₂, Ethyl acetate:MeOH=20:1). 5-ethyl-N-(8-methyl-2-oxo-3,4-dihydro-1H-quinolin-6-yl)-2-(trifluoromethyl)pyridine-4-carboxamide (52 mg, 48% yield) was obtained. (M+H)⁺: 378.1. ¹H NMR (400 MHz, DMSO-d₆) δ ppm 10.49 (s, 1H), 9.46 (s, 1H), 8.79 (s, 1H), 7.93 (s, 1H), 7.35 (d, J=29.2 Hz, 2H), 2.77-2.90 (m, 4H), 2.39-2.46 (m, 2H), 2.21 (s, 3H), 1.20 (t, J=7.6 Hz, 3H).

### Example 4: Synthesis of 5-ethyl-N-(8-fluoro-2-oxo-1,2,3,4-tetrahydroquinolin-6-yl)-2-(trifluoromethyl)isonicotinamide (Compound 4)

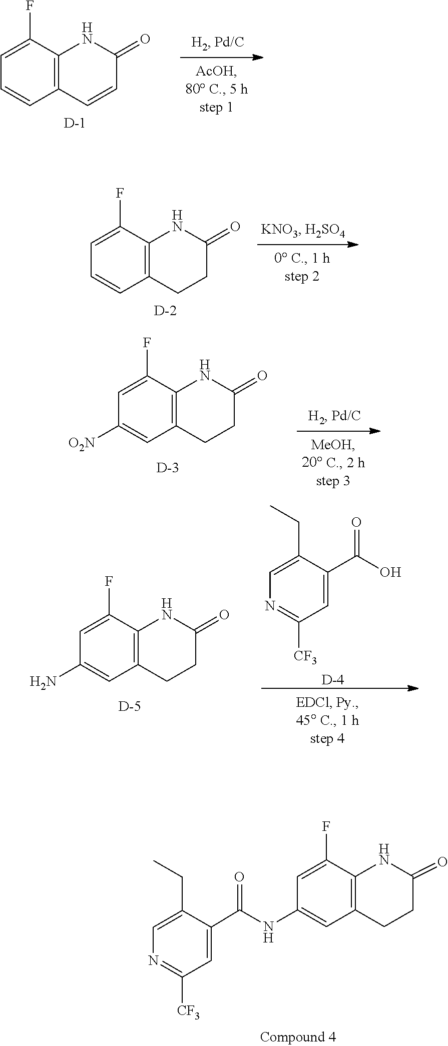

### Step 1

To a solution of Pd/C (200 mg, 6.13 mmol, 10% purity) in AcOH (20 mL) was added 8-fluoro-1H-quinolin-2-one (2 g, 6.13 mmol, 1 eq) under N₂ atmosphere. The suspension was degassed and purged with H₂ for 2 times. The mixture was stirred under H₂ (50 Psi.) at 80° C. for 5 hr. LCMS showed 8-fluoro-1H-quinolin-2-one was consumed completely. The reaction mixture was filtered and concentrated under reduced pressure to give a residue. Compound 8-fluoro-3,4-dihydro-1H-quinolin-2-one (1.4 g, crude) was obtained.

### Step 2

To a 100 mL three neck bottle was added the solution of 8-fluoro-3,4-dihydro-1H-quinolin-2-one (0.7 g, 4.24 mmol, 1 eq) in H₂SO₄ (5 mL). Then to the mixture was added KNO₃ (660 mg, 6.53 mmol, 1.54 eq) in portions at 0° C. The mixture was stirred at 0° C. for 1 hr. TLC indicated 8-fluoro-3,4-dihydro-1H-quinolin-2-one was consumed completely and two new spots formed. The mixture was poured into ice (15 mL) slowly. The mixture was filtered and the filter cake was concentrated under reduced pressure to give a residue. Compound 8-fluoro-6-nitro-3,4-dihydro-1H-quinolin-2-one (0.8 g, crude) was obtained.

### Step 3

To a solution of Pd/C (20 mg, 10% purity) in MeOH (10 mL) was added 8-fluoro-6-nitro-3,4-dihydro-1H-quinolin-2-one (0.8 g, 3.81 mmol, 1 eq) under N₂ atmosphere. The suspension was degassed and purged with H₂ for 3 times. The mixture was stirred under H₂ (15 Psi.) at 20° C. for 2 hr. TLC indicated 8-fluoro-6-nitro-3,4-dihydro-1H-quinolin-2-one was consumed completely and three new spots formed. The reaction mixture was filtered and concentrated under reduced pressure to give a residue. The residue was purified by prep-TLC (SiO₂, Petroleum ether/Ethyl acetate=0:1).

Compound 6-amino-8-fluoro-3,4-dihydro-1H-quinolin-2-one (0.5 g, 2.22 mmol, 58.32% yield) was obtained. (M+H)⁺: 181.2. ¹H NMR (400 MHz, CHLOROFORM-d) δ ppm 7.5 (br s, 1H), 6.30-6.36 (m, 2H), 3.60 (s, 2H), 2.90 (t, J=7.2 Hz, 2H), 2.61 (t, J=7.2 Hz, 2H).

### Step 4

To a solution of 6-amino-8-fluoro-3,4-dihydro-1H-quinolin-2-one (50 mg, 277.50 μmol, 1.1 eq) and 5-ethyl-2-(trifluoromethyl)pyridine-4-carboxylic acid (55.29 mg, 252.27 μmol, 1 eq) in Py (1 mL) was added EDCI (72.54 mg, 378.41 μmol, 1.5 eq). The mixture was stirred at 45° C. for 1 h. LC-MS showed 6-amino-8-fluoro-3,4-dihydro-1H-quinolin-2-one was consumed completely and one main peak with desired m/z was detected. The reaction mixture was filtered and concentrated under reduced pressure to give a residue. The residue was purified by column chromatography (SiO₂, Petroleum ether/Ethyl acetate=1/1). Compound 5-ethyl-N-(8-fluoro-2-oxo-3,4-dihydro-1H-quinolin-6-yl)-2-(trifluoromethyl)pyridine-4-carboxamide (40 mg, 99.97 μmol, 39.63% yield) was obtained. (M+H)⁺: 382.1. 1H NMR (400 MHz, METHANOL-d₄) δ ppm 8.73 (s, 1H) 7.86 (s, 1H) 7.56 (dd, J=12.0, 2.0 Hz, 1H), 7.26-7.29 (m, 1H), 3.02 (t, J=7.2 Hz, 2H) 2.92 (q, J=7.6 Hz, 2H) 2.58-2.66 (m, 2H) 1.30 (t, J=8.0 Hz, 3H).

### Example 5: Synthesis of 5-methyl-N-(8-methyl-2-oxo-1,2,3,4-tetrahydroquinolin-6-yl)-2-(2,2,2-trifluoroethyl)benzamide (Compound 5)

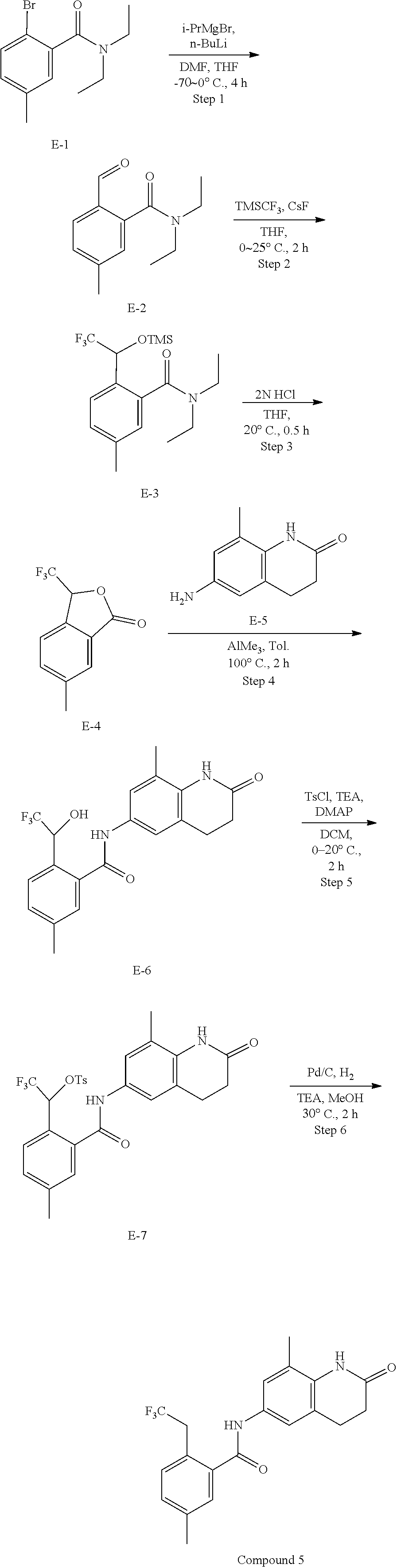

### Step 1

To the solution of i-PrMgBr (3 M, 4.44 mL, 1.2 eq) in THF (25 mL) was added n-BuLi (2.5 M, 11.10 mL, 2.5 eq) dropwise at 0° C. Then the mixture was cooled to −70° C. To the mixture was added 2-bromo-N,N-diethyl-5-methyl-benzamide (3 g, 11.10 mmol, 1 eq) in THF (15 mL) drop-wise at −70° C. The mixture was stirred at −70° C. for 1 hr. Then to the mixture was added DMF (3.25 g, 44.42 mmol, 3.42 mL, 4 eq) dropwise at −70° C. The mixture was stirred at −70° C. for 3 hr under N₂. TLC indicated ~10% of 2-bromo-N,N-diethyl-5-methyl-benzamide was remained, and one major new spot with larger polarity was detected. To the mixture was added sat. NH₄Cl (50 mL) dropwise at −20° C. The aqueous phase was extracted with ethyl acetate (30 mL*4). The combined organic phases were washed with brine (30 mL*3), dried with anhydrous Na₂SO₄, filtered and concentrated in vacuum. The residue was purified by column chromatography (SiO₂, Petroleum ether/Ethyl acetate=10/1 to 2/1). Compound N,N-diethyl-2-formyl-5-methyl-benzamide (1.2 g, 4.49 mmol, 40.41% yield) was obtained. ¹H NMR (400 MHz, CHLOROFORM-d) δ ppm 10.00 (s, 1H), 7.84 (d, J=8.0 Hz, 1H), 7.34 (d, J=8.0 Hz, 1H), 7.16 (s, 1H), 3.55-3.65 (m, 2H), 3.05-3.20 (m, 2H), 2.45 (s, 3H), 1.32 (t, J=8.0 Hz, 3H), 1.04 (t, J=8.0 Hz, 3H).

### Step 2

To a solution of N,N-diethyl-2-formyl-5-methyl-benzamide (600 mg, 2.74 mmol, 1 eq) and TMSCF₃ (583.62 mg, 4.10 mmol, 1.5 eq) in THF (5 mL) was added CsF (207.82 mg, 1.37 mmol, 50.44 μL, 0.5 eq) in THF (1 mL) at 0° C. under N₂ atmosphere. The mixture was stirred at 20° C. for 2 hr under N₂ atmosphere. TLC indicated 10% of N,N-diethyl-2-formyl-5-methyl-benzamide was remained, and one major new spot with lower polarity was detected. The residue was diluted with H₂O 5 mL and extracted with EtOAc 30 mL (10 mL*3). The combined organic layers were washed with brine 15 mL (5 mL*3), dried over Na₂SO₄, filtered and concentrated under reduced pressure to give a residue. Compound N,N-diethyl-5-methyl-2-(2,2,2-trifluoro-1-trimethylsilyloxy-ethyl)benzamide (900 mg, crude) was obtained.

### Step 3

To a solution of N,N-diethyl-5-methyl-2-(2,2,2-trifluoro-1-trimethylsilyloxy-ethyl)benzamide (900 mg, 2.49 mmol, 1 eq) in THF (6 mL) was added HCl (2 M, 6.00 mL, 4.82 eq). The mixture was stirred at 20° C. for 0.5 hr under N₂ atmosphere. TLC indicated 10% of N,N-diethyl-5-methyl-2-(2,2,2-trifluoro-1-trimethylsilyloxy-ethyl)benzamide was remained, and two new spots with larger polarity was detected. The residue was diluted with H₂O 10 mL and extracted with DCM 30 mL (10 mL*3). The combined organic layers were washed with brine 15 mL (5 mL*3), dried over Na₂SO₄, filtered and concentrated under reduced pressure to give a residue. The residue was purified by column chromatography (SiO₂, Petroleum ether/Ethyl acetate=20/1 to 2/1). Compound 6-methyl-3-(trifluoromethyl)-3H-isobenzofuran-1-one (200 mg, 925.26 μmol, 37.16% yield) was obtained.

### Step 4

A mixture of 6-methyl-3-(trifluoromethyl)-3H-isobenzofuran-1-one (200 mg, 925.26 μmol, 1 eq), 6-amino-8-methyl-3,4-dihydro-1H-quinolin-2-one (146.74 mg, 832.73 μmol, 0.9 eq) and Al(CH₃)₃ (2 M, 601.42 μL, 1.3 eq) in Tol. (8 mL) was degassed and purged with N₂ for 3 times, and then the mixture was stirred at 100° C. for 2 hr under N₂ atmosphere. LC-MS showed 4.6% of 6-methyl-3-(trifluoromethyl)-3H-isobenzofuran-1-one was remained. Several new peaks were shown on LC-MS and 79.4% of desired compound was detected. The reaction mixture was quenched by poured into 10 mL H₂O and stirred at 0° C. and extracted with EtOAC (10 mL*3). The combined organic layers were washed with brine (10 mL*3), dried over Na₂SO₄, filtered and concentrated under reduced pressure to give a residue. The residue was purified by column chromatography (SiO₂, DCM:MeOH=1:0 to 20/1). Compound 5-methyl-N-(8-methyl-2-oxo-3,4-dihydro-1H-quinolin-6-yl)-2-(2,2,2-trifluoro-1-hydroxy-ethyl)benzamide (320 mg, 815.55 μmol, 88.14% yield) was obtained.

### Step 5

To a solution of 5-methyl-N-(8-methyl-2-oxo-3,4-dihydro-1H-quinolin-6-yl)-2-(2,2,2-trifluoro-1-hydroxy-ethyl)benzamide (320 mg, 815.55 μmol, 1 eq), DMAP (9.96 mg, 81.56 μmol, 0.1 eq) and TEA (247.58 mg, 2.45 mmol, 340.54 μL, 3 eq) in DCM (8 mL) was added 4-methylbenzenesulfonyl chloride (310.97 mg, 1.63 mmol, 2 eq) in DCM (2 mL) at 0° C. The mixture was stirred at 20° C. for 2 hr under N₂ atmosphere. LC-MS showed 4.4% of 5-methyl-N-(8-methyl-2-oxo-3,4-dihydro-1H-quinolin-6-yl)-2-(2,2,2-trifluoro-1-hydroxy-ethyl)benzamide remained. Several new peaks were shown on LC-MS and 84.2% of desired compound was detected. The residue was diluted with H₂O 10 mL and extracted with DCM 45 mL (15 mL*3). The combined organic layers were washed with brine 30 mL (10 mL*3), dried over Na₂SO₄, filtered and concentrated under reduced pressure to give a residue. The residue was purified by column chromatography (SiO₂, Petroleum ether/Ethyl acetate=20/1 to 0/1). Compound [2,2,2-trifluoro-1-[4-methyl-2-[(8-methyl-2-oxo-3,4-dihydro-1H-quinolin-6-yl) carbamoyl]phenyl]ethyl]4-methylbenzenesulfonate (300 mg, 548.89 μmol, 67.30% yield) was obtained. ¹H NMR (400 MHz, CHLOROFORM-d) δ ppm 7.75 (d, J=8.0 Hz, 2H), 7.60 (d, J=8.0 Hz, 1H), 7.20-7.40 (m, 7H), 6.55-6.65 (m, 1H), 3.01 (t, J=8.0 Hz, 2H), 2.60-2.70 (m, 2H), 2.42 (s, 3H), 2.27 (s, 3H), 2.05 (s, 2H).

### Step 6

To a solution of [2,2,2-trifluoro-1-[4-methyl-2-[(8-methyl-2-oxo-3,4-dihydro-1H-quinolin-6-yl) carbamoyl]phenyl]ethyl]4-methylbenzenesulfonate (300 mg, 548.89 μmol, 1 eq) and TEA (166.63 mg, 1.65 mmol, 229.20 μL, 3 eq) in MeOH (10 mL) was added Pd/C (30 mg, 10% purity). The suspension was degassed and purged with H₂ for 3 times. The mixture was stirred under H₂ (45 Psi) at 30° C. for 2 hr. LC-MS showed 34.1% of [2,2,2-trifluoro-1-[4-methyl-2-[(8-methyl-2-oxo-3,4-dihydro-1H-quinolin-6-yl) carbamoyl]phenyl]ethyl]4-methylbenzenesulfonate remained. Several new peaks were shown on LC-MS and 36.2% of desired compound was detected. The reaction mixture was filtered and filter liquor was concentrated under reduced pressure to give a residue. The residue was purified by prep-TLC (SiO₂, DCM:MeOH=20:1). Compound 5-methyl-N-(8-methyl-2-oxo-3,4-dihydro-1H-quinolin-6-yl)-2-(2,2,2-trifluoroethyl)benzamide (44 mg, 110.48 μmol, 20.13% yield) was obtained. (M+H)⁺: 377.2. ¹H NMR (400 MHz, METHANOL-d4) δ ppm 7.43 (s, 1H), 7.39 (s, 1H), 7.34 (t, J=12.0 Hz, 3H), 3.75-7.85 (m, 2H), 2.96 (t, J=4.0 Hz, 2H), 2.50-2.60 (m, 2H), 2.42 (s, 3H), 2.27 (s, 3H).

### Example 6: Synthesis of 3-ethyl-5-fluoro-N-(8-methyl-2-oxo-1,2,3,4-tetrahydroquinolin-6-yl)isonicotinamide (Compound 6)

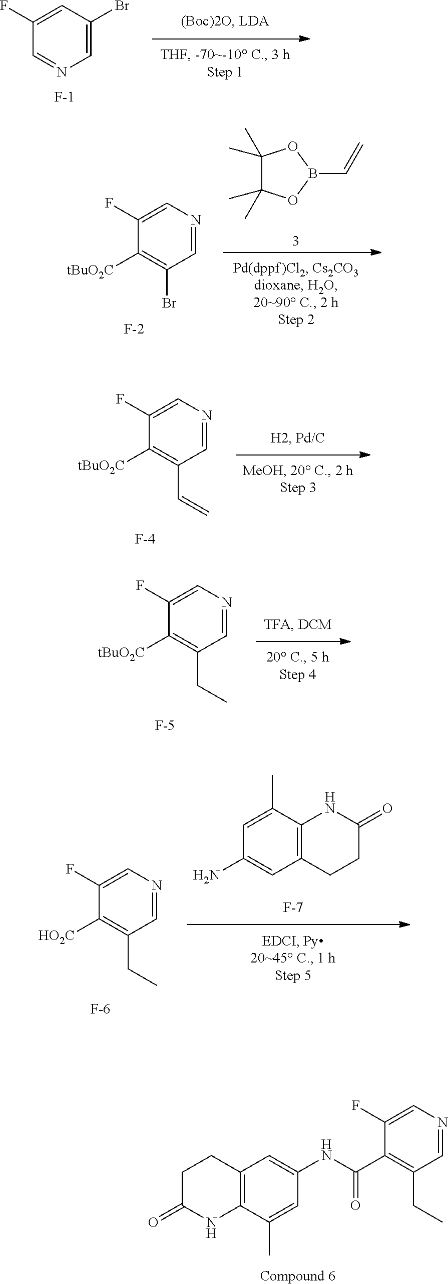

### Step 1

To a solution LDA (2 M, 17.05 mL, 1.2 eq) in THF (100 mL) was added 3-bromo-5-fluoro-pyridine (5 g, 28.41 mmol, 1 eq) in THF (30 mL) dropwise at −70° C. Then the reaction mixture was stirred at −70° C. for 1 hr. Then to the mixture was added (Boc)₂O (12.40 g, 56.82 mmol, 13.05 mL, 2 eq) in THF (30 mL) dropwise at −10° C. The final mixture was stirred at −10° C. for 2 hr. LC-MS showed 3-bromo-5-fluoro-pyridine was consumed completely. TLC indicated 3-bromo-5-fluoro-pyridine was consumed completely and one new spot formed. Combined with another batch (5 g scale). The reaction mixture was quenched by addition H₂O 200 mL at 0° C., and then diluted with H₂O 50 mL and extracted with EtOAc (200 mL*2). The combined organic layers were washed with brine (100 mL*3), dried over Na₂SO₄, filtered and concentrated under reduced pressure to give a residue. The residue was purified by column chromatography (SiO₂, Petroleum ether/Ethyl acetate=100/1 to 10/1). Compound tert-butyl 3-bromo-5-fluoro-pyridine-4-carboxylate (3.6 g, 13.04 mmol) was obtained.

### Step 2

To the mixture of tert-butyl 3-bromo-5-fluoro-pyridine-4-carboxylate (500 mg, 1.81 mmol, 1 eq) and 4,4,5,5-tetramethyl-2-vinyl-1,3,2-dioxaborolane (334.69 mg, 2.17 mmol, 368.60 μL, 1.2 eq) in H₂O (0.5 mL) and dioxane (5 mL) was added Cs₂CO₃ (1.77 g, 5.43 mmol, 3 eq) and Pd(dppf)Cl₂ (132.51 mg, 181.09 μmol, 0.1 eq) at 20° C. Then the reaction mixture was stirred at 90° C. for 2 hr. TLC indicated tert-butyl 3-bromo-5-fluoro-pyridine-4-carboxylate was consumed completely and one new spot formed. The reaction mixture was diluted with H₂O 50 mL and extracted with ethyl acetate (10 mL*3). The combined organic layers were washed with brine 15 mL (5 mL*3), dried over Na₂SO₄, filtered and concentrated under reduced pressure to give a residue. The residue was purified by column chromatography (SiO₂, Petroleum ether/Ethyl acetate=100/1 to 9/1). Compound tert-butyl 3-fluoro-5-vinyl-pyridine-4-carboxylate (230 mg, 1.03 mmol, 56.89% yield) was obtained. ¹H NMR (400 MHz, CHLOROFORM-d) δ ppm 8.62 (s, 1H), 8.42 (s, 1H), 6.75-6.85 (m, 1H), 5.87 (d, J=16.0 Hz, 1H), 5.54 (d, J=12.0 Hz, 1H), 1.62 (s, 9H).

### Step 3

To a solution of tert-butyl 3-fluoro-5-vinyl-pyridine-4-carboxylate (200 mg, 895.89 μmol, 1 eq) in MeOH (10 mL) was added Pd/C (20 mg, 10% purity) under Ar atmosphere. The suspension was degassed and purged with H₂ for 3 times. The mixture was stirred under H₂ (15 Psi) at 20° C. for 2 hr. LC-MS showed tert-butyl 3-fluoro-5-vinyl-pyridine-4-carboxylate was consumed completely and one main peak with desired m/z was detected. The reaction mixture was filtered and the filter liquor was concentrated under reduced pressure to give a residue. Compound tert-butyl 3-ethyl-5-fluoro-pyridine-4-carboxylate (200 mg, 887.87 μmol, 99.11% yield) was obtained.

### Step 4

A mixture of tert-butyl 3-ethyl-5-fluoro-pyridine-4-carboxylate (100 mg, 443.93 μmol, 1 eq) and TFA (50.62 mg, 443.93 μmol, 32.87 μL, 1 eq) in DCM (2 mL) was degassed and purged with N₂ for 3 times. Then the reaction mixture was stirred at 20° C. for 5 hr. LC-MS showed tert-butyl 3-ethyl-5-fluoro-pyridine-4-carboxylate was consumed completely and one main peak with desired m/z was detected. The reaction mixture was concentrated under reduced pressure to give a residue. Compound 3-ethyl-5-fluoro-pyridine-4-carboxylic acid (80 mg, crude, TFA) was obtained.

### Step 5

To the mixture of 3-ethyl-5-fluoro-pyridine-4-carboxylic acid (70 mg, 247.20 μmol, 1.05 eq, TFA) and 6-amino-8-methyl-3,4-dihydro-1H-quinolin-2-one (41.49 mg, 235.43 μmol, 1 eq) in Py (3 mL) was added EDCI (54.16 mg, 282.51 μmol, 1.2 eq) at 20° C. Then the reaction mixture was stirred at 45° C. for 1 hr. LC-MS showed 3-ethyl-5-fluoro-pyridine-4-carboxylic acid was consumed completely. Several new peaks were shown on LC-MS and 8% of desired compound was detected. The reaction mixture was filtered and filter liquor was concentrated under reduced pressure to give a residue. The residue was purified by prep-TLC (SiO₂, petroleum ether:ethyl acetate=0:1). Compound 3-ethyl-5-fluoro-N-(8-methyl-2-oxo-3,4-dihydro-1H-quinolin-6-yl)pyridine-4-carboxamide (15 mg, 45.36 μmol, 19.27% yield) was obtained. (M+H)⁺: 328.2. ¹H NMR (400 MHz, METHANOL-d4) δ ppm 8.43 (d, J=8.0 Hz, 2H), 7.41 (s, 1H), 7.33 (t, J=8.0 Hz, 1H), 2.96 (t, J=8.0 Hz, 2H), 2.75-2.85 (m, 2H), 2.58 (t, J=8.0 Hz, 2H), 2.28 (s, 3H), 1.29 (t, J=8.0 Hz, 3H).

### Example 7: Synthesis of 3-amino-5-ethyl-N-(8-methyl-2-oxo-1,2,3,4-tetrahydroquinolin-6-yl)isonicotinamide (Compound 8)

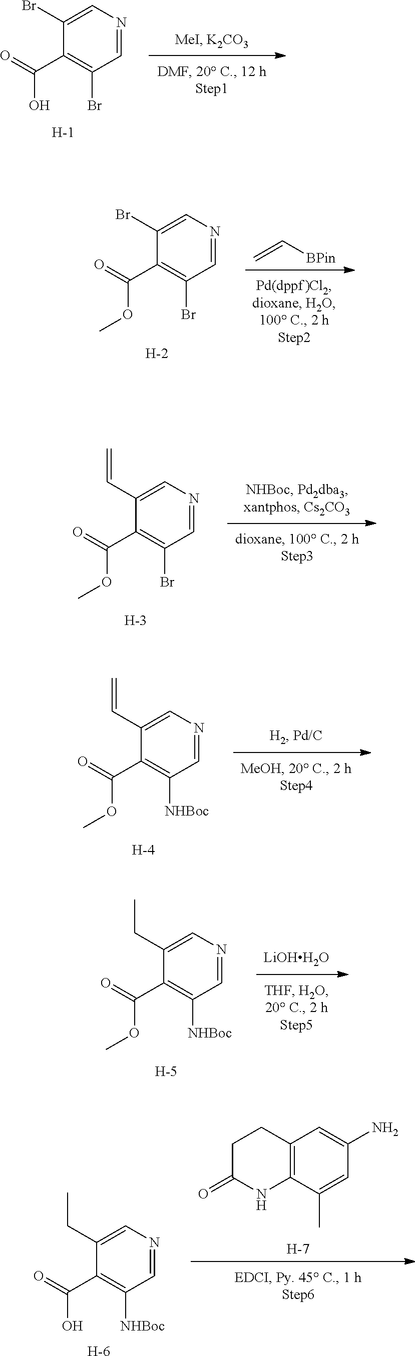

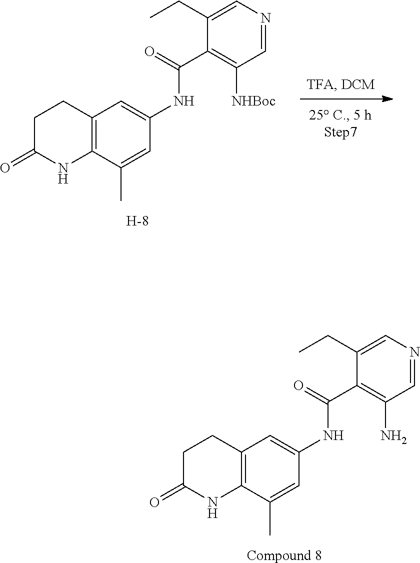

### Step 1

To the mixture of 3,5-dibromopyridine-4-carboxylic acid (14 g, 49.84 mmol, 1 eq) and K₂CO₃ (13.78 g, 99.68 mmol, 2 eq) in DMF (140 mL) was added MeI (8.49 g, 59.81 mmol, 3.72 mL, 1.2 eq). The mixture was stirred at 20° C. for 12 hr. LC-MS showed the starting material was consumed completely. The reaction mixture was diluted with H₂O 700 mL and extracted with EtOAc (300 mL*4). The combined organic layers were washed with brine (150 mL*3), dried over anhydrous Na₂SO₄, filtered and concentrated under reduced pressure to give a residue. The residue was purified by column chromatography (SiO₂, Petroleum ether/Ethyl acetate=20/1 to 0/1). The product of methyl 3,5-dibromopyridine-4-carboxylate (12 g, 40.69 mmol, 81.64% yield) was obtained. (M+H): 295.9. ¹H NMR (400 MHz, DMSO-d6) δ ppm 8.88 (s, 2H) 3.97 (s, 3H)

### Step 2

To the solution of methyl 3,5-dibromopyridine-4-carboxylate (5 g, 16.95 mmol, 1 eq) and 4,4,5,5-tetramethyl-2-vinyl-1,3,2-dioxaborolane (2.61 g, 16.95 mmol, 2.88 mL, 1 eq), Cs₂CO₃ (11.05 g, 33.91 mmol, 2 eq) in dioxane (80 mL) and H₂O (8 mL) was added Pd(dppf)Cl₂ (1.24 g, 1.70 mmol, 0.1 eq). The mixture was stirred at 100° C. for 2 hr. LC-MS showed methyl 3,5-dibromopyridine-4-carboxylate was consumed completely. The reaction mixture was diluted with H₂O 80 mL and extracted with EtOAc (50 mL*4). The combined organic layers were washed with brine (30 mL*3), dried over anhydrous Na₂SO₄, filtered and concentrated under reduced pressure to give a residue. The residue was purified by column chromatography (SiO₂, Petroleum ether/Ethyl acetate=30/1 to 0/1). Compound methyl 3-bromo-5-vinyl-pyridine-4-carboxylate (1.5 g, 5.70 mmol, 33.63% yield) was obtained. (M+H): 242.0. ¹H NMR (400 MHz, DMSO-d6) δ ppm 8.94 (s, 1H), 8.77 (s, 1H), 6.60 (m, 1H), 6.06 (d, J=17.6 Hz, 1H), 5.57 (d, J=11.2 Hz, 1H), 3.94 (s, 3H).

### Step 3

A mixture of methyl 3-bromo-5-vinyl-pyridine-4-carboxylate (1 g, 4.13 mmol, 1 eq), tert-butyl carbamate (967.87 mg, 8.26 mmol, 2 eq), Cs₂CO₃ (2.69 g, 8.26 mmol, 2 eq), Pd₂(dba)₃ (378.29 mg, 413.11 μmol, 0.1 eq) and Xantphos (239.03 mg, 413.11 μmol, 0.1 eq) in dioxane (20 mL) was degassed and purged with N₂ for 3 times, and then the mixture was stirred at 100° C. for 2 hr under N₂ atmosphere. LC-MS showed methyl 3-bromo-5-vinyl-pyridine-4-carboxylate was consumed completely and one main peak with desired mass was detected. The reaction mixture was concentrated under reduced pressure to give a residue. The residue was purified by column chromatography (SiO₂, Petroleum ether/Ethyl acetate=10/1 to 0/1). Compound methyl 3-(tert-butoxycarbonylamino)-5-vinyl-pyridine-4-carboxylate (1.5 g, 4.96 mmol, 55.20% yield) was obtained. (M+H): 279.2. ¹H NMR (400 MHz, DMSO-d6) δ ppm 9.40 (s, 1H), 8.62 (s, 1H), 8.45 (s, 1H), 6.79-6.87 (m, 1H), 5.89 (d, J=17.6 Hz, 1H), 5.45 (d, J=11.2 Hz, 1H), 3.79 (s, 3H), 1.44 (s, 9H).

### Step 4

To the solution of Pd/C (0.2 g, 10% purity) in MeOH (20 mL) was added methyl 3-(tert-butoxycarbonylamino)-5-vinyl-pyridine-4-carboxylate (1.5 g, 5.39 mmol, 1 eq) under N₂. The suspension was degassed under vacuum and purged with H₂ several times. The mixture was stirred under H₂ (15 psi) at 20° C. for 2 hours. LC-MS showed the starting material was consumed completely and one main peak with desired mass was detected. The reaction mixture was filtered and concentrated under reduced pressure to give a residue. Compound methyl 3-(tert-butoxycarbonylamino)-5-ethyl-pyridine-4-carboxylate (1.5 g, 4.71 mmol, 87.37% yield) was obtained. (M+H): 281.2. ¹H NMR (400 MHz, DMSO-d6) δ ppm 9.36 (br s, 1H), 8.36 (s, 1H), 8.30 (s, 1H), 3.79 (s, 3H), 2.62-2.69 (m, 2H), 1.44 (s, 9H), 1.13 (t, J=7.6 Hz, 3H).

### Step 5

To the mixture of methyl 3-(tert-butoxycarbonylamino)-5-ethyl-pyridine-4-carboxylate (1.5 g, 5.35 mmol, 1 eq) in THF (10 mL) and H₂O (10 mL) was added LiOH·H₂O (449.10 mg, 10.70 mmol, 2 eq). The mixture was stirred at 20° C. for 2 hr. LC-MS showed the starting material was consumed completely and one main peak with desired mass was detected. The reaction mixture was concentrated under reduced pressure to remove THF. The reaction mixture was adjusted to pH=7 by 1N HCl solution. The reaction mixture was filtered and filter cake concentrated under reduced pressure to give a residue. Compound 3-(tert-butoxycarbonylamino)-5-ethyl-pyridine-4-carboxylic acid (700 mg, 2.52 mmol, 47.16% yield) was obtained. (M+H): 267.2. ¹H NMR (400 MHz, METHANOL-d4) δ ppm 9.10 (s, 1H), 8.30 (s, 1H), 2.88-2.96 (m, 2H), 1.53 (s, 9H), 1.27 (t, J=7.6 Hz, 3H).

### Step 6

A mixture of 3-(tert-butoxycarbonylamino)-5-ethyl-pyridine-4-carboxylic acid (300 mg, 990.91 μmol, 1 eq, HCl), 6-amino-8-methyl-3,4-dihydro-1H-quinolin-2-one (174.61 mg, 990.91 μmol, 1 eq), EDCI (227.95 mg, 1.19 mmol, 1.2 eq) in Py (2 mL) was degassed and purged with N₂ for 3 times, and then the mixture was stirred at 45° C. for 1 hr under N₂ atmosphere. The reaction mixture was concentrated under reduced pressure to give a residue. The residue was purified by column chromatography (SiO₂, Petroleum ether/Ethyl acetate=30/1 to 0/1). Compound tert-butyl N-[5-ethyl-4-[(8-methyl-2-oxo-3,4-dihydro-1H-quinolin-6-yl) carbamoyl]-3-pyridyl]carbamate (120 mg, 282.69 μmol, 28.53% yield) was obtained. (M+H): 425.3. ¹H NMR (400 MHz, METHANOL-d4) δ ppm 8.69 (s, 1H), 8.31 (s, 1H), 7.42 (s, 1H), 6.46 (s, 1H), 2.92-2.98 (m, 2H), 2.74-2.80 (m, 2H), 2.57 (m, 2H), 2.27 (s, 3H), 1.46 (s, 9H), 1.27 (t, J=7.6 Hz, 3H).

### Step 7

A mixture of tert-butyl N-[5-ethyl-4-[(8-methyl-2-oxo-3,4-dihydro-1H-quinolin-6-yl) carbamoyl]-3-pyridyl]carbamate (120 mg, 282.69 μmol, 1 eq) in TFA (5 mL) and DCM (5 mL) was degassed and purged with N₂ for 3 times, and then the mixture was stirred at 25° C. for 5 hr under N₂ atmosphere. LC-MS showed the starting material was consumed completely and one main peak with desired mass was detected. The reaction mixture was concentrated under reduced pressure to give a residue. The residue was adjusted to pH=7 by saturated NaHCO₃ solution. The solution was extracted with EtOAc (2 mL*3). The combined organic layers were washed with brine (1 mL*2), dried over anhydrous Na₂SO₄, filtered and concentrated under reduced pressure to give a residue. Compound 3-amino-5-ethyl-N-(8-methyl-2-oxo-3,4-dihydro-1H-quinolin-6-yl)pyridine-4-carboxamide (100 mg, crude) was obtained. (M+H): 325.2. ¹H NMR (400 MHz, METHANOL-d4) δ ppm 7.96 (s, 1H), 7.78 (s, 1H), 7.43 (s, 1H), 7.36 (s, 1H), 2.96 (t, J=7.6 Hz, 2H), 2.65-2.70 (m, 2H), 2.57 (t, J=7.6 Hz, 2H), 2.27 (s, 3H), 1.22-1.29 (m, 3H).

### Example 8: Synthesis of N-(8-(1-methyl-1H-imidazol-4-yl)-2-oxo-1,2,3,4-tetrahydroquinolin-6-yl)-3-(2,2,2-trifluoroethyl)isonicotinamide (Compound 11)

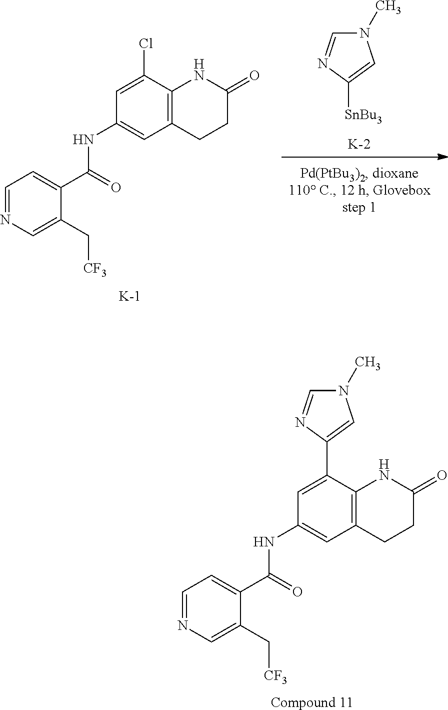

### Step 1

To a solution of N-(8-chloro-2-oxo-3,4-dihydro-1H-quinolin-6-yl)-3-(2,2,2-trifluoroethyl)pyridine-4-carboxamide (100 mg, 260.59 μmol, 1 eq), tributyl-(1-methylimidazol-4-yl) stannane (145.07 mg, 390.88 μmol, 1.5 eq) in dioxane (3 mL) was added palladium;tritert-butylphosphane (13.32 mg, 26.06 μmol, 0.1 eq) under glovebox. The mixture was stirred at 110° C. for 12 hr. LC-MS showed starting reactant remained and desired compound was detected. The mixture was diluted with H₂O 10 mL and extracted with EtOAc 20 mL (10 mL*2). The combined organic layers were dried over Na₂SO₄, filtered and concentrated under reduced pressure to give a residue. The residue was purified by prep-TLC (SiO₂, EtOAc:MeOH=5:1). Compound N-[8-(1-methylimidazol-4-yl)-2-oxo-3,4-dihydro-1H-quinolin-6-yl]-3-(2,2,2-trifluoroethyl)pyridine-4-carboxamide (60 mg, 138.75 μmol, 53% yield) was obtained. ¹H NMR (400 MHz, DMSO-d₆) δ ppm 11.75 (s, 1H), 10.60 (s, 1H), 8.68-8.80 (m, 2H), 7.88 (s, 1H), 7.82-7.86 (m, 1H), 7.63 (d, J=4.8 Hz, 1H), 7.58 (s, 1H), 7.36-7.42 (m, 1H), 4.01 (q, J=11.2 Hz, 2H), 3.76 (s, 3H), 2.93 (t, J=7.2 Hz, 2H), 2.43-2.50 (m, 2H). (M+H)⁺: 430.2.

### Example 9: Synthesis of methyl 2-oxo-6-(3-(2,2,2-trifluoroethyl) isonicotinamido)-1,2,3,4-tetrahydroquinoline-8-carboxylate (Compound 12) and 2-oxo-6-(3-(2,2,2-trifluoroethyl)isonicotinamido)-1,2,3,4-tetrahydroquinoline-8-carboxamide (Compound 13)

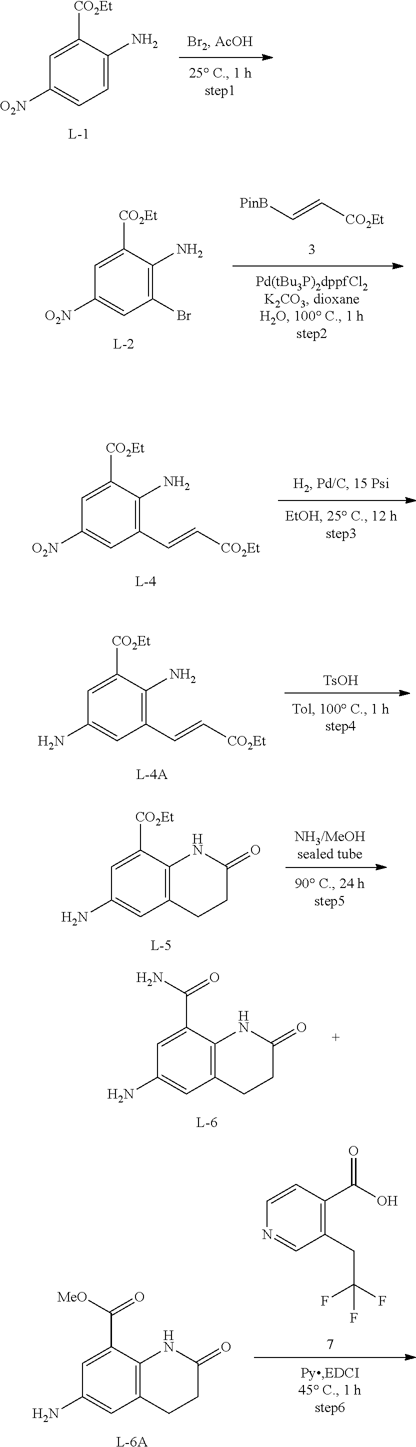

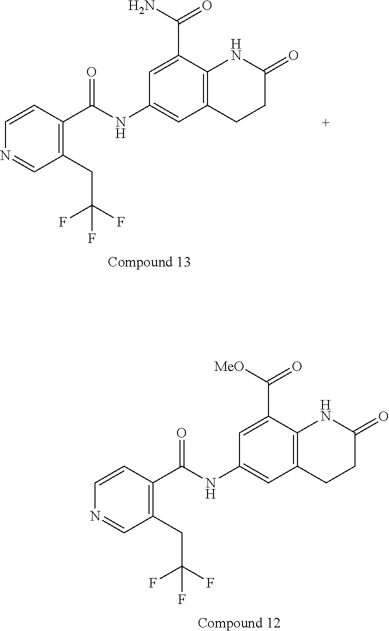 [SMILES: NC(=O)c1cc(NC(=O)c2ccncc2CC(F)(F)F)cc2c1NC(=O)CC2 ; "Compound 13" ; COC(=O)c1cc(NC(=O)c2ccncc2CC(F)(F)F)cc2c1NC(=O)CC2 ; "Compound 12"]

### Step 1

Two batches in parallel. To a solution of ethyl 2-amino-5-nitro-benzoate (5 g, 23.79 mmol, 1 eq) in AcOH (50 mL) was added Br₂ (4.94 g, 30.92 mmol, 1.59 mL, 1.3 eq). The mixture was stirred at 25° C. for 1 hr. LC-MS showed the starting material was consumed completely and one main peak with desired mass was detected. The reaction mixture was poured into ice-water (100 mL) slowly, filtered and the filter cake was concentrated under reduced pressure to give a residue. Compound ethyl 2-amino-3-bromo-5-nitro-benzoate (13 g, 44.97 mmol, 94.52% yield) was obtained. ¹H NMR (400 MHz, METHANOL-d₄) δ ppm 8.76 (d, J=2.8 Hz, 1H), 8.49 (d, J=2.8 Hz, 1H), 4.38-4.45 (m, 2H), 1.42 (t, J=7.2 Hz, 3H).

### Step 2

To a solution of ethyl 2-amino-3-bromo-5-nitro-benzoate (3 g, 10.38 mmol, 1 eq) and ethyl (E)-3-(4,4,5,5-tetramethyl-1,3,2-dioxaborolan-2-yl) prop-2-enoate (3.52 g, 15.57 mmol, 1.5 eq) in dioxane (30 mL) and H₂O (3 mL) was added ditert-butyl(cyclopenta-1,4-dien-1-yl)phosphane; dichloropalladium;iron (676.36 mg, 1.04 mmol, 0.1 eq) and K₂CO₃ (2.87 g, 20.76 mmol, 2 eq). The mixture was stirred at 100° C. for 1 hr. LC-MS showed ethyl 2-amino-3-bromo-5-nitro-benzoate was consumed completely and one main peak with desired mass was detected. The reaction mixture was diluted with H₂O 30 mL and extracted with EtOAc 150 mL (50 mL*3). The combined organic layers were washed with brine 80 mL (40 mL*2), dried over Na₂SO₄, filtered and concentrated under reduced pressure to give a residue. The residue was purified by column chromatography (SiO₂, Petroleum ether/Ethyl acetate=1/0 to 1/1). Compound ethyl 2-amino-3-[(E)-3-ethoxy-3-oxo-prop-1-enyl]-5-nitro-benzoate (2.6 g, 8.35 mmol, 80.46% yield) was obtained.

### Step 3

To the solution of Pd/C (200 mg, 10% purity) in EtOH (30 mL) was added ethyl 2-amino-3-[(E)-3-ethoxy-3-oxo-prop-1-enyl]-5-nitro-benzoate (2 g, 6.49 mmol, 1 eq) under N₂. The suspension was degassed under vacuum and purged with H₂ 3 times. The mixture was stirred under H₂ (15 Psi) at 25° C. for 12 hr. TLC indicated the starting material was consumed completely and one new spot formed. The reaction mixture was filtered and concentrated under reduced pressure to give a residue. Compound ethyl 2, 5-diamino-3-(3-ethoxy-3-oxo-propyl)benzoate (1.6 g, 5.71 mmol, 87.98% yield) was obtained.

### Step 4

To a solution of ethyl 2, 5-diamino-3-(3-ethoxy-3-oxo-propyl)benzoate (1.6 g, 5.71 mmol, 1 eq) in Tol. (16 mL) was added TsOH (98.29 mg, 570.78 μmol, 0.1 eq). The mixture was stirred at 100° C. for 1 hr. The reaction mixture was diluted with H₂O 20 mL and extracted with EtOAc 120 mL (40 mL*3). The combined organic layers were washed with brine 80 mL (40 mL*2), dried over Na₂SO₄, filtered and concentrated under reduced pressure to give a residue. Compound ethyl 6-amino-2-oxo-3,4-dihydro-1H-quinoline-8-carboxylate (total 1.5 g, 91% yield) was obtained. 1H NMR (400 MHz, DMSO-d₆) δ ppm 9.82 (s, 1H), 7.04 (d, J=2.4 Hz, 1H), 6.70-6.77 (m, 1H), 5.09 (s, 2H), 4.25-4.35 (m, 2H), 2.82 (t, J=7.2 Hz, 2H), 2.42-2.47 (m, 2H), 1.29-1.37 (m, 3H).

### Step 5

A solution of ethyl 6-amino-2-oxo-3,4-dihydro-1H-quinoline-8-carboxylate (200 mg, 853.79 μmol, 1 eq) in NH₃/MeOH (7 M, 10.00 mL, 81.99 eq) was stirred at 90° C. for 24 hr. LC-MS showed the starting material was consumed completely and 6-amino-2-oxo-3,4-dihydro-1H-quinoline-8-carboxamide as main peak with desired mass was detected. The mixture was cooled 25° C. and concentrated under reduced pressure to give a residue. Compound 6-amino-2-oxo-3,4-dihydro-1H-quinoline-8-carboxamide (140 mg, 682.22 μmol, 79.91% yield) and methyl 6-amino-2-oxo-3,4-dihydro-1H-quinoline-8-carboxylate (80 mg, 363.27 μmol, 42.55% yield) were obtained.

### Step 6

To a solution of methyl 6-amino-2-oxo-3,4-dihydro-1H-quinoline-8-carboxylate and 6-amino-2-oxo-3,4-dihydro-1H-quinoline-8-carboxamide (200 mg) and 3-(2,2,2-trifluoroethyl)pyridine-4-carboxylic acid (224.80 mg, 930.50 μmol, 1.5 eq, HCl) in Py (2 mL) was added EDCI (178.38 mg, 930.50 μmol, 1.5 eq). The mixture was stirred at 45° C. for 1 hr. LC-MS showed 6-amino-2-oxo-3,4-dihydro-1H-quinoline-8-carboxamide was consumed completely and 2-oxo-6-[[3-(2,2,2-trifluoroethyl)pyridine-4-carbonyl]amino]-3,4-dihydro-1H-quinoline-8-carboxamide as main peak with desired mass was detected. The reaction mixture was filtered and concentrated under reduced pressure to give a residue. The residue was purified by prep-HPLC (column: WePure Biotech XPt C_{18150*40*7} μm; mobile phase: [H₂O (10 mM NH₄HCO₃)-ACN]; gradient: 20%-60% B over 8.0 min).

Compound 13 2-oxo-6-[[3-(2,2,2-trifluoroethyl)pyridine-4-carbonyl]amino]-3,4-dihydro-1H-quinoline-8-carboxamide (84 mg, 205.75 μmol, 33.17% yield) was obtained. ¹H NMR (400 MHz, METHANOL-d₄) δ ppm 8.66-8.75 (m, 2H), 7.96 (d, J=2.0 Hz, 1H), 7.66 (d, J=4.8 Hz, 1H), 7.58-7.63 (m, 1H), 3.90-4.03 (m, 2H), 3.05 (t, J=7.2 Hz, 2H), 2.56-2.69 (m, 2H). (M+H)⁺: 393.1.

Compound 12 methyl 2-oxo-6-[[3-(2,2,2-trifluoroethyl)pyridine-4-carbonyl]amino]-3,4-dihydro-1H-quinoline-8-carboxylate (45 mg, 104.29 μmol, 16.81% yield) was obtained. ¹H NMR (400 MHz, METHANOL-d₄) δ ppm 8.65-8.74 (m, 2H), 8.25 (d, J=2.4 Hz, 1H), 7.76-7.83 (m, 1H), 7.65 (d, J=4.8 Hz, 1H), 3.88-4.03 (m, 5H), 3.07 (t, J=7.2 Hz, 2H), 2.63-2.69 (m, 2H). (M+H)⁺: 408.1.

### Example 10: Synthesis of N-(8-(4-methyl-1H-pyrazol-1-yl)-2-oxo-1,2,3,4-tetrahydroquinolin-6-yl)-3-(2,2,2-trifluoroethyl)isonicotinamide (Compound 14)

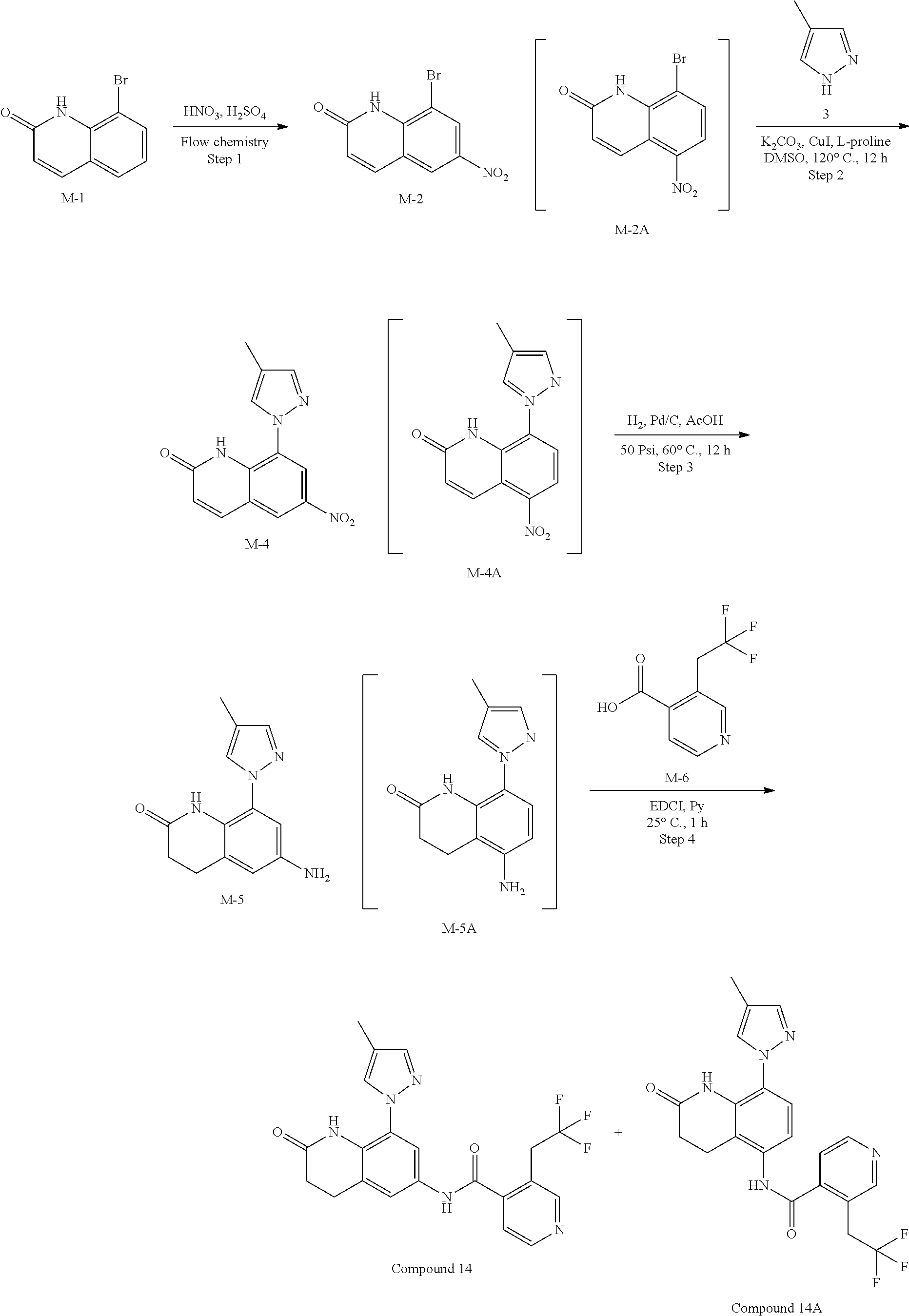

Solution 1: {8-bromo-1H-quinolin-2-one, 1 eq,3 g} in {H₂SO₄, 15 mL}

Solution 2: {Fuming Nitric Acid, 1.2 eq, 1.012 g} in {H₂SO₄, 15 mL}.

The solution 1 was pumped by Pump 1 {S1, P1, 2.969 mL/min} to flow reactor 1 {FLR1, PFA, Coils reactor, 3.175 (⅛″) mm, 30 mL, 0° C.}.

The solution 2 was pumped by Pump 2 {S2, P2, 3.031 mL/min} to flow reactor 1 {FLR1, PFA, Coils reactor, 3.175 (⅛″) mm, 30 mL, 0° C.}.

The residence time of flow reactor 1 was {FLR1, 5 min}.

The mixture was collected with a bottle.

The Pump 1 and Pump 2 was started at the same time. The reaction mixture was collected after running 1 min. Stop collecting the reaction mixture after 10 mins. The mixture was quenched by ice water (100 mL) and filtered. The filter cake was collected. The mixture of 8-bromo-6-nitro-1H-quinolin-2-one and 8-bromo-5-nitroquinolin-2 (1H)-one (3 g, 11.15 mmol) was obtained.

### Step 2

A mixture of 8-bromo-6-nitro-1H-quinolin-2-one and 8-bromo-5-nitroquinolin-2(1H)-one (2 g, 7.43 mmol, 1 eq) was added to the solution of 4-methyl-1H-pyrazole (4.88 g, 59.47 mmol, 4.78 mL, 8 eq), K₂CO₃ (2.05 g, 14.87 mmol, 2 eq), CuI (1.42 g, 7.43 mmol, 1 eq) and L-proline (427.91 mg, 3.72 mmol, 0.5 eq) in DMSO (30 mL). The mixture was stirred at 120° C. for 12 hrs under N₂ atmosphere. The reaction mixture was filtered and extracted with Ethyl acetate (50 mL*3). The combined organic layers were washed with Saturated NaCl solution (20 mL*2), dried over Na₂SO₄, filtered and concentrated under reduced pressure to give a residue. The residue was purified by column chromatography (SiO₂, Petroleum ether/Ethyl acetate=1/1 to 1/1). A mixture of 8-(4-methylpyrazol-1-yl)-6-nitro-1H-quinolin-2-one and 8-(4-methyl-1H-pyrazol-1-yl)-5-nitroquinolin-2 (1H)-one (0.5 g, 1.85 mmol, 24.89% yield) was obtained.

### Step 3

To a solution of the mixture 8-(4-methylpyrazol-1-yl)-6-nitro-1H-quinolin-2-one (0.25 g, 925.09 μmol, 1 eq) in MeOH (50 mL) was added Pd/C (492.24 mg, 462.55 μmol, 10% purity, 0.5 eq) under N₂ atmosphere. The suspension was degassed and purged with H₂ for 3 times. The mixture was stirred under H₂ (50 Psi.) at 60° C. for 12 hrs. LC-MS showed the starting material was consumed completely and two desired mass was detected. The reaction mixture was filtered and concentrated under reduced pressure to give a residue. The mixture of 6-amino-8-(4-methylpyrazol-1-yl)-3,4-dihydro-1H-quinolin-2-one and 5-amino-8-(4-methyl-1H-pyrazol-1-yl)-3,4-dihydroquinolin-2 (1H)-one (400 mg, crude) was obtained.

### Step 4

A mixture of 6-amino-8-(4-methylpyrazol-1-yl)-3,4-dihydro-1H-quinolin-2-one and 5-amino-8-(4-methyl-1H-pyrazol-1-yl)-3,4-dihydroquinolin-2 (1H)-one (80 mg, 330.20 μmol, 1 eq), 3-(2,2,2-trifluoroethyl) isonicotinic acid (67.74 mg, 330.20 μmol, 1 eq), EDCI (75.96 mg, 396.24 μmol, 1.2 eq), 3-(2,2,2-trifluoroethyl) isonicotinic acid (67.74 mg, 330.20 μmol, 1 eq) and Py (5 mL) was degassed and purged with N₂ for 3 times, and then the mixture was stirred at 25° C. for 1 hr under N₂ atmosphere. LC-MS showed the starting material was consumed completely and two desired mass was detected. Concentrated under reduced pressure to give a residue. The residue was purified by prep-HPLC (column: CD04-Welch Utimate C18 150*25*7 μm; mobile phase: [water (FA)-ACN]; gradient: 14%-44% B over 15 min). Two targets were obtained.

Compound 14A: (0.02 g, 44.82 μmol, 6.79% yield) was obtained. (M+H)⁺: 430.0. ¹H NMR (400 MHz, DMSO-d6) δ ppm 10.50 (s, 1H), 10.34 (s, 1H), 8.67-8.83 (m, 2H), 8.16 (s, 1H), 7.74 (br s, 2H), 7.49 (br d, J=8.4 Hz, 1H), 7.15 (d, J=8.8 Hz, 1H), 4.02 (q, J=11.2 Hz, 2H), 2.91-2.99 (m, 2H), 2.27-2.46 (m, 2H), 2.13 (s, 3H).

Compound 14: (0.02 g, 45.77 μmol, 6.93% yield) was obtained. (M+H)⁺: 430.0. ¹H NMR (400 MHz, DMSO-d6) δ ppm 10.77 (s, 1H), 10.03 (s, 1H), 8.70-8.80 (m, 2H), 8.01 (s, 1H), 7.71-7.83 (m, 2H), 7.63 (d, J=4.8 Hz, 1H), 7.49 (s, 1H), 4.00 (q, J=11.2 Hz, 2H), 3.00 (t, J=7.2 Hz, 2H), 2.50-2.54 (m, 2H), 2.13 (s, 3H)

### Example 11: Synthesis of N-(8-fluoro-2-oxo-1,2,3,4-tetrahydroquinolin-6-yl)-3-(2,2,2-trifluoroethyl)isonicotinamide (Compound 15)

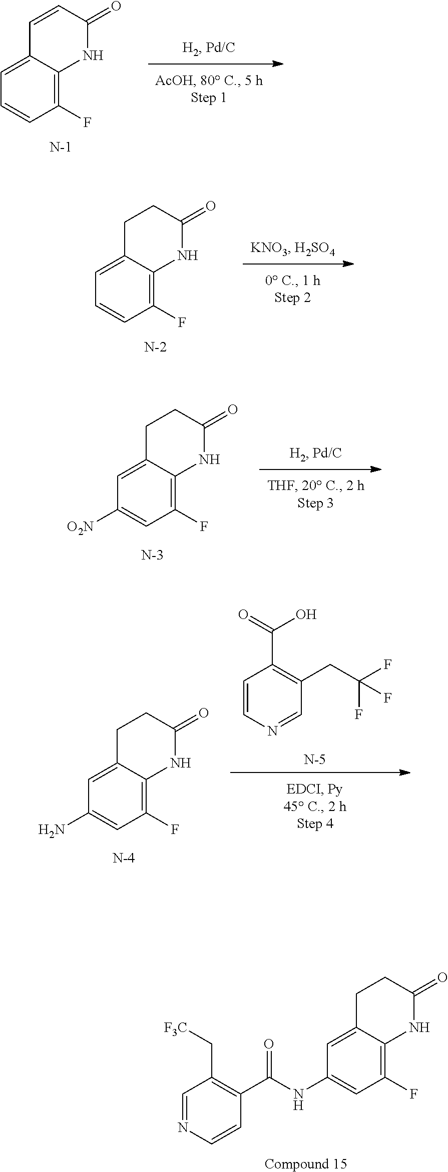

### Step 1

To a solution of Pd/C (100 mg, 6.13 mmol, 10% purity) in AcOH (10 mL) was added 8-fluoro-1H-quinolin-2-one (1 g, 6.13 mmol, 1 eq) under Ar atmosphere. The suspension was degassed and purged with H₂ for 2 times. The mixture was stirred under H₂ (50 Psi.) at 80° C. for 5 hr. LC-MS showed 8-fluoro-1H-quinolin-2-one was consumed completely and one main peak with desired m/z was detected. The reaction mixture was filtered and concentrated under reduced pressure to give a residue. Compound 8-fluoro-3,4-dihydro-1H-quinolin-2-one (800 mg, crude) was obtained.

### Step 2

To a 100 mL three neck bottle was added the solution of 8-fluoro-3,4-dihydro-1H-quinolin-2-one (400 mg, 2.42 mmol, 1 eq) in H₂SO₄ (5 mL). Then to the mixture was added KNO₃ (340 mg, 3.36 mmol, 1.39 eq) in portions at 0° C. The mixture was stirred at 0° C. for 1 hr. LC-MS showed 8-fluoro-3,4-dihydro-1H-quinolin-2-one was consumed completely and one main peak with desired m/z was detected. The mixture was poured into ice (15 mL) slowly. The reaction mixture was diluted with H₂O (10 mL) and extracted with ethyl acetate (5 mL*3). The combined organic layers were washed with brine (5 mL*3), dried over Na₂SO₄, filtered and concentrated under reduced pressure to give a residue. Compound 8-fluoro-6-nitro-3,4-dihydro-1H-quinolin-2-one (300 mg, crude) was obtained. ¹H NMR (400 MHz, DMSO-d₆) δ ppm 10.74 (s, 1H), 8.03 (t, J=8.0 Hz, 2H), 3.09 (t, J=8.0 Hz, 2H), 2.58 (t, J=8.0 Hz, 2H).

### Step 3

To a solution of Pd/C (15 mg, 10% purity) in THF (10 mL) was added 8-fluoro-6-nitro-3,4-dihydro-1H-quinolin-2-one (150 mg, 713.74 μmol, 1 eq) under Ar atmosphere. The suspension was degassed and purged with H₂ for 3 times. The mixture was stirred under H₂ (15 Psi.) at 20° C. for 2 hr. LC-MS showed 8-fluoro-6-nitro-3,4-dihydro-1H-quinolin-2-one was consumed completely and several new peaks were shown on LC-MS and ~57% of desired compound was detected. The reaction mixture was filtered and concentrated under reduced pressure to give a residue. Compound 6-amino-8-fluoro-3,4-dihydro-1H-quinolin-2-one (80 mg, crude) was obtained.

### Step 4

To a solution of 6-amino-8-fluoro-3,4-dihydro-1H-quinolin-2-one (30 mg, 166.50 μmol, 1 eq) and 3-(2,2,2-trifluoroethyl)pyridine-4-carboxylic acid (52.39 mg, 183.15 μmol, 1.1 eq, HBr) in Py (1 mL) was added EDCI (47.88 mg, 249.75 μmol, 1.5 eq). The mixture was stirred at 45° C. for 2 hr. LC-MS showed 6-amino-8-fluoro-3,4-dihydro-1H-quinolin-2-one was consumed completely and several new peaks were shown on LC-MS and ~40% of desired compound was detected. The reaction mixture was concentrated under reduced pressure to give a residue. The residue was purified by prep-TLC (SiO₂, Ethyl acetate:MeOH=10:1). Compound N-(8-fluoro-2-oxo-3,4-dihydro-1H-quinolin-6-yl)-3-(2,2,2-trifluoroethyl)pyridine-4-carboxamide (12 mg, 32.67 μmol, 19.62% yield) was obtained. (M+H)⁺: 368.0. ¹H NMR (400 MHz, METHANOL-d₄) δ ppm 8.69 (t, J=4.0 Hz, 2H), 7.63 (d, J=4.0 Hz, 1H), 7.54 (d, J=8.0 Hz, 1H), 7.27 (s, 1H), 3.80-3.90 (m, 2H), 3.02 (t, J=8.0 Hz, 2H), 2.62 (t, J=8.0 Hz, 2H).

### Example 12: Synthesis of N-(8-cyano-2-oxo-1,2,3,4-tetrahydroquinolin-6-yl)-3-(2,2,2-trifluoroethyl)isonicotinamide (Compound 16)

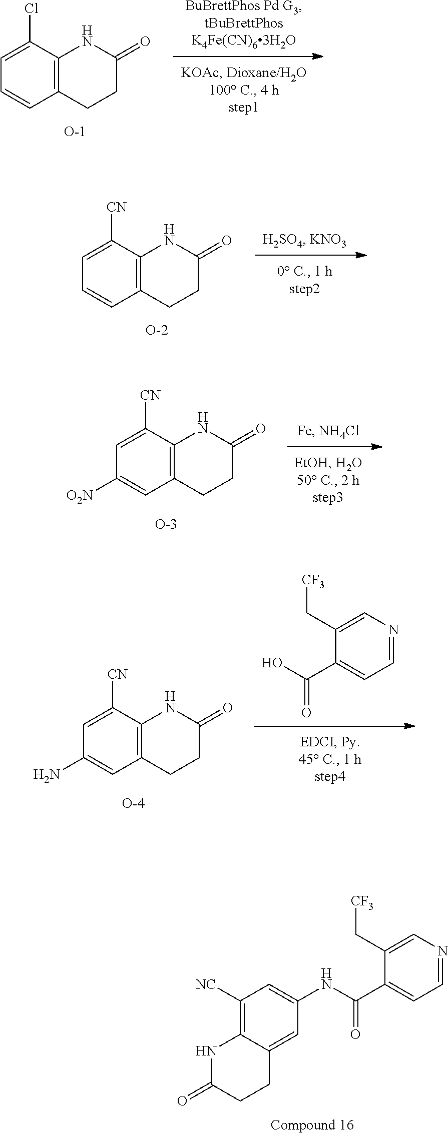

### Step 1:

To a solution of 8-chloro-3,4-dihydro-1H-quinolin-2-one (500 mg, 2.75 mmol, 1 eq) in dioxane (4 mL) and H₂O (3 mL) was added KOAc (108.08 mg, 1.10 mmol, 0.4 eq) and BuBrettPhos Pd G3(235.22 mg, 275.30 μmol, 0.1 eq) and ditert-butyl-[3,6-dimethoxy-2-(2,4,6-triisopropylphenyl)phenyl]phosphane (133.44 mg, 275.30 μmol, 0.1 eq) and K₄[Fe(CN)₆](811.25 mg, 2.20 mmol, 0.8 eq). The mixture was stirred at 100° C. for 4 hr. TLC indicated 8-chloro-3,4-dihydro-1H-quinolin-2-one was consumed completely and one new spot formed. The reaction mixture was filtered and concentrated under reduced pressure to give a residue. The residue was purified by column chromatography (SiO₂, Petroleum ether/Ethyl acetate=10/1 to 3/1). Compound 2-oxo-3,4-dihydro-1H-quinoline-8-carbonitrile (300 mg, 1.74 mmol, 63.29% yield) was obtained.

### Step 2

To a 100 mL three neck bottle was added the solution of 2-oxo-3,4-dihydro-1H-quinoline-8-carbonitrile (300 mg, 1.74 mmol, 1 eq) in H₂SO₄ (3 mL). Then to the mixture was added KNO₃ (30 mg, 296.73 μmol, 0.17 eq) in portions at 0° C. The mixture was stirred at 0° C. for 1 h. TLC indicated 2-oxo-3,4-dihydro-1H-quinoline-8-carbonitrile was consumed completely and one new spot formed. The mixture was poured into ice (10 mL) slowly. The reaction mixture was diluted with H₂O (10 mL) and extracted with EtOAc (5 mL*3). The combined organic layers were washed with brine (5 mL*3), dried over Na₂SO₄, filtered and concentrated under reduced pressure to give a residue. The crude product was triturated with (Petroleum ether:Ethyl acetate=10:1) 5 mL at 20° C. for 20 min. Compound 6-nitro-2-oxo-3,4-dihydro-1H-quinoline-8-carbonitrile (200 mg, 920.89 μmol, 52.85% yield) was obtained.

### Step 3

To a solution of 6-nitro-2-oxo-3,4-dihydro-1H-quinoline-8-carbonitrile (200 mg, 920.89 μmol, 1 eq) in EtOH (2 mL) and H₂O (2 mL) was added Fe (257.14 mg, 4.60 mmol, 5 eq) and NH₄Cl (492.60 mg, 9.21 mmol, 10 eq). The mixture was stirred at 50° C. for 2 hr. TLC indicated 6-nitro-2-oxo-3,4-dihydro-1H-quinoline-8-carbonitrile was consumed completely and two new spots formed. The reaction mixture was filtered and concentrated under reduced pressure to give a residue. The residue was purified by prep-TLC (SiO₂, Petroleum ether/Ethyl acetate=0/1). Compound 6-amino-2-oxo-3,4-dihydro-1H-quinoline-8-carbonitrile (100 mg, 534.19 umol, 58.01% yield) was obtained.

### Step 4

To a solution of 6-amino-2-oxo-3,4-dihydro-1H-quinoline-8-carbonitrile (50 mg, 267.10 μmol, 1 eq) and 3-(2,2,2-trifluoroethyl)pyridine-4-carboxylic acid (76.40 mg, 267.10 μmol, 1 eq, HBr) in Py (1 mL) was added EDCI (76.80 mg, 400.65 μmol, 1.5 eq). The mixture was stirred at 45° C. for 1 hr. TLC indicated 6-amino-2-oxo-3,4-dihydro-1H-quinoline-8-carbonitrile was consumed completely and one new spot formed. The reaction mixture was concentrated under reduced pressure to give a residue. The residue was purified by prep-TLC (SiO₂, Ethyl acetate/MeOH=20/1). Compound N-(8-cyano-2-oxo-3,4-dihydro-1H-quinolin-6-yl)-3-(2,2,2-trifluoroethyl)pyridine-4-carboxamide (20 mg, 50.64 μmol, 18.96% yield) was obtained. (M+H)+: 375.0. ¹H NMR (400 MHz, METHANOL-d4) δ ppm 8.65-8.72 (m, 2H), 7.87 (d, J=2.4 Hz, 1H), 7.74 (s, 1H), 7.65 (d, J=4.8 Hz, 1H), 3.96 (q, J=10.8 Hz, 2H), 3.03 (t, J=7.6 Hz, 2 H), 2.64 (t, J=7.6 Hz, 2H).

### Example 13: Synthesis of 3-chloro-5-ethyl-N-(8-methyl-2-oxo-1,2,3,4-tetrahydroquinolin-6-yl)isonicotinamide (Compound 7)

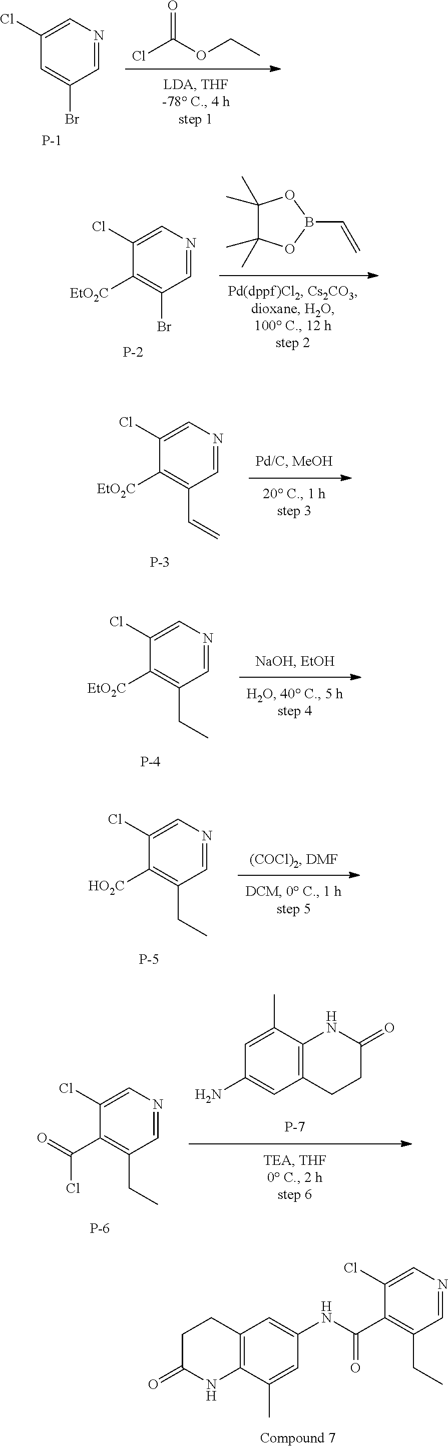

### Step 1

To the mixture of 3-bromo-5-chloro-pyridine (5 g, 25.98 mmol, 1 eq) in THF (200 mL) at 500 mL three neck bottle was added LDA (2 M, 25.98 mL, 2 eq) drop-wise at −78° C. under N₂. The reaction mixture was stirred at −78° C. for 1 h. To the mixture was added ethyl carbonochloridate (5.63 g, 51.88 mmol, 4.94 mL, 2.00 eq) in THF (100 mL) drop-wise slowly over 1 h. The final mixture was stirred at −78° C. for 2 h. LC-MS showed 3-bromo-5-chloro-pyridine was consumed completely. The reaction mixture was quenched by pour into 200 mL sat. NH₄Cl at 0° C. and diluted with H₂O 50 mL and extracted with EtOAc (80 mL*3). The combined organic layers were washed with brine (50 mL*3), dried over Na₂SO₄, filtered and concentrated under reduced pressure to give a residue. The residue was purified by column chromatography (SiO₂, Petroleum ether/Ethyl acetate=50/1 to 10/1). Compound ethyl 3-bromo-5-chloro-pyridine-4-carboxylate (3.5 g, 13.23 mmol, 50.93% yield) was obtained. (M+H)⁺: 264.0, 266.0. ¹H NMR (400 MHz, DMSO-d₆) δ ppm 8.87 (d, J=3.2 Hz, 1H), 8.81 (d, J=2.4 Hz, 1H), 4.20-4.25 (m, 2H), 1.34 (t, J=6.8 Hz, 3H)

### Step 2

To the mixture of ethyl 3-bromo-5-chloro-pyridine-4-carboxylate (2 g, 7.56 mmol, 1 eq) and 4,4,5,5-tetramethyl-2-vinyl-1,3,2-dioxaborolane (1.16 g, 7.56 mmol, 1.28 mL, 1 eq) in dioxane (20 mL) and H₂O (2 mL) was added Cs₂CO₃ (7.39 g, 22.68 mmol, 3 eq) and Pd(dppf)Cl₂ (553.27 mg, 756.13 μmol, 0.1 eq). Then the reaction mixture was stirred at 100° C. for 12 h. TLC indicated ethyl 3-bromo-5-chloro-pyridine-4-carboxylate was consumed completely and one new spot formed. The reaction mixture was diluted with H₂O 200 mL and extracted with EtOAc (50 mL*3). The combined organic layers were washed with brine (20 mL*3), dried over Na₂SO₄, filtered and concentrated under reduced pressure to give a residue. The residue was purified by column chromatography (SiO₂, Petroleum ether/Ethyl acetate=100/1 to 10/1). Compound ethyl 3-chloro-5-vinyl-pyridine-4-carboxylate (1.3 g, 6.14 mmol, 81.23% yield) was obtained.

### Step 3

To a solution of ethyl 3-chloro-5-vinyl-pyridine-4-carboxylate (1.2 g, 5.67 mmol, 1 eq) in MeOH (3 mL) was added Pd/C (100 mg, 10% purity) under N₂ atmosphere. The suspension was degassed and purged with H₂ for 3 times. The mixture was stirred under H₂ (15 Psi) at 20° C. for 1 h. The reaction mixture was filtered and filter liquor was concentrated under reduced pressure to give a residue. Compound ethyl 3-chloro-5-ethyl-pyridine-4-carboxylate (1.1 g, 5.15 mmol, 90.80% yield) was obtained.

### Step 4

To the mixture of ethyl 3-chloro-5-ethyl-pyridine-4-carboxylate (400 mg, 1.87 mmol, 1 eq) in H₂O (3 mL) and EtOH (6 mL) was added NaOH (149.76 mg, 3.74 mmol, 2 eq). The reaction mixture was stirred at 40° C. for 5 h. LC-MS showed ethyl 3-chloro-5-ethyl-pyridine-4-carboxylate was consumed completely and desired mass was detected. The reaction mixture was filtered and filter liquor was concentrated under reduced pressure to give a residue. The residue was purified by prep-HPLC (column: Phenomenex luna C18 250*50 mm*10 μm; mobile phase: [water (HCl)-ACN]; B %: 1%-20%, 10 min). Compound 3-chloro-5-ethyl-pyridine-4-carboxylic acid (130 mg, 585.41 umol, 31.27% yield, HCl) was obtained. (M+H)⁺: 196.1. ¹H NMR (400 MHz, DMSO-d₆) δ ppm 8.58 (s, 1H), 8.54 (s, 1H), 2.63 (q, J=7.6 Hz, 2H), 1.19 (t, J=7.6 Hz, 3H)

### Step 5

To the mixture of 3-chloro-5-ethyl-pyridine-4-carboxylic acid (40 mg, 180.12 umol, 1 eq, HCl salt) in DCM (5 mL) was added oxalyl dichloride (68.59 mg, 540.37 μmol, 47.30 uL, 3 eq) drop-wise at 0° C. Then the reaction mixture was stirred at 0° C. for 1 h. The reaction mixture was concentrated under reduced pressure to give a residue. The crude product 3-chloro-5-ethyl-pyridine-4-carbonyl chloride (30 mg, crude) was used into the next step without further purification.

### Step 6

To the mixture of 6-amino-8-methyl-3,4-dihydro-1H-quinolin-2-one (25.91 mg, 147.02 μmol, 1 eq), TEA (44.63 mg, 441.06 μmol, 61.39 μL, 3 eq) in THF (5 mL) was added 3-chloro-5-ethyl-pyridine-4-carbonyl chloride (30 mg, 147.02 μmol, 1 eq) in DCM (2 mL) drop-wise at 0° C. Then the reaction mixture was stirred at 0° C. for 2 h. LC-MS showed 3-chloro-5-ethyl-pyridine-4-carbonyl chloride was consumed completely and one main peak with desired m/z was detected. The reaction mixture was diluted with H₂O 10 mL and extracted with EtOAc (5 mL*3). The combined organic layers were washed with brine (3 mL*3), dried over Na₂SO₄, filtered and concentrated under reduced pressure to give a residue. The residue was purified by prep-TLC (SiO₂, Petroleum ether:Ethyl acetate=0:1). Compound 3-chloro-5-ethyl-N-(8-methyl-2-oxo-3,4-dihydro-1H-quinolin-6-yl)pyridine-4-carboxamide (25 mg, 70.53 μmol, 47.98% yield) was obtained. (M+H)⁺: 344.2. ¹H NMR (400 MHz, DMSO-d₆) δ ppm 10.57 (s, 1H), 9.45 (s, 1H), 8.59 (s, 1H), 8.55 (s, 1H), 7.25-7.37 (m, 2H), 2.85 (t, J=7.6 Hz, 2H), 2.63-2.68 (m, 2H), 2.43 (t, J=7.2 Hz, 2H), 2.21 (s, 3H), 1.20 (t, J=7.2 Hz, 3H).

### Example 14: Synthesis of 2-cyano-N-(8-methyl-2-oxo-1,2,3,4-tetrahydroquinolin-6-yl)-3-(2,2,2-trifluoroethyl)isonicotinamide (Compound 9)

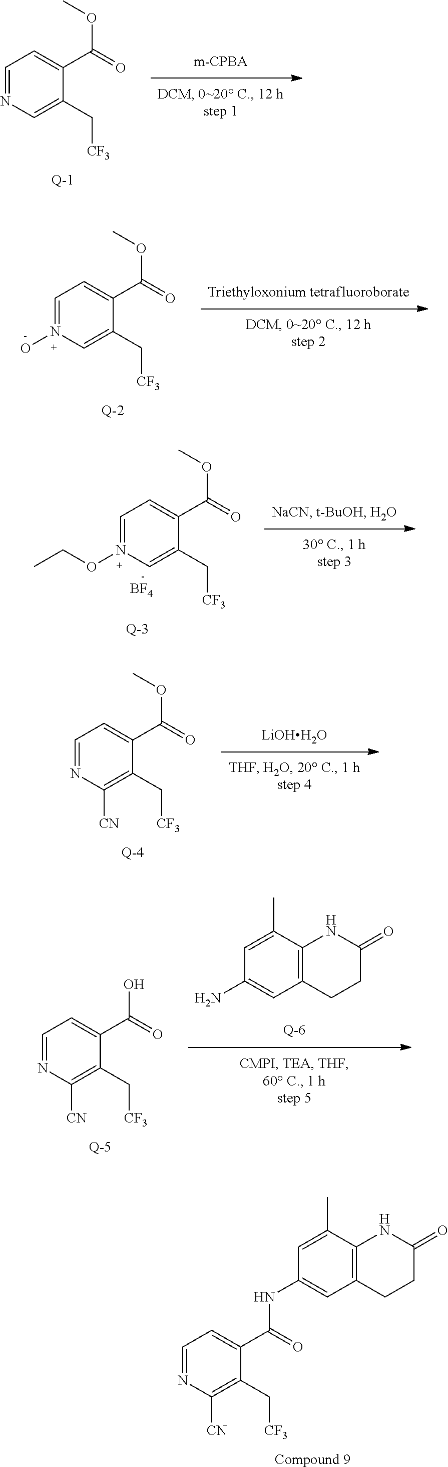

### Step 1

To the mixture of methyl 3-(2,2,2-trifluoroethyl)pyridine-4-carboxylate (5 g, 22.8 mmol, 1 eq) in DCM (75 mL) was added m-CPBA (6.02 g, 29.6 mmol, 85% purity, 1.3 eq) in portions at 0° C. Then the mixture was stirred at 20° C. for 12 h. To the mixture was added sat. Na₂SO₃ (200 ml) and the mixture was stirred at 20° C. for 30 min. The mixture was extracted with DCM (70 mL*3). The combined organic phase was washed with brine (80 mL), dried with anhydrous Na₂SO₄, filtered and concentrated in vacuum. The residue was purified by column chromatography (SiO₂, Petroleum ether/Ethyl acetate=3/1 to 2/1). Compound methyl 1-oxido-3-(2, 2, 2-trifluoroethyl)pyridin-1-ium-4-carboxylate (4.8 g, 20.4 mmol, 89.4% yield) was obtained. (M+H)⁺: 236.1.

### Step 2

To a solution of methyl 1-oxido-3-(2,2,2-trifluoroethyl)pyridin-1-ium-4-carboxylate (2.5 g, 10.6 mmol, 1 eq) in DCM (30 mL) was added Triethyloxonium tetrafluoroborate (6.06 g, 31.8 mmol, 4.56 mL, 3 eq) at 0° C. The mixture was stirred at 20° C. for 12 h. LCMS showed ~47% of desired compound was detected. The reaction mixture was diluted with H₂O 30 mL and extracted with DCM 10 mL*3. The combined organic layers were washed with brine 10 mL*3, dried over Na₂SO₄, filtered and concentrated under reduced pressure to give a residue. The residue was purified by column chromatography (SiO₂, Petroleum ether/Ethyl acetate=5/1 to 3/1). Compound methyl 1-ethoxy-3-(2,2,2-trifluoroethyl)pyridin-1-ium-4-carboxylate (2.4 g, 6.84 mmol, 64.3% yield, [BF₄]⁻¹) was obtained. (M+H)⁺: 265.1.

### Step 3

To a solution of methyl 1-ethoxy-3-(2,2,2-trifluoroethyl)pyridin-1-ium-4-carboxylate (3 g, 9.04 mmol, 1 eq, [BF₄]⁻¹) in H₂O (60 mL) and t-BuOH (30 mL) was added NaCN (640 mg, 13.0 mmol, 1.45 eq) in H₂O (15 mL) at 30° C. The mixture was stirred at 30° C. for 1 h. LC-MS showed methyl 1-ethoxy-3-(2,2,2-trifluoroethyl)pyridin-1-ium-4-carboxylate was consumed completely and one main peak with desired was detected. The reaction mixture was cooled to 20° C. Then the mixture was adjusted to pH=11 by sat. Na₂CO₃. The aqueous phase was extracted with ethyl acetate (40 mL*3). The combined organic phase was washed with brine (30 mL), dried with anhydrous Na₂SO₄, filtered and concentrated in vacuum. The residue was purified by column chromatography (SiO₂, Petroleum ether/Ethyl acetate=3/1 to 2/1). Compound methyl 2-cyano-3-(2,2,2-trifluoroethyl)pyridine-4-carboxylate (460 mg, 1.88 mmol, 16.4% yield) was obtained. (M+H)+: 245.1. ¹H NMR (400 MHz, CHLOROFORM-d) δ ppm 8.77 (d, J=5.2 Hz, 1H), 7.91 (d, J=5.2 Hz, 1H), 4.23-4.28 (m, 2H), 3.93 (s, 3H).

### Step 4

To a solution of ethyl 2-cyano-3-(2,2,2-trifluoroethyl)pyridine-4-carboxylate (100 mg, 409 μmol, 1 eq) in THF (3 mL) and H₂O (3 mL) was added LiOH·H₂O (19.6 mg, 819, μmol, 2 eq). The mixture was stirred at 20° C. for 1 h under N₂ atmosphere. LCMS showed methyl 2-cyano-3-(2,2,2-trifluoroethyl)pyridine-4-carboxylate was consumed completely and one main peak with desired m/z was detected. The reaction was concentrated in vacuum to remove THF. Then the mixture was adjusted to pH=2~3 by 1N HCl. Then the mixture was filtered. The filter cake was concentrated in vacuum. Compound 2-cyano-3-(2,2,2-trifluoroethyl)pyridine-4-carboxylic acid (50 mg, 217 μmol, 53.05% yield) was obtained. (M+H)+: 231.1. ¹H NMR (400 MHz, METHANOL-d₄) δ ppm 8.65 (d, J=4.8 Hz, 1H), 7.79 (d, J=5.2 Hz, 1H), 4.27-4.35 (m, 2H).

### Step 5

A mixture of 2-cyano-3-(2,2,2-trifluoroethyl)pyridine-4-carboxylic acid (80 mg, 300 μmol, 1 eq, HCl), 6-amino-8-methyl-3,4-dihydro-1H-quinolin-2-one (47.5 mg, 270 μmol, 0.9 eq), CMPI (114 mg, 450 1 μmol, 1.5 eq) and TEA (91.0 mg, 900 μmol, 125 μL, 3 eq) in THF (5 mL) was degassed and purged with N₂ for 3 times, and then the mixture was stirred at 60° C. for 1 h under N₂ atmosphere. The reaction mixture was concentrated under reduced pressure. The residue was purified by prep-TLC (SiO₂, Petroleum ether/Ethyl acetate=0/1). Compound 2-cyano-N-(8-methyl-2-oxo-3,4-dihydro-1H-quinolin-6-yl)-3-(2,2,2-trifluoroethyl)pyridine-4-carboxamide (12 mg, 29.76 μmol, 9.92% yield) was obtained. (M+H)⁺: 389.1. ¹H NMR (400 MHz, DMSO-d₆) δ ppm 10.71 (s, 1H), 9.46 (s, 1H), 8.94 (d, J=4.8 Hz, 1H), 7.99 (d, J=4.8 Hz, 1H), 7.37 (s, 1H), 7.20 (s, 1H), 4.16-4.24 (m, 2H), 2.86 (t, J=7.2 Hz, 2H), 2.43 (t, J=8.0 Hz, 2H), 2.21 (s, 3H).

### Example 15: Synthesis of 2-cyano-N-(8-methyl-2-oxo-1,2,3,4-tetrahydroquinolin-6-yl)-5-(2,2,2-trifluoroethyl)isonicotinamide (Compound 10)

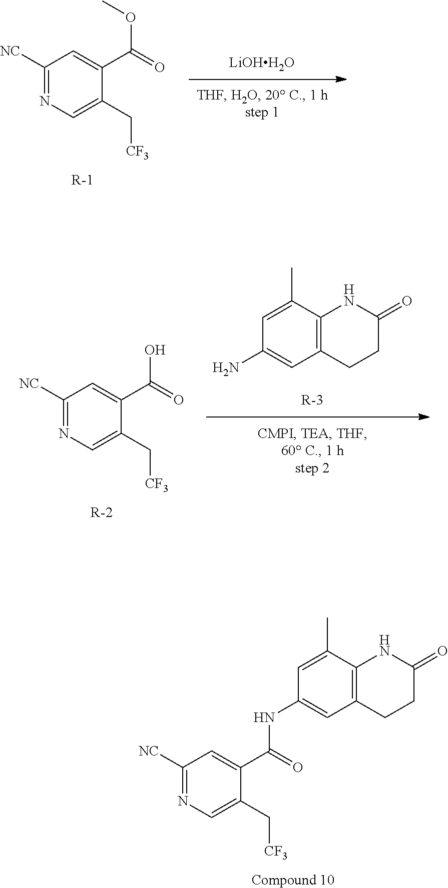

### Step 1

To a solution of methyl 2-cyano-5-(2,2,2-trifluoroethyl)pyridine-4-carboxylate (100 mg, 409 μmol, 1 eq) in THF (3 mL) and H₂O (3 mL) was added LiOH·H₂O (19.6 mg, 819 μmol, 2 eq). The mixture was stirred at 20° C. for 1 h under N₂ atmosphere. LCMS showed methyl 2-cyano-3-(2,2,2-trifluoroethyl)pyridine-4-carboxylate was consumed completely and one main peak with desired m/z was detected. The reaction was concentrated in vacuum to remove THF. Then the mixture was adjusted to pH=2~3 by 1N HCl. Then the mixture was filtered. The filter cake was concentrated in vacuum. Compound 2-cyano-5-(2,2,2-trifluoroethyl)pyridine-4-carboxylic acid (80 mg, 782 μmol, 63.6% yield) was obtained. (M+H)⁺: 231.1. ¹H NMR (400 MHz, METHANOL-d₄) δ ppm 8.76 (s, 1H), 8.19 (s, 1H), 4.16-4.24 (m, 2H).

### Step 2

A mixture of 2-cyano-5-(2,2,2-trifluoroethyl)pyridine-4-carboxylic acid (40 mg, 150 μmol, 1 eq, HCl), 6-amino-8-methyl-3,4-dihydro-1H-quinolin-2-one (23.7 mg, 135 μmol, 0.9 eq), CMPI (57.5 mg, 225 μmol, 1.5 eq) and TEA (45.5 mg, 450 μmol, 62.6 μL, 3 eq) in THF (5 mL) was degassed and purged with N₂ for 3 times, and then the mixture was stirred at 60° C. for 1 h under N₂ atmosphere. LCMS showed no 2-cyano-5-(2,2,2-trifluoroethyl)pyridine-4-carboxylic acid remained. Several new peaks were shown on LCMS and ~43% of desired compound was detected. The reaction mixture was concentrated under reduced pressure. The residue was purified by prep-TLC (SiO₂, Petroleum ether/Ethyl acetate=0/1). Compound 2-cyano-N-(8-methyl-2-oxo-3,4-dihydro-1H-quinolin-6-yl)-5-(2,2,2-trifluoroethyl)pyridine-4-carboxamide (10 mg, 25.5 μmol, 17.0% yield) was obtained. (M+H)+: 389.1. ¹H NMR (400 MHz, DMSO-d6) δ ppm 10.61 (s, 1H), 9.47 (s, 1H), 8.92 (s, 1H), 8.35 (s, 1H), 7.38 (s, 1H), 7.31 (s, 1H), 4.06-4.15 (m, 2H), 2.86 (t, J=7.6 Hz, 2H), 2.45 (t, J=2.8 Hz, 2H), 2.21 (s, 3H).

### Example 16: Synthesis of N-[8-(4-fluoropyrazol-1-yl)-2-oxo-3,4-dihydro-1H-quinolin-6-yl]-3-(2,2,2-trifluoroethyl)pyridine-4-carboxamide (Compound 22)

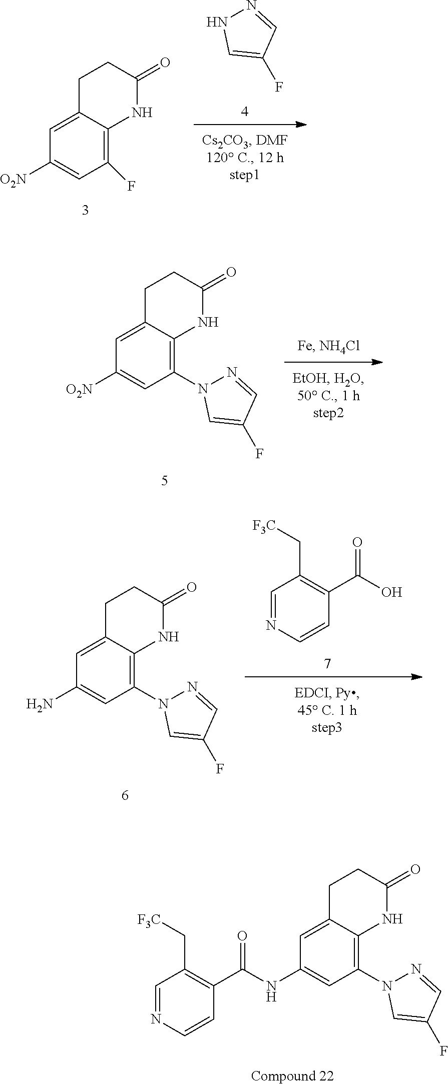

### Step 1

To a solution of 8-fluoro-6-nitro-3,4-dihydro-1H-quinolin-2-one (200 mg, 952 μmol, 1 eq) and 4-fluoro-1H-pyrazole (123 mg, 1.43 mmol, 1.5 eq) in DMF (2 mL) was added Cs₂CO₃ (620 mg, 1.90 mmol, 2 eq). The mixture was stirred at 120° C. for 12 hr. The reaction mixture was poured into H₂O (5 mL) slowly, filtered and the filter cake concentrated under reduced pressure to give a residue. Compound 8-(4-fluoropyrazol-1-yl)-6-nitro-3,4-dihydro-1H-quinolin-2-one (80 mg, 278 μmol, 29.2% yield, 96% purity) was obtained as a yellow solid. [M+H]⁺: 277.0. ¹H NMR (400 MHz, DMSO-d₆) δ ppm 10.42 (s, 1H), 8.77 (d, J=4.0 Hz, 1H), 8.20-8.30 (m, 2H), 8.06 (d, J=4.0 Hz, 1H), 3.15 (t, J=7.2 Hz, 2H), 2.57-2.64 (m, 2H).

### Step 2

To a solution of 8-(4-fluoropyrazol-1-yl)-6-nitro-3,4-dihydro-1H-quinolin-2-one (70 mg, 253 μmol, 1 eq) in EtOH (8 mL) and H₂O (4 mL) was added Fe (70.8 mg, 1.27 mmol, 5 eq) and NH₄Cl (135 mg, 2.53 mmol, 10 eq). The mixture was stirred at 50° C. for 1 hr. The crude was combined another crude for workup (total 80 mg). The reaction mixture was filtered and then diluted with H₂O 5 mL and extracted with EtOAc 30 mL (10 mL*3). The combined organic layers were washed with brine 20 mL (10 mL*2), dried over anhydrous Na₂SO₄, filtered and concentrated under reduced pressure to give a residue. Compound 6-amino-8-(4-fluoropyrazol-1-yl)-3,4-dihydro-1H-quinolin-2-one (50 mg, 70% yield) was obtained as a yellow solid.

### Step 3

To a solution of 6-amino-8-(4-fluoropyrazol-1-yl)-3,4-dihydro-1H-quinolin-2-one (40 mg, 162 μmol, 1 eq) and 3-(2,2,2-trifluoroethyl)pyridine-4-carboxylic acid (47.1 mg, 195 μmol, 1.2 eq, HCl) in Py (2 mL) was added EDCI (46.7 mg, 244 μmol, 1.5 eq). The mixture was stirred at 45° C. for 1 hr. The reaction mixture was concentrated under reduced pressure to give a residue. The residue was combined to another crude for further purification (total 50 mg). The residue was purified by prep-HPLC (column: Phenomenex luna C18 100*40 mm*5 μm; mobile phase: [H₂O (0.04% HCl)-ACN]; gradient: 20%-50% B over 8.0 min). Compound N-[8-(4-fluoropyrazol-1-yl)-2-oxo-3,4-dihydro-1H-quinolin-6-yl]-3-(2,2,2-trifluoroethyl)pyridine-4-carboxamide (100% purity, HCl) was (45 mg, 47% yield) was obtained as a yellow solid. [M+H]⁺: 434.1. ¹H NMR (400 MHz, METHANOL-d₄) δ ppm 8.93-9.03 (m, 2H), 8.18 (d, J=4.4 Hz, 1H), 8.10-8.15 (m, 1H), 7.77-7.83 (m, 2H), 7.52-7.57 (m, 1H), 4.04-4.17 (m, 2H), 3.10 (t, J=7.2 Hz, 2H), 2.61-2.69 (m, 2H).

### Example 17: Synthesis of N-[2-oxo-8-[4-(trifluoromethyl) pyrazol-1-yl]-3,4-dihydro-1H-quinolin-6-yl]-3-(2,2,2-trifluoroethyl)pyridine-4-carboxamide (Compound 23)

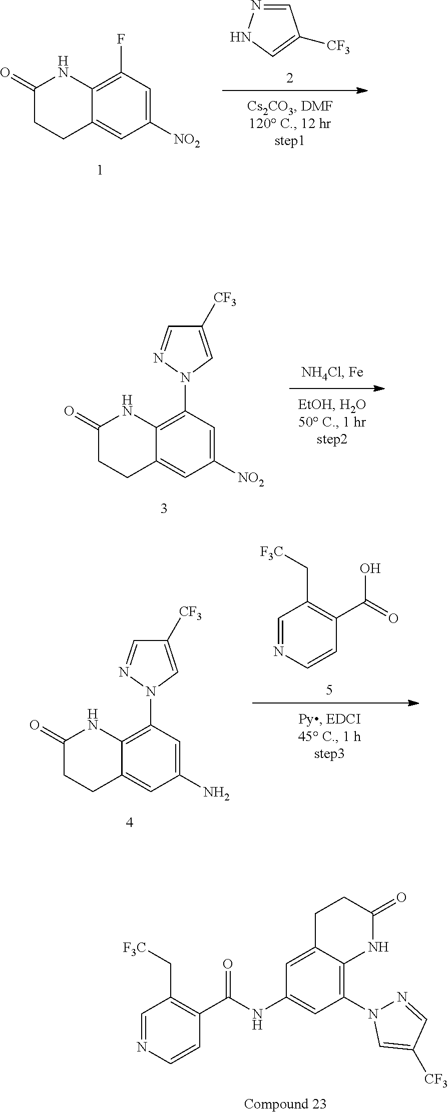

### Step 1

To a solution of 8-fluoro-6-nitro-3,4-dihydro-1H-quinolin-2-one (0.2 g, 951 μmol, 1 eq) and 4-(trifluoromethyl)-1H-pyrazole (155 mg, 1.14 mmol, 1.2 eq) in DMF (2 mL) was added Cs₂CO₃ (620 mg, 1.90 mmol, 2 eq) The mixture was stirred at 120° C. for 12 hr. The reaction was messy according to TLC. The reaction mixture was quenched by addition H₂O 5 mL at 0° C., and extracted with EtOAc (5 mL*3). The combined organic layers were washed with brine (5 mL*2), dried over Na₂CO₃, filtered and concentrated under reduced pressure to give a residue. The residue was purified by column chromatography (SiO₂, Petroleum ether/Ethyl acetate=100/0 to 90/10) to give cnitro-8-[4-(trifluoromethyl) pyrazol-1-yl]-3,4-dihydro-1H-quinolin-2-one (0.15 g, 285 mol, 29.9% yield, 62% purity) was obtained as a white solid. ¹H NMR (400 MHz, DMSO-d₆) δ ppm 10.25 (s, 1H), 9.11 (s, 1H), 8.36 (d, J=4.8 Hz, 1H), 7.98-8.11 (m, 1H), 7.91 (s, 1H), 3.12-3.22 (m, 2H), 2.57-2.65 (m, 2H)

### Step 2

To a solution of 6-nitro-8-[4-(trifluoromethyl) pyrazol-1-yl]-3,4-dihydro-1H-quinolin-2-one (0.1 g, 306 mol, 1 eq) in ETOH (1 mL) and H₂O (0.2 mL) was added NH₄Cl (163 g, 3.07 mmol, 10 eq) and Fe (85.6 mg, 1.53 mmol, 5 eq). The mixture was stirred at 50° C. for 1 hr. The reaction mixture was filtered and concentrated under reduced pressure to give a residue. The mixture was poured water (3 mL) and extracted with EtOAc (5 mL*2). The combined organic layers were washed with brine (5 mL*2), dried over Na₂SO₄, filtered and concentrated under reduced pressure to give compound 6-amino-8-[4-(trifluoromethyl) pyrazol-1-yl]-3,4-dihydro-1H-quinolin-2-one (0.11 g crude) was obtained as a white solid.

### Step 3

To a solution of 3-(2,2,2-trifluoroethyl)pyridine-4-carboxylic acid (69.2 mg, 337 μmol, 1 eq) and 6-amino-8-[4-(trifluoromethyl) pyrazol-1-yl]-3,4-dihydro-1H-quinolin-2-one (0.1 g, 337 μmol, 1 eq) in py. (1 mL) was added EDCI (129 mg, 675 μmol, 2 eq). The mixture was stirred at 45° C. for 1 hr. LCMS showed 6-amino-8-[4-(trifluoromethyl) pyrazol-1-yl]-3,4-dihydro-1H-quinolin-2-one was consumed completely and one main peak with desired 484.1 m/z was detected. The reaction mixture was concentrated under reduced pressure to give a residue. The residue was purified by prep-HPLC (column: Phenomenex luna C18 100*40 mm*5 μm; mobile phase: [H₂O (0.04% HCl)-ACN]; gradient: 25%-55% B over 8.0 min) to give compound N-[2-oxo-8-[4-(trifluoromethyl) pyrazol-1-yl]-3,4-dihydro-1H-quinolin-6-yl]-3-(2,2,2-trifluoroethyl)pyridine-4-carboxamide (99.4% purity, HCl, 33 mg) was obtained as a yellow solid. (M+H)⁺: 484.0 ¹H NMR (400 MHz, DMSO-d₆) δ ppm 10.82 (s, 1H), 9.61 (s, 1H), 8.86 (s, 1H), 8.78 (d, J=4.8 Hz, 1H), 8.75 (s, 1H), 8.29 (s, 1H), 7.77 (d, J=2.4 Hz, 1H), 7.63 (s, 1H), 7.65 (d, J=9.2 Hz, 1H), 4.01 (q, J=11.2 Hz, 2H), 3.00-3.05 (m, 2H), 2.53 (s, 2H).

### Example 18: Synthesis of N-[8-(4-cyanopyrazol-1-yl)-2-oxo-3,4-dihydro-1H-quinolin-6-yl]-3-(2,2,2-trifluoroethyl)pyridine-4-carboxamide (Compound 24)

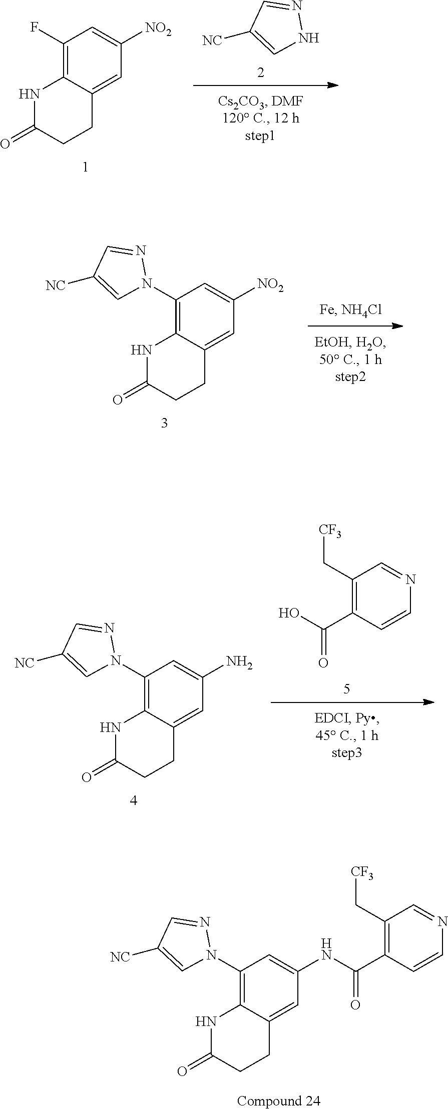

### Step 1

To a solution of 1H-pyrazole-4-carbonitrile (332 mg, 3.57 mmol, 1.5 eq) and 8-fluoro-6-nitro-3,4-dihydro-1H-quinolin-2-one (500 mg, 2.38 mmol, 1 eq) in DMF (5 mL) was added Cs₂CO₃ (1.55 g, 4.76 mmol, 2 eq). The mixture was stirred at 120° C. for 12 hr. The reaction mixture was poured into H₂O (5 mL) slowly, filtered and the filter cake concentrated under reduced pressure to give a residue. Compound 1-(6-nitro-2-oxo-3,4-dihydro-1H-quinolin-8-yl) pyrazole-4-carbonitrile (300 mg, 1.04 mmol, 43.6% yield, 98% purity) was obtained as a white solid. [M+H]:⁺284.1. ¹H NMR (400 MHz, DMSO-d₆) δ ppm 10.08-10.21 (m, 1H), 9.16 (s, 1H), 8.46 (s, 1H), 8.25-8.35 (m, 2H), 3.16 (t, J=7.2 Hz, 2H), 2.60 (t, J=7.6 Hz, 2H).

### Step 2

To a solution of 1-(6-nitro-2-oxo-3,4-dihydro-1H-quinolin-8-yl) pyrazole-4-carbonitrile (100 mg, 353 μmol, 1 eq) in EtOH (4 mL) and H₂O (2 mL) was added Fe (98.6 mg, 1.77 mmol, 5 eq) and NH₄Cl (189 mg, 3.53 mmol, 10 eq). The mixture was stirred at 50° C. for 1 hr. The reaction mixture was filtered and then diluted with H₂O 5 mL and extracted with EtOAc 30 mL (10 mL*3). The combined organic layers were washed with brine 10 mL, dried over anhydrous Na₂SO₄, filtered and concentrated under reduced pressure to give a residue. Without purification. Compound 1-(6-amino-2-oxo-3,4-dihydro-1H-quinolin-8-yl) pyrazole-4-carbonitrile (80 mg, 316 μmol, 89.5% yield) was obtained as a yellow solid. [M+H]⁺: 254.0.

### Step 3:

To a solution of 1-(6-amino-2-oxo-3,4-dihydro-1H-quinolin-8-yl) pyrazole-4-carbonitrile (70 mg, 276 μmol, 1 eq) and 3-(2,2,2-trifluoroethyl)pyridine-4-carboxylic acid (80.1 mg, 331 μmol, 1.2 eq, HCl) in Py (1 mL) was added EDCI (79.5 mg, 415 μmol, 1.5 eq). The mixture was stirred at 45° C. for 1 hr. The reaction mixture was concentrated under reduced pressure to give a residue. The residue was purified by prep-HPLC (column: WePure Biotech XPt C_{18150*40*7} μm; mobile phase: [H₂O (10 mM NH₄HCO₃)-ACN]; gradient: 10%-60% B over 8.0 min). Compound N-[8-(4-cyanopyrazol-1-yl)-2-oxo-3,4-dihydro-1H-quinolin-6-yl]-3-(2,2,2-trifluoroethyl)pyridine-4-carboxamide (65 mg, 144 μmol, 52.28% yield, 97.9% purity) was obtained as a yellow solid, [M+H]⁺: 441.1. ¹H NMR (400 MHz, DMSO-d₆) δ ppm 10.83 (s, 1H), 9.57 (s, 1H), 8.99 (s, 1H), 8.67-8.82 (m, 2H), 8.40 (s, 1H), 7.58-7.76 (m, 3H), 3.90-4.05 (m, 2H), 3.01 (J=7.2 Hz, 2H), 2.51-2.55 (m, 2H).

### Example 19: Synthesis of N-(8-chloro-2-oxo-3,4-dihydro-1H-quinolin-6-yl)-2-methyl-5-(2,2,2-trifluoroethyl)pyridine-4-carboxamide (Compound 25)

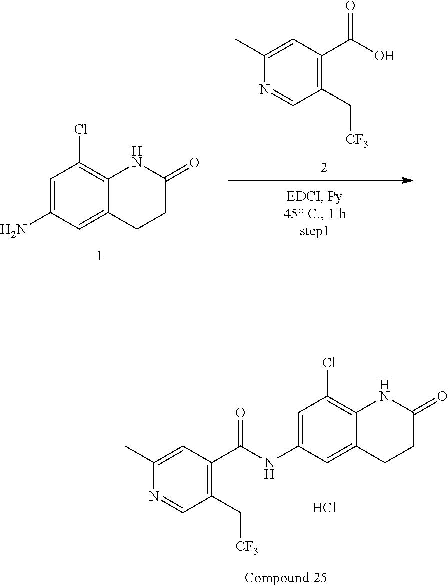

### Step 1

To a solution of 6-amino-8-chloro-3,4-dihydro-1H-quinolin-2-one (30 mg, 153 μmol, 1 eq) and 2-methyl-5-(2,2,2-trifluoroethyl)pyridine-4-carboxylic acid (46.8 mg, 183 μmol, 1.2 eq, HCl) in Py (1 mL) was added EDCI (43.8 mg, 229 μmol, 1.5 eq). The mixture was stirred at 45° C. for 1 hr. The reaction mixture was concentrated under reduced pressure to give a residue. The residue was purified by prep-HPLC (column: Phenomenex luna C18 100*40 mm*5 um; mobile phase: [H₂O (0.04% HCl)-ACN];gradient: 15%-50% B over 8.0 min). Compound N-(8-chloro-2-oxo-3,4-dihydro-1H-quinolin-6-yl)-2-methyl-5-(2,2,2-trifluoroethyl)pyridine-4-carboxamide (30 mg, 69.09 μmol, 45.28% yield, 100% purity, HCl) was obtained as a yellow solid. (M+H)⁺: 398.1. ¹H NMR (400 MHz, METHANOL-d₄) δ ppm 8.92 (s, 1H), 8.15 (s, 1H), 7.76 (d, J=2.0 Hz, 1H), 7.46-7.51 (m, 1H), 4.02-4.13 (m, 2H), 3.03 (t, J=7.2 Hz, 2H), 2.86 (s, 3H), 2.59-2.66 (m, 2H).

### Example 20: Synthesis of 2-cyano-6-methyl-N-(8-methyl-2-oxo-3,4-dihydro-1H-quinolin-6-yl)-3-(2,2,2-trifluoroethyl)pyridine-4-carboxamide (Compound 26)

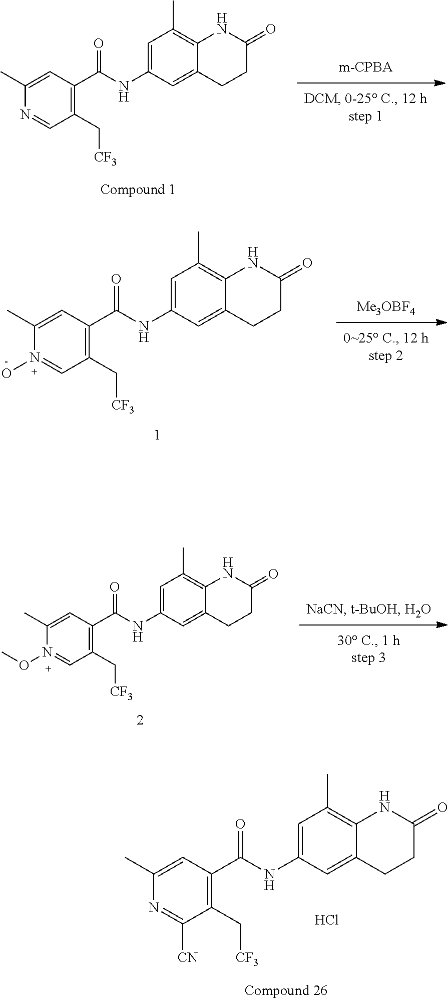

### Step 1

To a solution of 2-methyl-N-(8-methyl-2-oxo-3,4-dihydro-1H-quinolin-6-yl)-5-(2,2,2-trifluoroethyl)pyridine-4-carboxamide (1 g, 2.65 mmol, 1 eq) in DCM (20 mL) was added m-CPBA (699 mg, 3.44 mmol, 85% purity, 1.3 eq) in portions at 0° C. under N₂. The mixture was stirred at 25° C. for 12 h. To the mixture was added sat. Na₂SO₃ (50 mL) slowly at 25° C. under N₂. Then the mixture was stirred at 25° C. for 1 h. The mixture was extracted with DCM (20 mL*3). The combined organic phase was washed with brine (20 mL), dried with anhydrous Na₂SO₄, filtered and concentrated in vacuum. Compound 2-methyl-N-(8-methyl-2-oxo-3,4-dihydro-1H-quinolin-6-yl)-1-oxido-5-(2,2,2-trifluoroethyl)pyridin-1-ium-4-carboxamide (0.8 g, 2.03 mmol, 76.7% yield) was obtained as a white solid. (M+H)⁺=394.0

### Step 2

To a solution of 2-methyl-N-(8-methyl-2-oxo-3,4-dihydro-1H-quinolin-6-yl)-1-oxido-5-(2,2,2-trifluoroethyl)pyridin-1-ium-4-carboxamide (0.1 g, 254 μmol, 1 eq) in ACN (3 mL) was added trimethyloxonium;tetrafluoroborate (94.0 mg, 635 μmol, 2.5 eq) at 0° C. The mixture was stirred at 25° C. for 12 h. The reaction mixture was diluted with H₂O 20 mL and extracted with EtOAc 40 mL (20 mL*2). The combined organic layers were dried over Na₂SO₄, filtered and concentrated under reduced pressure to give a residue. Compound 1-methoxy-2-methyl-N-(8-methyl-2-oxo-3,4-dihydro-1H-quinolin-6-yl)-5-(2,2,2-trifluoroethyl)pyridin-1-ium-4-carboxamide (80 mg, crude) was obtained as a white solid. (M+H)⁺=408.1

### Step 3

To a solution of 1-methoxy-2-methyl-N-(8-methyl-2-oxo-3,4-dihydro-1H-quinolin-6-yl)-5-(2,2,2-trifluoroethyl)pyridin-1-ium-4-carboxamide (50 mg, 122.43 μmol, 1 eq) in t-BuOH (0.5 mL) and H₂O (1 mL) was added dropwise NaCN (9.00 mg, 183.65 μmol, 1.5 eq) in H₂O (0.2 mL) under N₂ at 25° C. The mixture was stirred at 30° C. for 1 h. The reaction mixture was cooled to 25° C. Then the mixture was adjusted to pH=11 by sat. Na₂CO₃. The aqueous phase was extracted with EtOAc (10 mL*3). The combined organic layers were washed with brine (10 mL*3), dried over Na²SO₄, filtered and concentrated under reduced pressure to give a residue. The residue was purified by prep-HPLC (column: Phenomenex luna C18 100*40 mm*5 μm; mobile phase: [H₂O (0.04% HCl)-ACN]; gradient: 25%-55% B over 8.0 min). Compound 2-cyano-6-methyl-N-(8-methyl-2-oxo-3,4-dihydro-1H-quinolin-6-yl)-3-(2,2,2-trifluoroethyl)pyridine-4-carboxamide (16 mg, 36.21 μmol, 29.57% yield, 99.3% purity, HCl) was obtained as a white solid. (M+H)⁺=403.1 ¹H NMR (400 MHz, DMSO-d₆) δ ppm 10.65 (s, 1H), 9.46 (s, 1H), 7.89 (s, 1H), 7.36 (s, 1H), 7.28 (d, J=1.6 Hz, 1H), 4.13 (q, J=11.2 Hz, 2H), 2.86 (t, J=6.8 Hz, 2H), 2.62 (s, 3H), 2.39-2.47 (m, 2H), 2.21 (s, 3H).

### Example 21: Aldh1a3 and Aldh1a2 Enzyme Inhibition Assays

### Aldh1a3 Enzyme Inhibition Assay

Recombinant protein extraction: pET-Aldh1a3 transformed BL21-DE3 cultures induced at 20° C. for 19 h with 0.3 mM IPTG rocking. Cultures were spun at 3500 g for 10 min, supernatants were poured off and allowed to drain fully. Cells were resuspended in 10 mM HEPES pH 7.4, 10 mM KCl. Cells were freeze-thawed in liquid nitrogen and then a 37° C. water bath for 10 cycles followed by ultrasonication at 50% amplitude, 3 sec on, 9 sec off for 10 cycles at 4° C. Cell extracts were spun at 16000×g for 5 minutes.

Reaction performed at 20° C. in reaction buffer (10 mM HEPES pH 7.4, 10 mM KCl, 0.1 M Resazurin, 1 mg/mL BSA, 200 μM NAD+, diaphorase and aldehyde substrate). Recombinant enzyme and inhibitor added immediately before assay. Reaction rate measured by resorufin fluorescence.

### Aldh1a2 Enzyme Inhibition Assay

Recombinant protein extraction: pET-Aldh1a2 transformed BL21-DE3 cultures induced at 20° C. for 19 h with 0.3 mM IPTG rocking. Cultures were spun at 3500 g for 10 min, supernatants were poured off and allowed to drain fully. Cells were resuspended in 10 mM HEPES pH 7.4, 10 mM KCl. Cells were freeze-thawed in liquid nitrogen and then a 37° C. water bath for 10 cycles followed by ultrasonication at 50% amplitude, 3 sec on, 9 sec off for 10 cycles at 4° C. Cell extracts were spun at 16000×g for 5 minutes.

Reaction performed at 20° C. in reaction buffer (10 mM HEPES pH 7.4, 10 mM KCl, 0.1 M Resazurin, 1 mg/mL BSA, 200 μM NAD+, diaphorase and aldehyde substrate). Recombinant enzyme and inhibitor added immediately before assay. Reaction rate measured by resorufin fluorescence.

The IC₅₀ values of selected tested compounds are shown in Table 2 below. The hAldh1a3:hAldh1a2 ratio is based on IC50 values for hAldh1a3 and hAldh1a2. The designations for the hAldh1a3:hAldh1a2 ratio are as follows: A<0.09; B=0.1-0.2; C>0.21 and for hAld1a3 and mAldh1a3 are as follows: D<100 nM; E=100-200 nM; and F>200 nM.

Control compounds A-C are disclosed in US 2024/0132469 which is incorporated by reference herein.

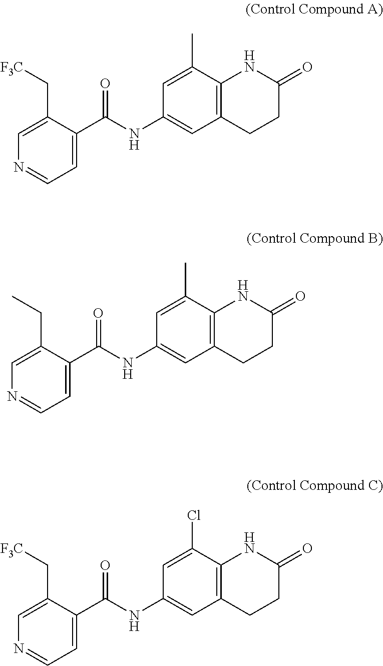 [SMILES: Cc1cc(NC(=O)c2ccncc2CC(F)(F)F)cc2c1NC(=O)CC2 ; CCc1cnccc1C(=O)Nc1cc(C)c2c(c1)CCC(=O)N2 ; O=C1CCc2cc(NC(=O)c3ccncc3CC(F)(F)F)cc(Cl)c2N1]

TABLE 2
Selectivity of hALDH1a3 v. hALDH1a2, mALDH1a3,
and hALDH1a3 of Selected Compounds

| | | hAldh1a3 | mAldh1a3 |
|---|---|---|---|
| No. | hAldh1a3:hAldh1a2 | IC50 | IC50 |
| Control Compound A | C | D | D |
| Control Compound B | C | D | D |
| Control Compound C | C | D | D |
| 1 | A | D | F |
| 2 | A | D | E |
| 3 | B | D | F |
| 4 | B | E | F |
| 5 | B | D | D |
| 6 | B | F | F |
| 7 | B | F | F |
| 8 | A | F | F |
| 9 | A | D | E |
| 10 | B | E | E |
| 11 | B | E | D |
| 12 | A | D | D |
| 13 | A | D | D |
| 14 | A | F | D |
| 15 | B | D | E |
| 16 | A | E | F |
| 17 | B | E | D |
| 18 | B | D | D |
| 19 | B | E | D |
| 20 | C | D | D |
| 21 | C | E | D |
| 22 | B | F | F |
| 23 | A | F | F |
| 24 | B | F | E |
| 25 | C | E | D |
| 26 | A | E | E |

### Example 22: CYP3A4 Inhibition Assay

To assess the inhibition of CYP3A4 using human liver microsomes (HLM), a protocol was performed utilizing midazolam as the CYP3A4 substrate and ketoconazole as a positive control inhibitor, with both stock solutions prepared (midazolam in MeOH and ketoconazole in DMSO) and the ketoconazole working solution generated via dilution. The enzyme source was HLM, and incubations were carried out in 0.1 M phosphate buffer (pH 7.4) at 37° C. A 10× cocktail of CYP substrates, inclusive of midazolam, and a 1.27× working solution of HLM were prepared. Substrate solutions were added to appropriate wells, with phosphate buffer used for blanks, and test compound working solutions and the ketoconazole positive control were added to their respective wells, with solvent and phosphate buffer used for no-inhibitor and blank wells, respectively. Subsequently, the HLM working solution was added to all wells of the incubation plate, which was then pre-warmed at 37° C. Following preparation of the NADPH cofactor solution, the plate was pre-incubated for 10 minutes at 37° C. with the test compounds or ketoconazole and HLM, after which the reaction was initiated by adding the NADPH cofactor. After a 10-minute incubation, the reaction was quenched with a cold stop solution containing tolbutamide and labetalol in acetonitrile. The reaction mixture was then centrifuged, and the resulting supernatant was diluted with water. The diluted samples were analyzed by LC/MS/MS to quantify the formed midazolam metabolite, thus enabling the evaluation of CYP3A4 inhibition. Finally, the inhibition observed with the test compounds was compared to that of the positive control and historical data for analysis.

The Cyp3A4% inhibition for selected test compounds of the disclosure is shown in Table 3 below. The Cyp3A4% inhibition values are designated as follows: A≥25; B<25 at 10 μM.

TABLE 3
CYP3A4 % inhibition of selected compounds

| | Cyp3A4 |
|---|---|
| Compound | % inhibition |
| 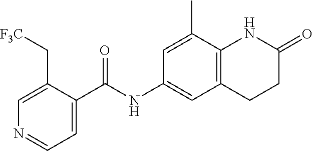 [SMILES: Cc1cc(NC(=O)c2ccncc2CC(F)(F)F)cc2c1NC(=O)CC2] | A |
| Control Compound A | |
| 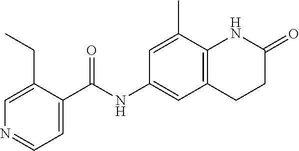 [SMILES: CCc1cnccc1C(=O)Nc1cc(C)c2c(c1)CCC(=O)N2] | A |
| Control Compound B | |
| 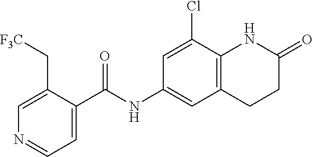 [SMILES: O=C1CCc2cc(NC(=O)c3ccncc3CC(F)(F)F)cc(Cl)c2N1] | A |
| Control Compound C | |
| 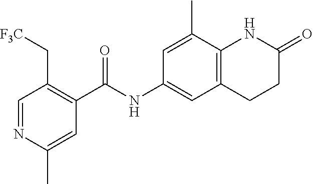 [SMILES: Cc1cc(C(=O)Nc2cc(C)c3c(c2)CCC(=O)N3)c(CC(F)(F)F)cn1] | B |
| Compound 1 | |
| 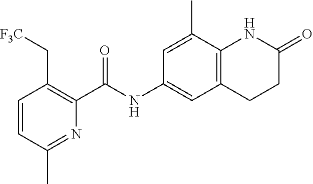 [SMILES: Cc1ccc(CC(F)(F)F)c(C(=O)Nc2cc(C)c3c(c2)CCC(=O)N3)n1] | B |
| Compound 2 | |
| 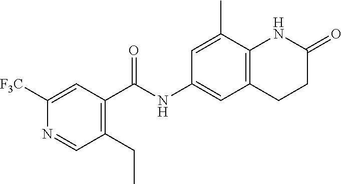 [SMILES: CCc1cnc(C(F)(F)F)cc1C(=O)Nc1cc(C)c2c(c1)CCC(=O)N2] | B |
| Compound 3 | |
| 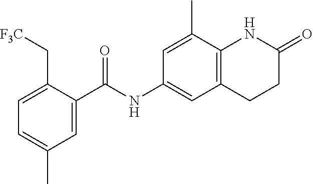 [SMILES: Cc1ccc(CC(F)(F)F)c(C(=O)Nc2cc(C)c3c(c2)CCC(=O)N3)c1] | A |
| Compound 5 | |
| 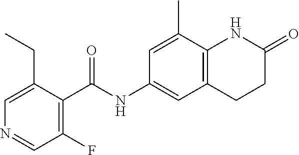 [SMILES: CCc1cncc(F)c1C(=O)Nc1cc(C)c2c(c1)CCC(=O)N2] | A |
| Compound 6 | |
| 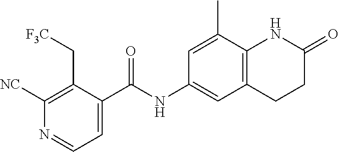 [SMILES: Cc1cc(NC(=O)c2ccnc(C#N)c2CC(F)(F)F)cc2c1NC(=O)CC2] | B |
| Compound 9 | |
| 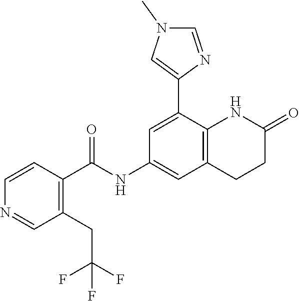 [SMILES: Cn1cnc(-c2cc(NC(=O)c3ccncc3CC(F)(F)F)cc3c2NC(=O)CC3)c1] | A |
| Compound 11 | |
| 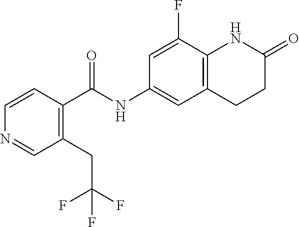 [SMILES: O=C1CCc2cc(NC(=O)c3ccncc3CC(F)(F)F)cc(F)c2N1] | B |
| Compound 15 | |
| 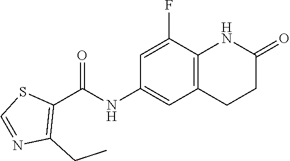 [SMILES: CCc1ncsc1C(=O)Nc1cc(F)c2c(c1)CCC(=O)N2] | B |
| Compound 17 | |
| 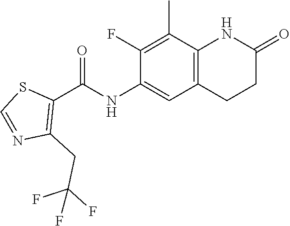 [SMILES: Cc1c(F)c(NC(=O)c2scnc2CC(F)(F)F)cc2c1NC(=O)CC2] | B |
| Compound 18 | |
| 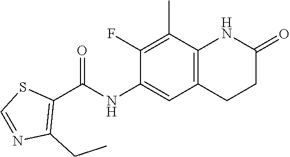 [SMILES: CCc1ncsc1C(=O)Nc1cc2c(c(C)c1F)NC(=O)CC2] | B |
| Compound 19 | |

### Example 23: Evaluation of Pharmacokinetic Profile

The pharmacokinetic profile of selected compounds was evaluated.

Study Details

| | | |
|---|---|---|
| Species | Dog (n = 3/group) | |
| Study Group | intravenously (iv) | orally (p.o.) |
| Dose of Test | 1 mg/kg (iv) | 10 mg/kg (p.o.) |
| Compound | | |

Bioanalysis Details

| | |
|---|---|
| Analytical | LC-MS/MS |
| technique | |
| Matrix | Plasma (EDTA-K2) |

PK Calculation Settings

| | |
|---|---|
| Program | Phoenix WinNonlin 8.3.5 |
| Model | IV-Noncompartmental model 201 (IV bolus input) |
| | PO-Noncompartmental model 200 (extravascular input) |
| Calculation | Linear/log trapezoidal |
| method | |

* “BQL” was excluded in the PK parameters and mean concentration calculation.

### Results:

The mean oral T1/2 (h) for selected test compounds of the disclosure is shown in Table 4 below.

TABLE 4
T1/2 (h) for selected test compounds.

| Compound | T ¹/₂ (h) |
|---|---|
| 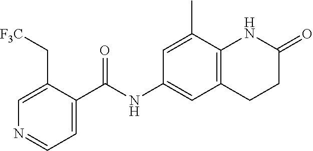 [SMILES: Cc1cc(NC(=O)c2ccncc2CC(F)(F)F)cc2c1NC(=O)CC2] | 16.4 |
| Control Compound A | |
| 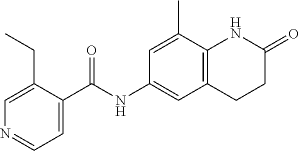 [SMILES: CCc1cnccc1C(=O)Nc1cc(C)c2c(c1)CCC(=O)N2] | 2.69 |
| Control Compound B | |
| 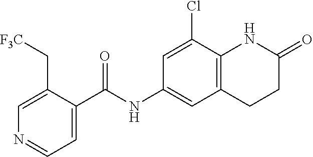 [SMILES: O=C1CCc2cc(NC(=O)c3ccncc3CC(F)(F)F)cc(Cl)c2N1] | 2.64 |
| Control Compound C | |
| 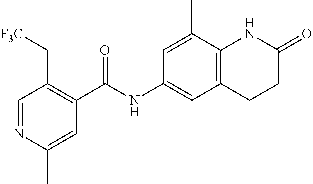 [SMILES: Cc1cc(C(=O)Nc2cc(C)c3c(c2)CCC(=O)N3)c(CC(F)(F)F)cn1] | 38.9 |
| Compound 1 | |

As shown in Table 4, Compound 1 of the present disclosure showed better PK characteristics for oral administration, such as significantly improved half-life (t1/2) compared to structurally similar control compounds.

### Example 24: Evaluation of Compound 1 in a Pre-Type 2 Diabetes Model

The efficacy of Compound 1 in preventing type 2 diabetes onset in pre-diabetic ZDF rats was evaluated.

### Protocol

Homozygous Zucker Diabetic Fatty (ZDF) male rats were screened and randomized into two groups (n=10/group) based on HOMA-IR, HbA1c and body weight and administered 30 mg/kg of Compound 1 or vehicle p.o. once daily with for 7 weeks.

TABLE 5
Study treatments

| Group | Nbr/ | | | | Treatment |
|---|---|---|---|---|---|
| No | group | Treatment | Route | Dose | duration |
| 1 | 10 | vehicle | Oral, QD | / | 7 weeks (52 days) |
| 2 | 10 | Compound 1 | Oral, QD | 30 mg/kg | 7 weeks (52 days) |

Fasting blood glucose, plasma insulin and HbA1c levels were measured every two weeks and HOMA-IR was calculated for each measurement. Water and food intakes per cage and body weight per animal were measured once a week. Fasting serum chemistry was measured at initiation, midpoint and endpoint of the study. At day 48 of treatment, an intraperitoneal glucose tolerance test (ipGTT) was performed with plasma insulin and c peptide levels measured at time 0 and +30 minutes after the intraperitoneal glucose injection. At the end of treatment period, animals were fasted for 8 hours after dosing, then blood was collected to measure endpoint parameters, including clinical chemistry. Kidneys, liver, mWAT and iWAT and pancreas were collected for formalin-fixation and/or freezing.

### Results

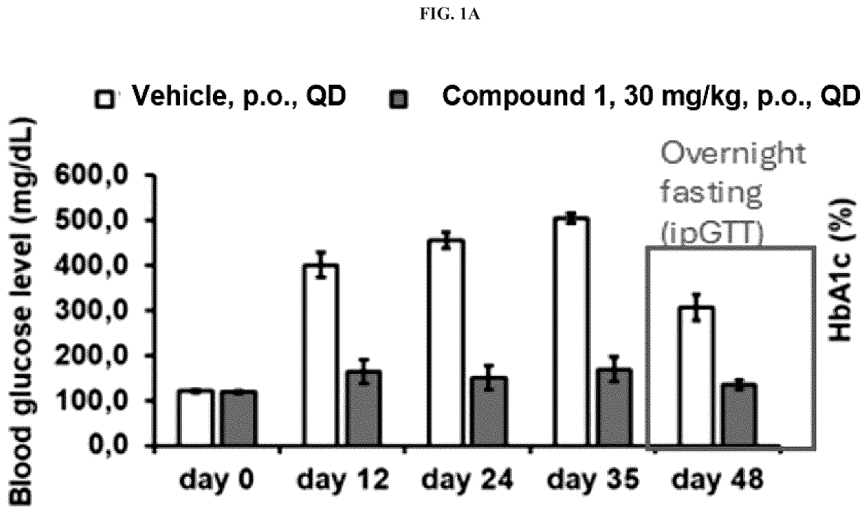

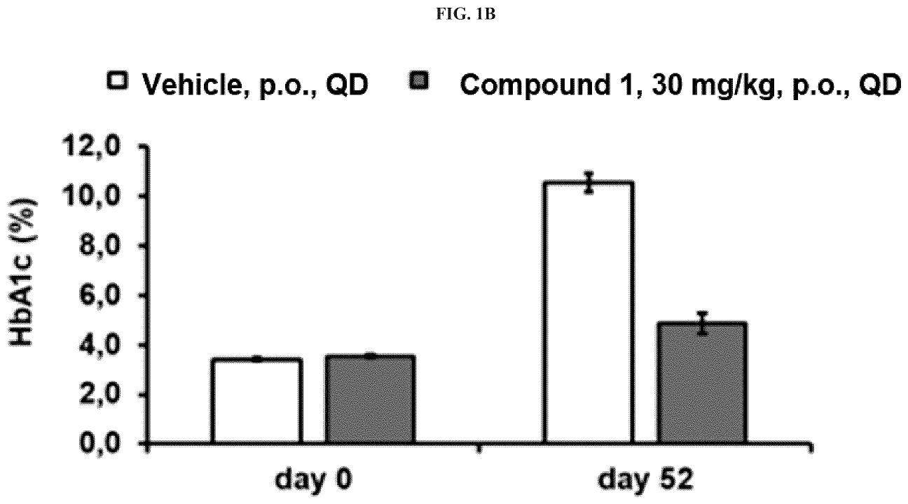

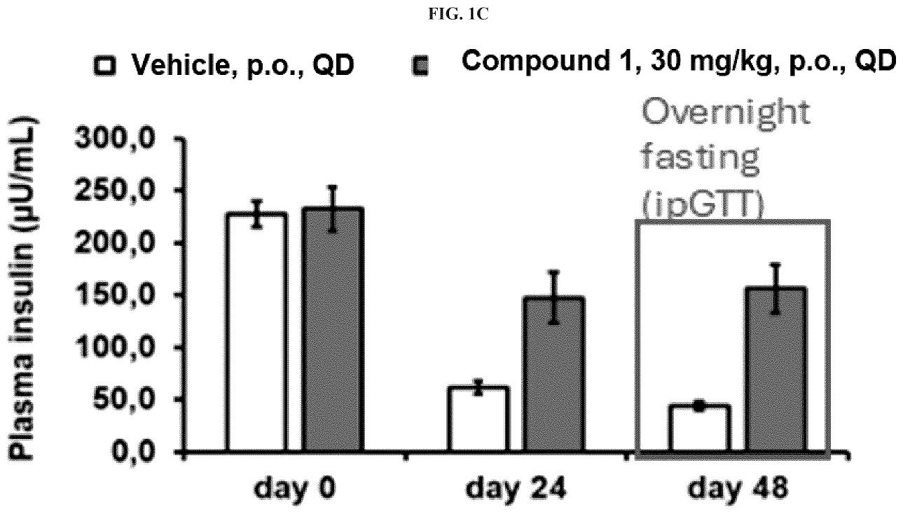

Compound 1 showed a strong anti-diabetic effect, completely preventing diabetic onset by fasting blood glucose (FIG. 1A), HbA1c (FIG. 1B) and insulin levels (FIG. 1C). As shown in FIG. 1A Compound 1 treatment maintained healthy fasting blood glucose levels compared to significant increase in fasting blood glucose in vehicle treated rats. Compound 1 treatment also significantly improved glucose tolerance compared to vehicle during an intraperitoneal glucose tolerance test and resulted in increased beta cell function as measured by plasma insulin levels 30 minutes after i.p. glucose injection. This positive effect was associated with a significant reduction in % HbA1c at the end of treatment period (FIG. 1B) and fasting insulin was also significantly improved by Compound 1 treatment (FIG. 1C). These results show that Compound 1 treatment was able to successfully prevent the onset of Type 2 diabetes.

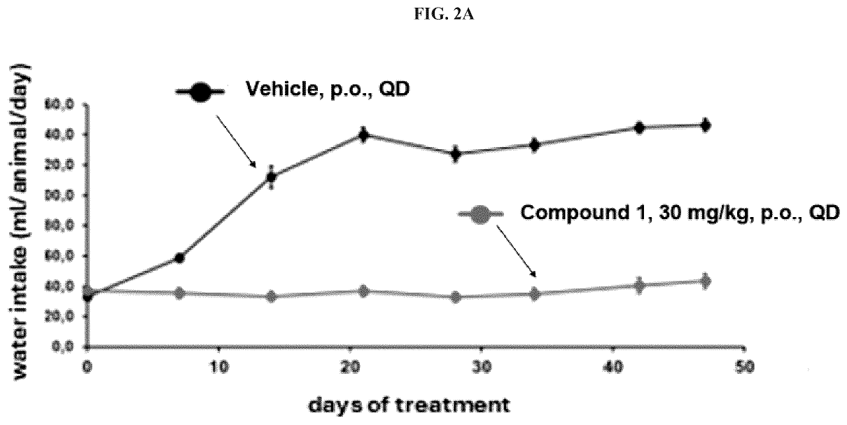

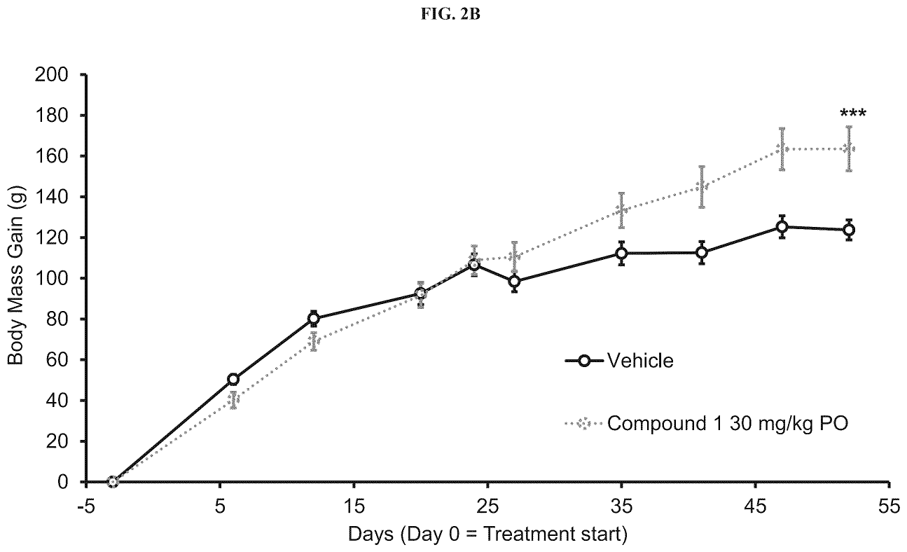

Compound 1 treatment over 7 weeks also led to improved kidney function and reduced glycosuria as measured by reduced water intake (FIG. 2A) and an increase in healthy body mass gain (FIG. 2B), reflecting improved insulin sensitization.

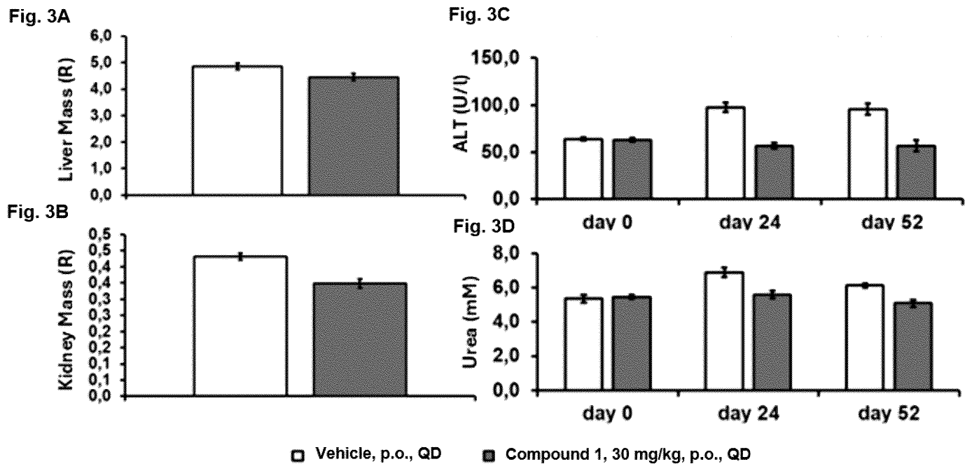

Reduced liver (FIG. 3A) and kidney weights (FIG. 3B) were also observed, suggesting reduced steatosis and fibrotic content. Reduced blood urea (FIG. 3C), ALT (FIG. 3D) and water intake (FIG. 2A) strongly suggests improved liver and kidney health in Compound 1 treated animals. Animal technicians also observed that test and control animals were visually distinguishable as Compound 1-treated animals were healthy with no outward signs of diabetic onset.

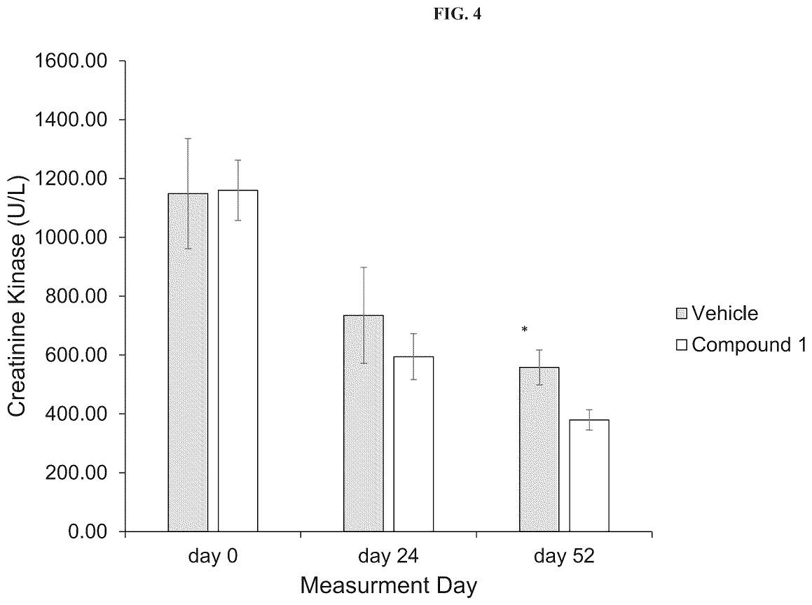

Creatine kinase was reduced overtime and significantly reduced under Compound 1 treatment, when compared to vehicle treatment at the last day of treatment (−32%, p<0.05, FIG. 4) suggesting a decrease of muscle mass damage.

### Example 25: Evaluation of Compound 1 in a Model of Severe Type 2 Diabetes

The efficacy of Compound 1 in treating type 2 diabetes in severely diabetic db/db mice was evaluated.

### Protocol

16 and 17-week old, male, severely diabetic db/db mice were used for the study. Animals were dosed daily for 4 weeks with either vehicle control or 50 mg/kg of Compound 1 administered orally.

### Results

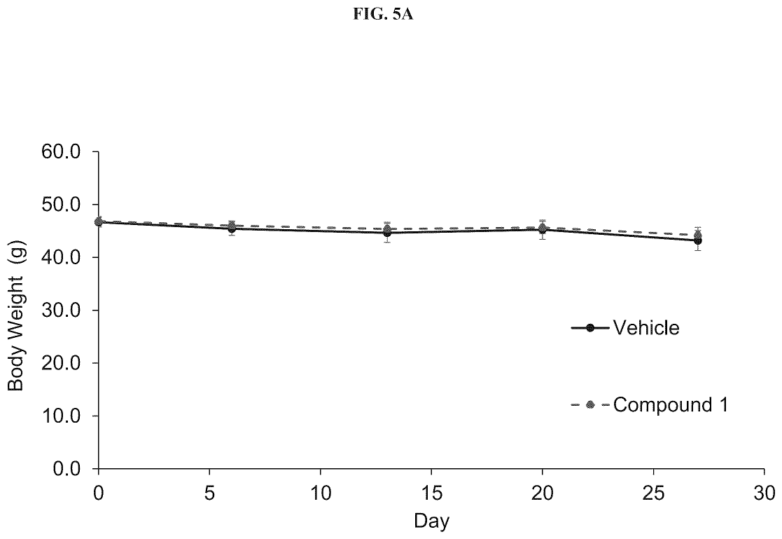

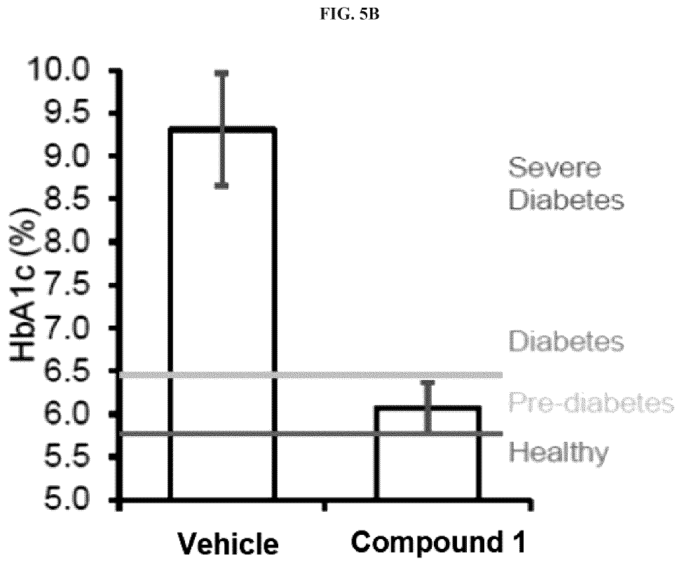

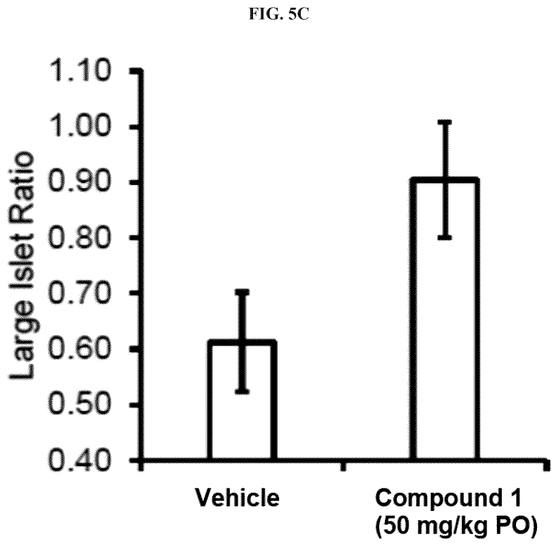

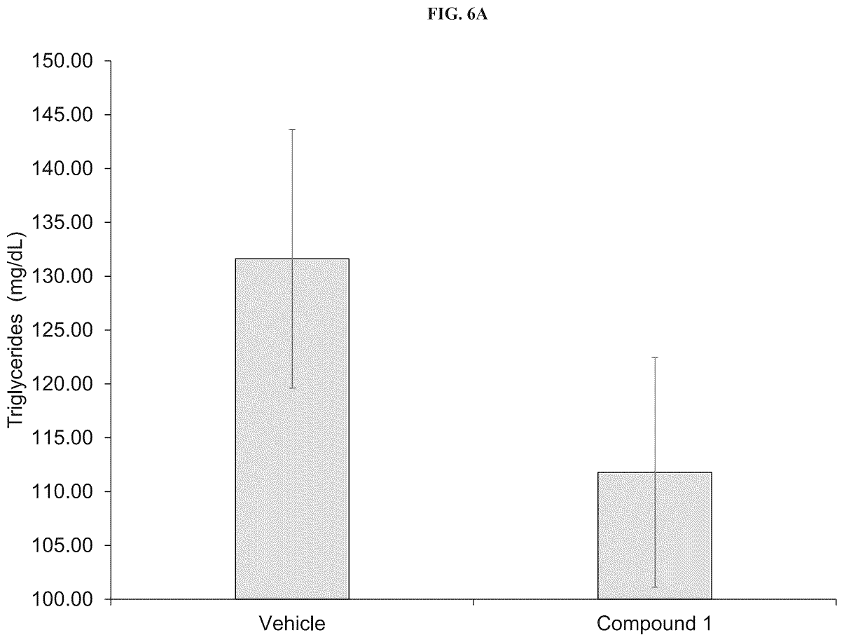

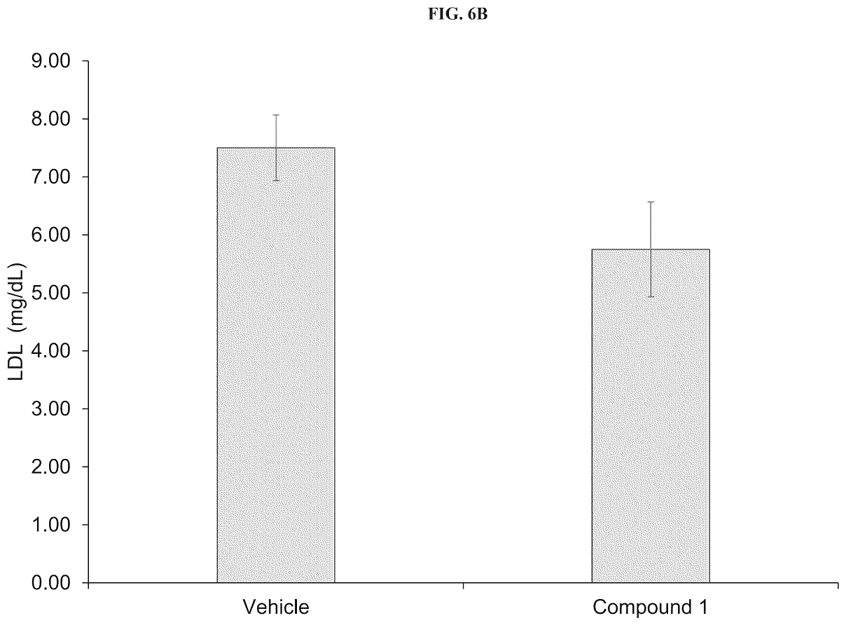

Treatment of severely diabetic db/db mice with Compound 1 over 28 days showed an almost complete reversal of type 2 diabetes as indicated by a decrease in HbA1c levels (FIG. 5B) and a recovery in pancreatic large islet ratio (FIG. 5C) without affecting food intake or body weight (FIG. 5A). Clinical chemistry demonstrated a striking 30% reduction in triglycerides (FIG. 6A) and 17% reduction in LDLs (FIG. 6B) in the Compound 1 treated group.

These results show that Compound 1 treatment was successful in reversing diabetes in severely diabetic db/db mice.

### Example 26: Compound 1 v. Semaglutide in Spontaneously Diabetic Torii (STD Fatty Rat Model of Diabetic Heart Failure

The following is a head-to-head study of Compound 1 and Semaglutide in a SDT fatty rat model of diabetic heart failure.

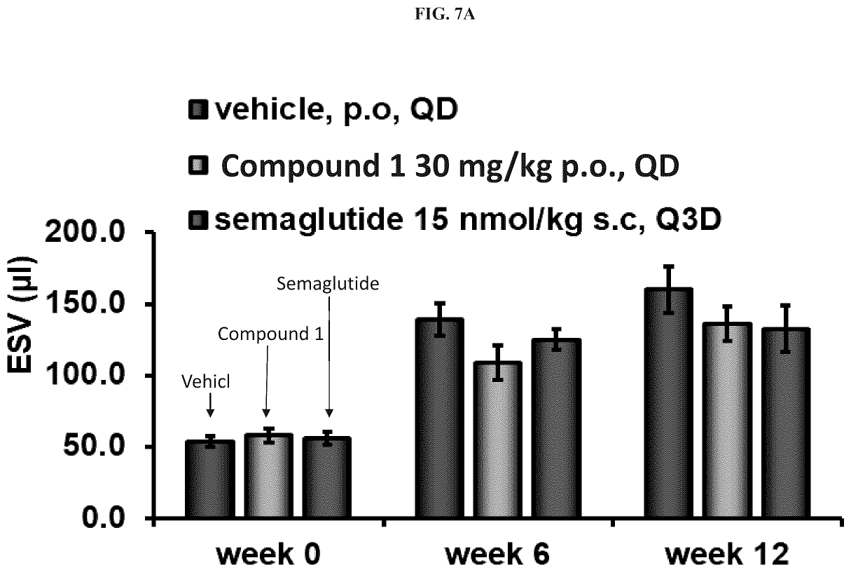

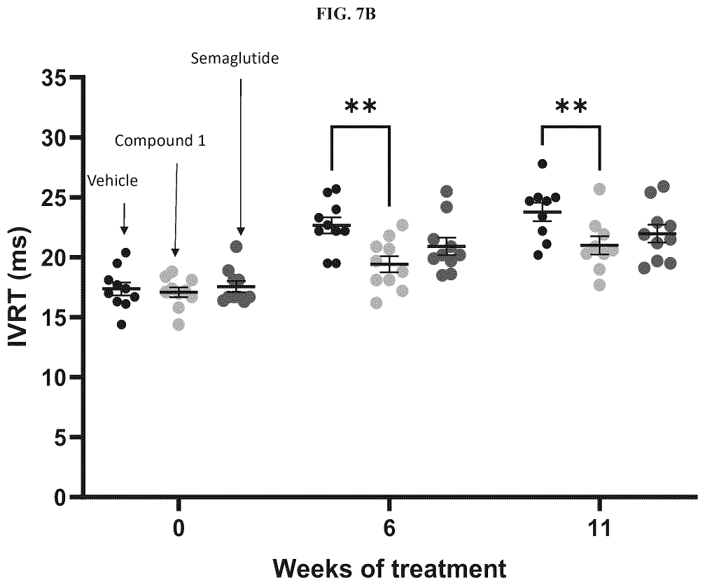

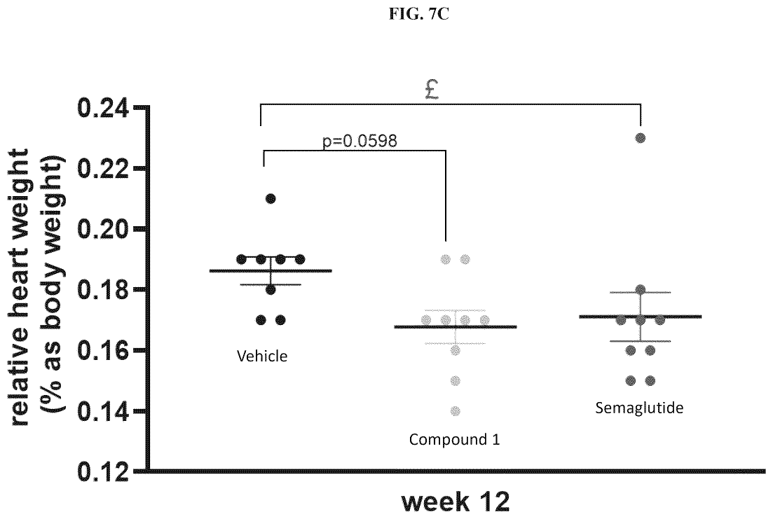

Diabetic 7-week old SDT fatty rats were treated with vehicle, Compound 1 (30 mg/kg p.o. QD) or Semaglutide (15 nmol/kg s.c. Q3D) for 12 weeks with echocardiography used to measure cardiac dynamics (FIG. 7A). End-Systolic Volume measures degree of cardiac remodeling.

Isovolumetric relaxation time was measured as the key measure of heart failure with preserved ejection fraction (HFpEF) across each group (FIG. 7B).

At endpoint, animals were sacrificed and heart mass was collected for each group. n=10 rats/group. Error bars represent SEM (FIG. 7C).

### Results

A head-to-head study of Compound 1 vs Semaglutide in this diabetic model of HFpEF demonstrates both therapies are effective in slowing the cardiac remodeling that leads to heart failure.

Compound 1 is more efficient in slowing isovolumic relaxation time (IVRT) increases.

1. A compound of Formula (III-D): 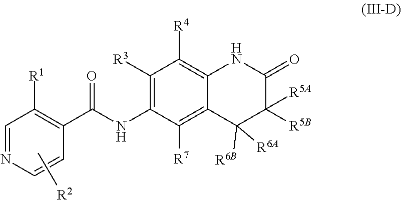
or a pharmaceutically acceptable salt, stereoisomer, tautomer, or deuterated form thereof wherein:
R¹ is —C₁₋₆ haloalkyl, halo, or —NR⁸R⁹;
R² is independently halo, —CN, —C₁₋₆ alkyl, —C₁₋₆ alkylene-carbocyclyl, —C₁₋₆ alkylene-heterocyclyl, or —C₁₋₆ haloalkyl;
R³ is —H, halo, —C₁₋₆ alkyl, or —C₁₋₆ haloalkyl;
R⁴ is halo, —CN, —OH, —O—C₁₋₆alkyl, —C₁₋₆alkyl, —C₁₋₆ alkyl-CN, —C₁₋₆ haloalkyl, —C(O)NR⁸R⁹, —C(O)OR⁸, —NR⁸R⁹, —NR⁸C(O)R⁹, heterocyclyl, heteroaryl, or carbocyclyl; or R³ and R⁴ are taken together to form heteroaryl, carbocyclyl, or heterocyclyl, each of which may be substituted with one or more halo, —C₁₋₆ haloalkyl, —C₁₋₆alkyl, —CN, —OH, —NH₂, —NH(alkyl), or —N(alkyl)₂;
R^{5A}, R^{5B}, R^{6A}, and R^{6B} are each independently —H, halo, or —C₁₋₆ alkyl; or
one of R^{5A} and R^{5B} is joined with one of R^{6A} and R^{6B} to form an optionally substituted C₃₋₈ carbocyclic ring, or an optionally substituted 3-8 membered heterocyclic ring; and
R⁷ is —H, —C₁₋₆alkyl or halo; and
R⁸ and R⁹ are each independently —H, —C₁₋₆ alkyl, aryl or heteroaryl.

2. The compound of claim 1, wherein R¹ is —C₁₋₆ haloalkyl, halo, or —NR⁸R⁹.

3. The compound of claim 1, wherein R¹ is —C₁₋₆ haloalkyl.

4. The compound of claim 3, wherein R¹ is —CH₂—CF₃.

5. The compound of claim 1, wherein R² is —C₁₋₆ alkyl, -halo, or —CN.

6. The compound of claim 1, wherein R¹ is —C₁₋₆ haloalkyl and R² is —C₁₋₆ alkyl or —CN.

7. The compound of claim 1, wherein R³ is —H.

8. The compound of claim 1, wherein R⁴ is —C₁₋₆ alkyl, —NR⁸R⁹, -heteroaryl, -or halo.

9. The compound of claim 1, wherein R⁴ is —CH₃.

10. The compound of claim 1, wherein R⁴ is -halo.

11. The compound of claim 1, wherein R⁴ is —NH₂.

12. The compound of claim 1, wherein R⁴ is —C₁₋₆ alkyl, —NR⁸R⁹, -heteroaryl, -or halo and R³ is —H.

13. The compound of claim 1, wherein R^{5A}, R^{5B}, R^{6A}, and R^{6B} are each —H.

14. The compound of claim 1, wherein R⁷ is —H.

15. The compound of claim 1, wherein the compound is selected from the group consisting of: 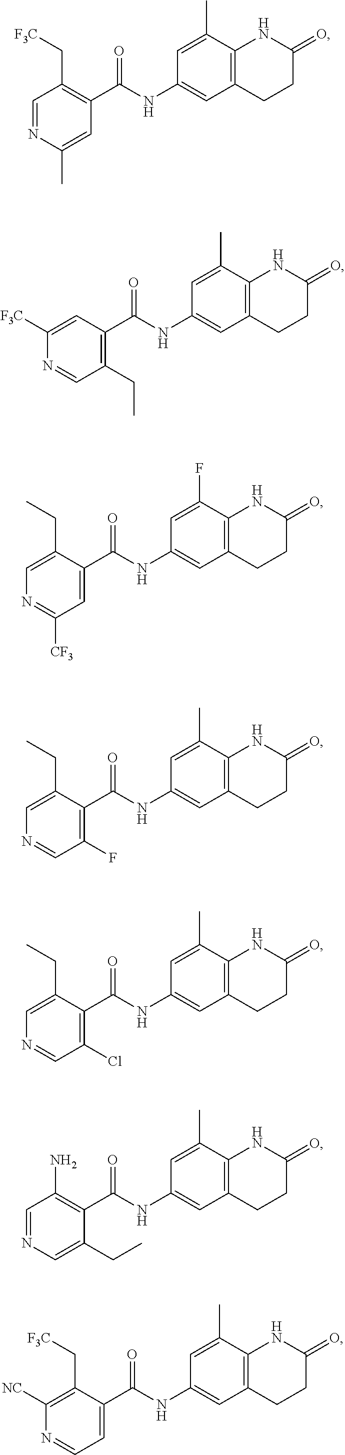 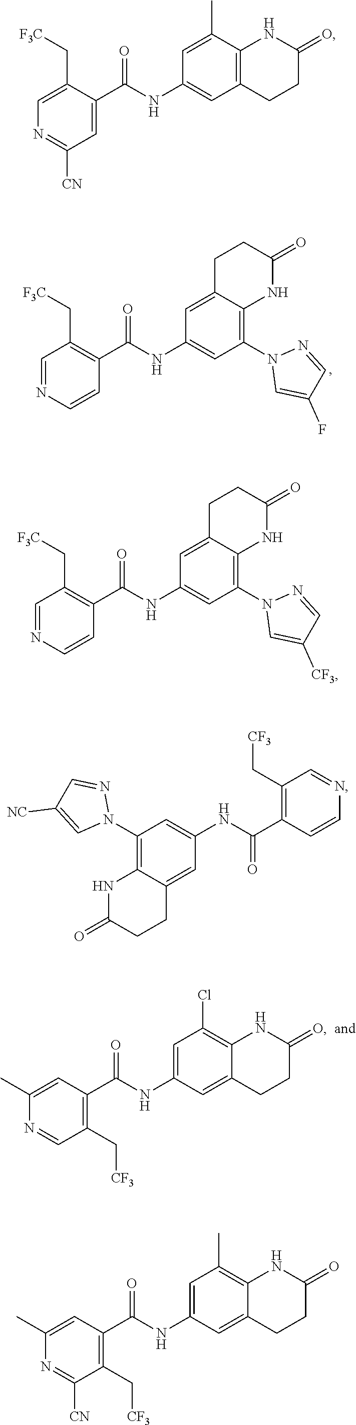
or a pharmaceutically acceptable salt, stereoisomer, tautomer, or deuterated form thereof.

16. A method of treating disease or disorder in a subject in need thereof, the method comprising administering a compound of claim 1 to the subject, wherein the disease or disorder is cardiometabolic syndrome, a diabetic kidney disease, a lipid disorder, muscle inflammation, sarcopenia or bone density loss, non-alcoholic fatty liver disease (NAFLD), non-alcoholic steatohepatitis (NASH), metabolic dysfunction-associated steatohepatitis (MASH), hypercholesterolemia, hypertriglyceridemia, familial cholesterolemia, familial triglyceridemia, or a cardiovascular disorder.

17. The method of claim 16, wherein the lipid disorder is hyperlipidemia or dyslipidemia.

18. The method of claim 16, wherein the cardiovascular disorder is pulmonary arterial hypertension, chronic heart failure, neointimal hyperplasia, Atherosclerotic Cardiovascular Disease (ASCVD), heart failure with preserved ejection fraction, ischemic heart disease or coronary artery disease.
